(19) United States
(12) Patent Application Publication
(10) Pub. No.: US 2010/0144645 A1
(43) Pub. Date: Jun. 10, 2010

(54) COMPOSITIONS AND METHODS FOR ENHANCING ANALGESIC POTENCY OF COVALENTLY BOUND-COMPOUNDS, ATTENUATING ITS ADVERSE SIDE EFFECTS, AND PREVENTING THEIR ABUSE

(75) Inventors: Randal J. Kirk, Radford, VA (US); Suma Krishnan, Belvedere, CA (US); James Scott Moncrief, Christiansburg, VA (US)

Correspondence Address: DARBY & DARBY P.C., P.O. BOX 770, Church Street Station, New York, NY, 10008-0770, US

(73) Assignee: SHIRE LLC, Florence, KY (US)

(21) Appl. No.: 12/296,715

(22) PCT Filed: Apr. 16, 2007
(86) PCT No.: PCT/US2007/009220

§ 371 Date: Feb. 16, 2010

Related U.S. Application Data
(60) Provisional application No. 60/791,892, filed on Apr. 14, 2006.
(60) Provisional application No. 60/796,352, filed on May 1, 2006.
(60) Provisional application No. 60/849,775, filed on Oct. 6, 2006.
(60) Provisional application No. 60/849,776, filed on Oct. 6, 2006.
(60) Provisional application No. 60/849,774, filed on Oct. 6, 2006.

(51) Int. Cl. A61K 38/08; A61K 31/485; A61K 38/05; A61K 38/06; A61K 38/07; A61P 25/36

(52) U.S. Cl. 514/16; 514/282; 514/19; 514/18; 514/17

(57) ABSTRACT

The invention generally relates to compositions and methods with covalently bound compounds, such as controlled substances covalently attached to a chemical moiety, and opioid antagonists or covalently bound opioid antagonists to enhance analgesic potency and/or attenuate one or more adverse effects of covalently bound compounds, including adverse side effect(s) in humans such as nausea, vomiting, dizziness, headache, sedation (somnolence), physical dependence or pruritis. This invention relates to compositions and methods for selectively enhancing the analgesic potency of a covalently bound compound and simultaneously attenuating anti-analgesia, hyperalgesia, hyperexcitability, physical dependence and/or tolerance effects associated with the administration of a covalently bound compound. The methods of the invention comprise administering to a subject an analgesic or sub-analgesic amount of a covalently bound compound and an amount of excitatory opioid receptor antagonist such as naltrexone or nalmefene effective to enhance the analgesic potency of a covalently bound compound and attenuate the anti-analgesia, hyperalgesia, hyperexcitability, physical dependence and/or tolerance effects of covalently bound compound. The invention also relates to the addition of covalently-bound opioid antagonists to the compositions containing covalently bound compounds such that if the compositions are subjected to manipulation by illicit chemists, the opioid antagonist is released effectively reducing or eliminating the euphoric effect of the covalently bound compounds.

### CROSS-REFERENCE RELATED APPLICATIONS

This application claims benefit under 35 U.S.C. §119(e) to U.S. Provisional Application No. 60/796,352 filed on May 1, 2006, claims benefit under 35 U.S.C. §119(e) to U.S. Provisional Application 60/849,776 filed on Oct. 6, 2006, claims benefit under 35 U.S.C. §119(e) to U.S. Provisional Application 60/849,775 filed Oct. 6, 2006, claims benefit under 35 U.S.C. §119(e) to U.S. Provisional Application 60/849,774 filed Oct. 6, 2006, and claims benefit under 35 U.S.C. §119(e) to U.S. Provisional Application 60/791,892 filed Apr. 14, 2006 each of which are hereby incorporated by reference in their entirety.

### BACKGROUND OF THE INVENTION

Morphine or other bimodally-acting opioid agonists are administered to relieve severe pain due to the fact that they have analgesic effects mediated by their activation of inhibitory opioid receptors on nociceptive neurons (see North, Trends Neurosci., Vol. 9, pp. 114-117 (1986) and Crain and Shen, Trends Pharmacol. Sci., Vol. 11, pp. 77-81 (1990)). However, morphine and other bimodally-acting opioid agonists also activate opioid excitatory receptors on nociceptive neurons, which attenuate the analgesic potency of the opioids and result in the development of physical dependence and increased tolerance (see Shen and Crain, Brain Res., Vol. 597, pp. 74-83 (1992)), as well as hyperexcitability, hyperalgesia and other undesirable (excitatory) side effects. As a result, a long-standing need has existed to develop a method of both enhancing the analgesic (inhibitory) effects of bimodally-acting opioid agonists and blocking or preventing undesirable (excitatory) side effects caused by such opioid agonists.

U.S. Pat. Nos. 5,472,943; 5,512,578; 5,580,876; 5,767,125; 6,096,756; 6,362,194; as well as U.S. Patent Application Publications 2002/0094947 A1 and 2001/0006967 A1 all teach an analgesic or sub-analgesic amount of an opioid agonist and an amount of an excitatory opioid receptor antagonist effective to enhance the analgesic potency and attenuate the anti-analgesia, hyperalgesia, hyperexcitability, physical dependence and/or tolerance effects. However, none of these references teach or suggest the presence of covalently bound controlled substances in conjuntion with an excitatory opioid receptor antagonist that may be covalently bound.

In addition, there is considerable information readily available to individuals which teaches how to derive purified forms of controlled substances from prescription products resulting in the abuse of these controlled substances. These techniques are both simple and well described on multiple websites. Most of these procedures utilize cold water, although, hot water, changes in pH and other solvents are described. Examples of these procedures are described below.

The description of these procedures was found on the web in February of 2003 at http://codeine.50 g.com/info/extraction.html#ex.coldw and is paraphrased below. Cold water extraction is used to extract an opiate/opioid substance from combination tablets. This method subverts the fact that opiates are generally very soluble in cold water, while paracetamol, aspirin, and ibuprofen are only very slightly soluble. These techniques are sophisticated enough to recognize that pseudoephedrine and caffeine are water soluble and will remain in the solution and that dispersible tablets make it difficult to extract secondary substances. The description of the equipment required makes it clear that these procedures make abuse readily available. The equipment includes a minimum two glasses or cups, paper filters (unbleached coffee filters will do) and a measure glass. Portions of the procedures are provided below:
1. Crush the tablets and dissolve in cold (20° C.) water.
2. Cool the solution down to approximately 5° C. stirring occasionally.
3. Leave the solution in a cool place for about 20 minutes.
4. Wet the filter(s) with very cold water to prevent it from absorbing the solution and put it in the glass. Stick an elastic/rubber band around the container to keep the filter in place.
5. Pour the solution through the filter to filter out the secondary substance from codeine.
6. Discard used filters with secondary substance solids left.

However, when these procedures were viewed as not providing sufficient yields improved method were designed for extracting codeine which simply require the addition of chloroform or like solvent such as methylene chloride. This technique utilizes methods which alter the pH aspects of the solution to improve extraction and even provides instruction on how to re-salt the product. Portions of the procedure are described below.
1. Place uncrushed T3's or other APAP/codeine product in a small glass or beaker and cover with enough distilled water so that the pills will break down into a thin paste.
2. Add dry sodium carbonate to reduce the codeine phosphate to codeine base. The pH of the mixture should be about 11 or greater.
3. Pour the mixture into the pyrex pan and rinse the beaker with a few ml of distilled water and add the rinse water to the mix in a pan.
4. Wrap the dried material in a coffee filter and grind the stuff
5. Pour the dry crushed mixture into a glass bottle with a screw-on top and pour in enough chloroform to completely cover.
6. Shake and filter.

While there has been considerable effort to provide controlled substances which are resistance to abuse current products fail to achieve the stability required to prevent abuse. The present invention provides methods and compositions which retain their stability even when subjected to current abuse methods, or in the alternative, release an opioid antagonist effectively blocking the euphoric effect of the controlled substances when the controlled substances are subjected to the above described procedures and therefore provide a much needed but less addictive and/or less likely to be abused product.

In addition to highly efficacious analgesic activity, opioids such as morphine commonly produce unwanted and sometimes disturbing side effects which are also mediated by its agonist activity at the mu-opioid receptor. These include the depression of respiratory rate leading to death in cases of overdose and decreased bowel motility. Years of research and development of morphine analogs, with a variety of affinities and intrinsic activities at mu and other subtypes of opioid receptors, have failed to address these problems. In addition, chronic opioid administration used to treat chronically painful conditions such as cancer can be complicated by the development of drug dependence, addiction, and tolerance. The latter effect results in the need for increasing doses of the drug which tends to coincide with increased frequency of many adverse events.

US 2004/0024005 A1, which is herein incorporated by reference in its entirety, discloses a composition, where the analgesic is present in a sub-analgesic amount in order to reduce, prevent or delay the development of tolerance to and/or physical dependence on the particular agonist that target G-protein coupled receptors (GPCRs), which include mu opioid receptors. The composition includes a combination of an agonist that targets a GPCR, where the agonist does not promote endocytosis and resensitization of the targeted GPCR, and an agonist that promotes the endocytosis of the GPCR and is present in the composition in an amount sufficient to promote endocystosis and resensitization of the targetted GPCR. The composition includes an opioid agonist such as morphine, and a mu opioid receptor agonist that includes methadone, fentanyl, sulfentanil, remi-fentanyl, etonitazene, and etorphine.

Among the various adverse events reported with the use of controlled substances (e.g. opioids), constipation stands out as one of the most persistent and debilitating. It would be desirable to preserve the pain-relieving actions of controlled substances (e.g. opioids) while blocking the constipating actions of controlled substances (e.g. opioids). For instance, reduced constipation may be achieved through the addition of low dose racemic methadone to controlled substances (e.g. opioids) such as morphine. It has been found that morphine, unlike other agonists such as methadone and fentanyl, binds to the receptor and initiates cellular responses leading to analgesia but it does not induce endocytosis like the other agonists.

### BRIEF DESCRIPTION OF DRAWINGS

FIG. 1 depicts the numbering scheme for hydrocodone.

FIG. 2 depicts hydrocodone conjugated at the 6 position.

FIG. 3 depicts the numbering scheme for hydromorphone.

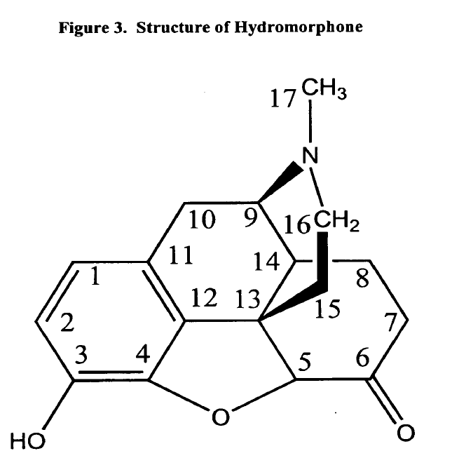

FIG. 4 depicts hydromorphone conjugated at the 3 position.

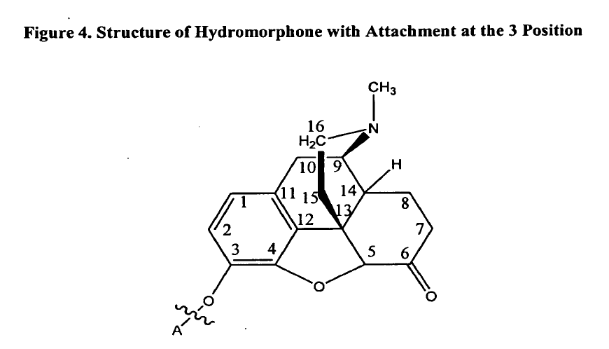

FIG. 5 depicts hydromorphone conjugated at the 3 and 6 positions.

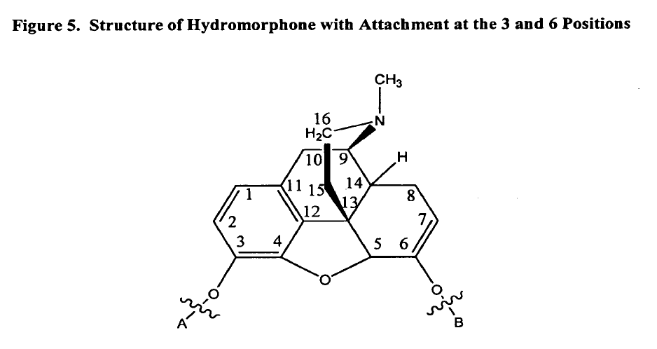

FIG. 6 depicts hydromorphone conjugated at the 6 position.

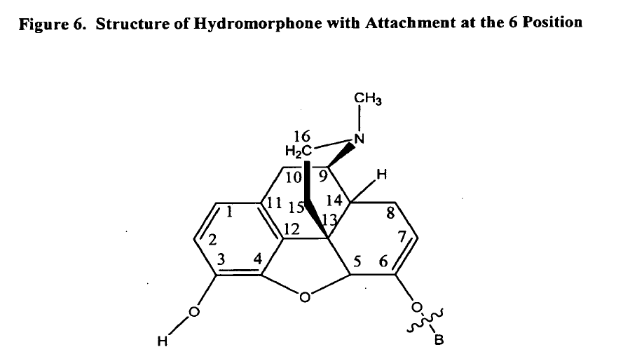

FIG. 7 depicts the numbering scheme for oxycodone.

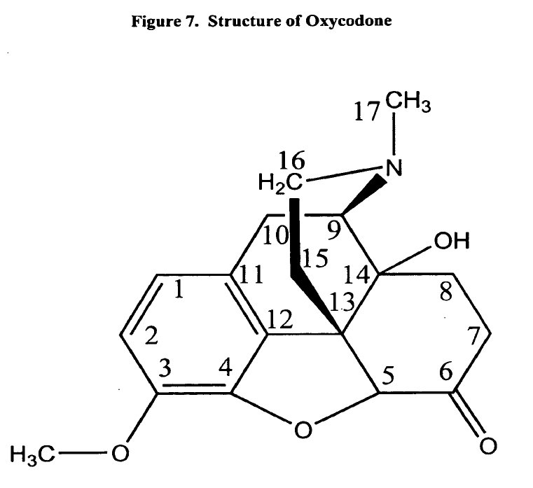

FIG. 8 depicts oxycodone conjugated at the 6 position.

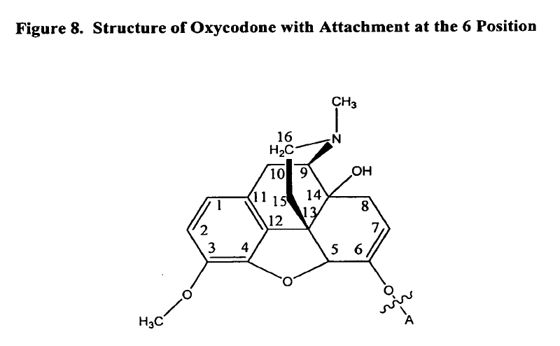

FIG. 9 depicts oxycodone conjugated at the 6 and 14 positions.

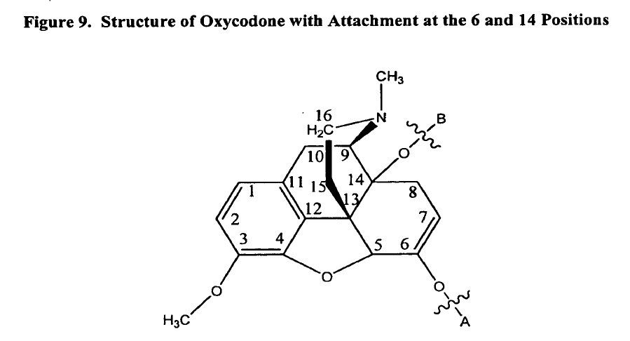

FIG. 10 depicts oxycodone conjugated at the 14 position.

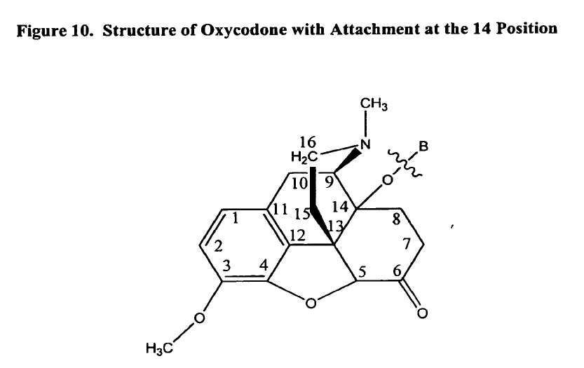

FIG. 11 depicts Oral Bioavailability of Disubstituted Peptide Oxycodone Compounds.

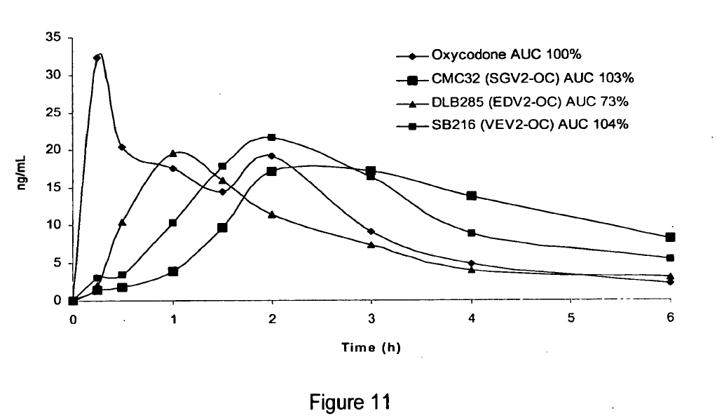

FIG. 12 depicts Oral Bioavailability of Monosubstituted Peptide Oxycodone Compounds.

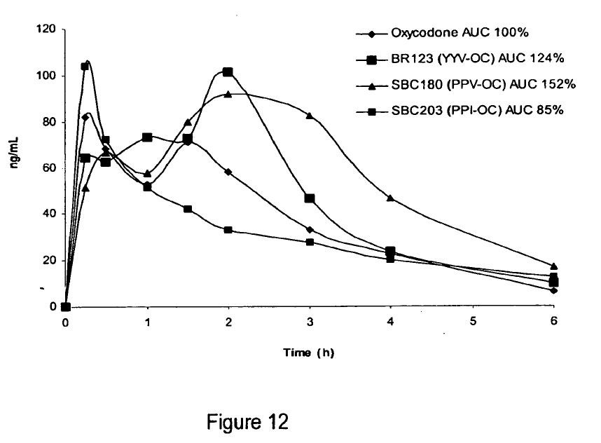

FIG. 13 depicts Oral Bioavailability of Non-Natural Single Amino Acid Oxycodone Compounds.

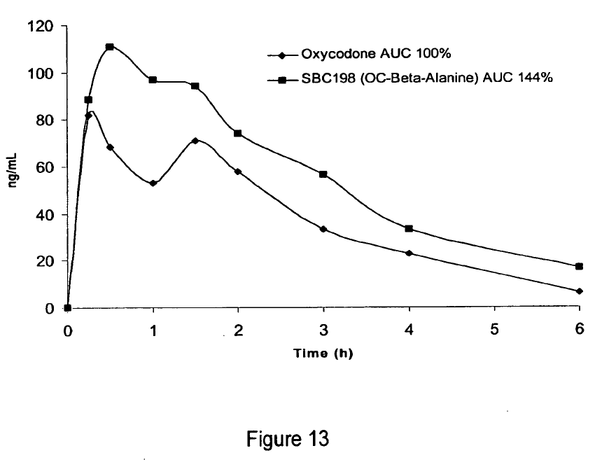

FIG. 14 depicts Intranasal Bioavailability of Disubstituted Peptide Oxycodone Compounds.

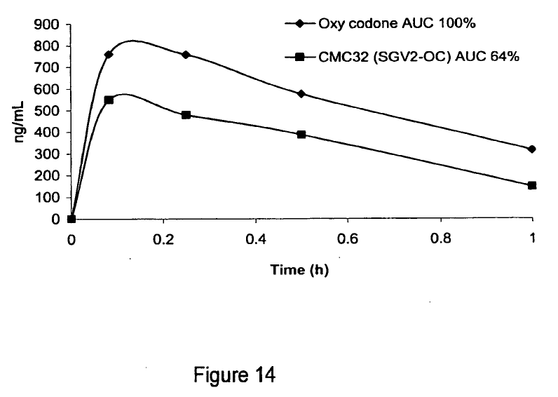

FIG. 15 depicts Intranasal Bioavailability of Disubstituted Peptide Oxycodone Compounds.

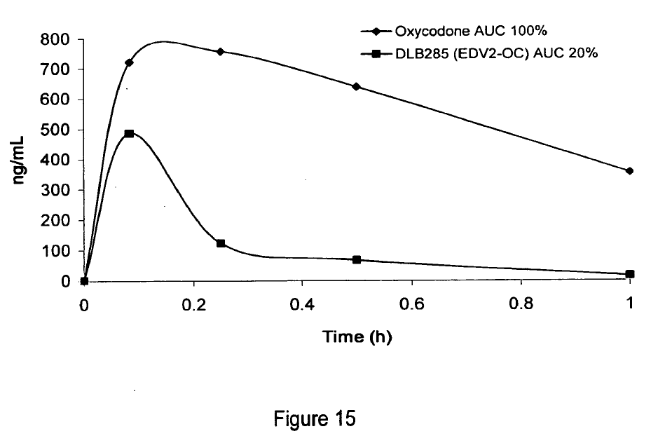

FIG. 16 depicts Intranasal Bioavailability of Disubstituted Peptide Oxycodone Compounds.

FIG. 17 depicts Intravenous Bioavailability of Disubstituted Peptide Oxycodone Compounds.

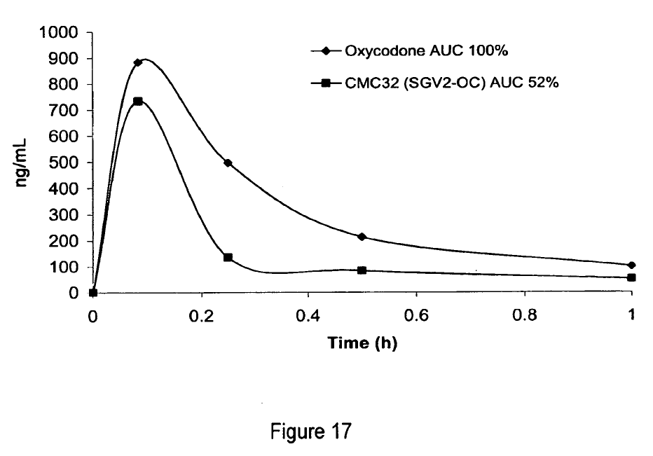

### DETAILED DESCRIPTION OF THE INVENTION

As used herein, the term “covalently bound compound” refers to a controlled substance that is covalently attached to a chemical moiety. Examples of covalently bound compounds include compounds that are described in U.S. patent application Ser. No. 10/156,527, filed on May 29, 2002, U.S. patent application Ser. No. 10/923,257, filed on Aug. 23, 2004, U.S. patent application Ser. No. 10/923,088, filed on Aug. 23, 2004, U.S. patent application Ser. No. 10/953,119, filed on Sep. 30, 2004 and U.S. patent application Ser. No. 10/953,110, filed on Sep. 30, 2004, all of which are hereby incorporated by reference in their entirety.

As used herein, the term “controlled substance” refers to analgesics including the following: alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, hydrocodone, hydromorphone, hydromorphodone, hydroxypethidine, isomethadone, ketobemidone, levorphanol, levophenacylmorphan, lofentanil, meperidine, meptazinol, metazocine, methadone, methyldihydromorphinone, metopon, morphine, myrophine, narceine, nicomorphine, norlevorphanol, normethadone, nalorphine, nalbuphene, normorphine, norpipanone, opium, oxycodone, oxymorphone, papavereturn, paregoric, pentazocine, phenadoxone, phendimetrazine, phendimetrazone, phenomorphan, phenazocine, phenoperidine, piminodine, piritramide, propheptazine, promedol, properidine, propoxyphene, propylhexedrine, sufentanil, sufentanyl, tilidine, tramadol, pharmaceutically acceptable salts thereof, and mixtures thereof, methylphenidate, barbiturates, benzodiazepines, skeletal muscle relaxants e.g., meprobamate, and stimulants including methylphenidate, pemoline, etc.

As used herein, the phrase “agonists that target a GPCR” means where the drug does not promote endocytosis and resensitization of the targeted GPCR include opioids such as morphine.

As used herein, the phrase “agonists that promotes the endocytosis of the GPCR” where the drug is present in an amount sufficient to promote endocystosis and resensitization of the targetted GPCR (mu opioid receptor agonist). Preferred drugs include methadone, fentanyl, sulfentanil, remi-fentanyl, etonitazene, and etorphine.

As used herein, the term “chemical moiety” refers to a substance made up of chemical elements and characterized by a defined molecular composition. It can exist as a part of the drug conjugate and can be separated from the conjugate. Examples include an amino acid, an oligopeptide or a polypeptide, but may be any number of other substances.

As used herein, the term “opioid” refers to compounds which bind to specific opioid receptors and have agonist (activation) or antagonist (inactivation) effects at these receptors, such as opioid alkaloids, including the agonist morphine and the antagonist naloxone, and opioid peptides, including enkephalins, dynorphins and endorphins.

A “covalently bound opioid” is an “opioid” that is covalently attached to a chemical moiety.

The term “opiate” refers to drugs derived from opium or related analogs.

As used herein, the term “Bimodally-acting opioid agonists” are opioid agonists that bind to and activate both inhibitory and excitatory opioid receptors on nociceptive neurons which mediate pain. Activation of inhibitory receptors by said agonists causes analgesia. Activation of excitatory receptors by said agonists results in anti-analgesia, hyperexcitability, hyperalgesia, as well as development of physical dependence, tolerance and other undesirable side effects.

The term “covalently bound bimodally-acting opioid agonists” are “bimodally-acting opioid agonists” that are covalently attached to a chemical moiety.

As used herein, the term “adverse side-effects” includes but is not limited to dizziness, nausea, constipation, headache, somnolence (sedation), vomiting, pruritis, CNS stimulation, seizures, asthenia, dyspepsia, diarrhea, physical dependence, dry mouth and/or sweating.

“Excitatory opioid receptor antagonists” are opioids which bind to and act as antagonists to excitatory but not inhibitory opioid receptors on nociceptive neurons which mediate pain. That is, excitatory opioid receptor antagonists are compounds which bind to excitatory opioid receptors and selectively block excitatory opioid receptor functions of nociceptive types of DRG neurons at 1,000 to 10,000-fold lower concentrations than are required to block inhibitory opioid receptor functions in these neurons.

The term “covalently bound opioid receptor antagonists” are “opioid receptor antagonists” that are covalently attached to a chemical moiety.

As used herein, an “analgesic” amount is amount of the covalently bound compound (or covalently bound bimodally-acting opioid agonist) which causes analgesia in a subject administered the covalently bound compound (or covalently bound bimodally-acting opioid agonist) alone, and includes standard doses of the compound or agonist which are typically administered to cause analgesia (e.g., mg doses).

A “sub-analgesic” amount is an amount which does not cause analgesia in a subject administered the covalently bound compound (or covalently bound bimodally-acting opioid agonist) alone, but when used in combination with the excitatory opioid receptor antagonist, results in analgesia.

Throughout this application the use of “chemical moiety”—sometimes referred to as the “conjugate” or the “carrier”—is meant to include any chemical substance, naturally occurring or synthetic that decreases the pharmacological activity until the active is released including at least carrier peptides, glycopeptides, carbohydrates, lipids, nucleic acids, nucleosides, or vitamins. Preferably, the chemical moiety is generally recognized as safe (“GRAS”).

Throughout this application the use of “carrier peptide” is meant to include naturally occurring amino acids, synthetic amino acids, and combinations thereof. In particular, carrier peptide is meant to include at least a single amino acid, a dipeptide, a tripeptide, an oligopeptide, a polypeptide, or the nucleic acid-amino acids peptides. The carrier peptide can comprise a homopolymer or heteropolymer of naturally occurring or synthetic amino acids.

The use of the term “straight carrier peptide” is meant to include amino acids that are linked via a —C(O)—NH— linkage, also referred to herein as a “peptide bond,” but may be substituted along the side chains of the carrier peptide. Amino acids that are not joined together via a peptide bond or are not exclusively joined through peptide bonds are not meant to fall within the definition of straight carrier peptide.

The use of the term “unsubstituted carrier peptide” is meant to include amino acids that are linked via a —C(O)—NH— linkage, and are not otherwise substituted along the side chains of the carrier peptide. Amino acids that are not joined together via a peptide bond or are not exclusively joined through peptide bonds are not meant to fall within the definition of unsubstituted carrier peptide.

“Oligopeptide” is meant to include from 2 amino acids to 10 amino acids. “Polypeptides” are meant to include from 11 to 50 amino acids.

“Carbohydrates” includes sugars, starches, cellulose, and related compounds. More specific examples include for instance, fructose, glucose, lactose, maltose, sucrose, glyceraldehyde, dihydroxyacetone, erythrose, ribose, ribulose, xylulose, galactose, mannose, sedoheptulose, neuraminic acid, dextrin, and glycogen.

A “glycoprotein” is a compound containing carbohydrate (or glycan) covalently linked to protein. The carbohydrate may be in the form of a monosaccharide, disaccharide(s). oligosaccharide(s), polysaccharide(s), or their derivatives (e.g. sulfo- or phosphosubstituted).

A “glycopeptide” is a compound consisting of carbohydrate linked to an oligopeptide composed of L- and/or D-amino acids. A glyco-amino-acid is a saccharide attached to a single amino acid by any kind of covalent bond. A glycosyl-amino-acid is a compound consisting of saccharide linked through a glycosyl linkage (O—, N— or S—) to an amino acid.

The “carrier range” or “carrier size” is determined based on the effect desired. It is preferably between one to 12 chemical moieties with one to 8 moieties being preferred. In another embodiment the number of chemical moieties attached is a specific number e.g., 1, 2, 3, 4, 5, 6, 7, 8, 9, or 10, etc. Alternatively, the chemical moiety may be described based on its molecular weight. It is preferred that the conjugate weight is below about 2,500 kD, more preferably below about 1,500 kD.

A “composition” as used herein, refers broadly to any composition containing a conjugate. A “pharmaceutical composition” refers to any composition containing a conjugate that only comprises components that are acceptable for pharmaceutical uses, e.g., excludes conjugates for immunological purposes.

Use of phrases such as “decreased”, “reduced”, “diminished”, or “lowered” includes at least a 10% change in pharmacological activity with respect to at least one ADME characteristic or at least one of AUC, Cₘₐₓ, Tₘₐₓ, Cₘᵢₙ, and t_{1/2} with greater percentage changes being preferred for reduction in abuse potential and overdose potential. For instance, the change may also be greater than 25%, 35%, 45%, 55%, 65%, 75%, 85%, 95%, 96%, 97%, 98%, 99%, or other increments.

Use of the phrase “similar pharmacological activity” means that two compounds exhibit curves that have substantially the same AUC, Cₘₐₓ, Tₘₐₓ, Cₘᵢₙ, and/or t_{1/2} parameters, preferably within about 30% of each other, more preferably within about 25%, 20%, 10%, 5%, 2%, 1%, or other increments.

“Cₘₐₓ” is defined as the maximum concentration of free active in the body obtained during the dosing interval.

“Tₘₐₓ” is defined as the time to maximum concentration.

“Cₘᵢₙ” is defined as the minimum concentration of active in the body after dosing.

“t_{1/2}” is defined as the time required for the amount of active in the body to be reduced to one half of its value.

Throughout this application, the term “increment” is used to define a numerical value in varying degrees of precision, e.g., to the nearest 10, 1, 0.1, 0.01, etc. The increment can be rounded to any measurable degree of precision. For example, the range 1 to 100 or increments therein includes ranges such as 20 to 80, 5 to 50, 0.4 to 98, and 0.04 to 98.05.

“Acute pain” is defined as sharp or severe pain or discomfort that lasts for a short period of time. Preferably, a short period of time is less than 3 months for nociceptive or neurogenic pain, and less than 6 months for psychogenic pain.

“Chronic pain” is defined as moderate to severe pain that lasts for a long period of time. Preferably, a long period of time is more than 3 months for nociceptive or neurogenic pain and more than 6 months for psychogenic pain.

“Patient” as used herein, refers broadly to any animal that is in need of treatment, most preferably and animal that is in pain. The patient may be a clinical patient such as a human or a veterinary patient such as a companion, domesticated, livestock, exotic, or zoo animal. Animals may be mammals, reptiles, birds, amphibians, or invertebrates.

“Mammal” as used herein, refers broadly to any and all warm-blooded vertebrate animals of the class Mammalia, including humans, non-human primates, felines, canines, pigs, horses, sheep, etc.

“Pretreatment” as used herein, refers broadly to any and all preparation, treatment, or protocol that takes place before receiving a hydromorphone compound or composition of the invention.

“Treating” or “treatment” as used herein, refers broadly to preventing the disease, i.e., causing the clinical symptoms of the disease not to develop in a patient that may be exposed to or predisposed to the disease but does not yet experience or display symptoms of the disease, inhibiting the disease, i.e., arresting or reducing the development of the disease or its clinical symptoms, and/or relieving the disease, i.e., causing regression of the disease or its clinical symptoms. Treatment also encompasses an alleviation of signs and/or symptoms.

“Therapeutically effective amount” as used herein, refers broadly to the amount of a compound that, when administered to a patient for treating pain is sufficient to effect such treatment for pain. The “therapeutically effective amount” will vary depending on the compound, the disease and its severity and the age, weight, etc., of the patient to be treated. “Effective dosage” or “Effective amount” of the active compound or composition is that which is necessary to treat or provide prophylaxis.

“Selection of patients” and “Screening of patients” as used herein, refers broadly to the practice of selecting appropriate patients to receive the treatments described herein. Various factors including but not limited to age, weight, heath history, medications, surgeries, injuries, conditions, illnesses, diseases, infections, gender, ethnicity, genetic markers, polymorphisms, skin color, and sensitivity to hydromorphone treatment. Still other factors include those used by physicians to determine if a patient is appropriate to receive the treatments described herein.

“Diagnosis” as used herein, refers broadly to the practice of testing, assessing, assaying, and determining whether or not a patient is in pain.

The invention is directed to compositions and methods for enhancing analgesic potency of controlled substances by providing sustained release of controlled substances, reducing the abuse potential of the controlled substances, and/or attenuating (e.g. reducing, blocking, inhibiting or preventing) adverse effects of controlled substances, particularly adverse side effects in humans through the covalent binding of at least one of a controlled substance, antagonist or agonist. Analgesic potency of the agonist may be maintained while one or more side effects are attenuated, without increasing or decreasing the cumulative daily dose of agonist. Compositions and methods of the invention thus solve the problem of a less than desired analgesic potency and/or adverse side effects associated with a covalently bound compound administration in humans. Further, the abuse potential of the covalently bound compound is reduced by preventing the release of the covalently bound compound and the covalently bound opioid antagonist when the compound is subjected to techniques employed by the illicit chemist.

The opioid prodrug compositions may prevent abuse by exhibiting reduced bioavailability due to the presence of the opioid antagonist when it is administered via parenteral routes, particularly the intravenous (“shooting”), intranasal (“snorting”), and/or inhalation (“smoking”) routes that are often employed in illicit use. Thus, the opioid prodrug may reduce the euphoric effect associated with opioid abuse when the opioid prodrug is used in a manner inconsistent with the manufacturer's instructions

The invention is directed to compositions for selectively enhancing the analgesic potency and simultaneously attenuating anti-analgesia, hyperalgesia, hyperexcitability, physical dependence and/or tolerance effects associated with the administration of a controlled substance (e.g. bimodally-acting opioid agonist). The composition comprises an analgesic or sub-analgesic amount of a covalently bound compound and an amount of an excitatory opioid receptor antagonist effective to enhance the analgesic potency of the covalently bound compound and attenuate the anti-analgesia, hyperalgesia, hyperexcitability, physical dependence and/or tolerance effects of the covalently bound compound, wherein said antagonist may or may not be covalently bound.

The invention also provides a composition of an analgesic or sub-analgesic amount of a covalently bound compound and an amount of a covalently bound opioid antagonist wherein if the composition is orally administered to a patient, the analgesic or sub-analgesic amount of the covalently bound compound is released, but the opioid antagonist is not. However, if the composition of an analgesic or sub-analgesic amount of a covalently bound compound and an amount of a covalently bound opioid antagonist is subjected to conditions likely to be employed by illicit chemists attempting to release the covalently bound compound, the covalently bound opioid antagonist is released, but the covalently bound compound is not released.

The invention also provides a composition of an analgesic or sub-analgesic amount of a covalently bound compound and an amount of a covalently bound opioid antagonist wherein if the composition is orally administered to a patient, the analgesic or sub-analgesic amount of the covalently bound compound is released, but the opioid antagonist is not. However, if the composition of an analgesic or sub-analgesic amount of a covalently bound compound and an amount of a covalently bound opioid antagonist is subjected to conditions likely to be employed by illicit chemists attempting to release the covalently bound compound, both the covalently bound compound and the covalently bound opioid antagonist are released.

The invention further provides a composition of an analgesic or sub-analgesic amount of a covalently bound compound and an amount of a covalently bound opioid antagonist effective to attenuate an adverse side effect of the covalently bound compound wherein if the composition is orally administered to a patient, both the analgesic or sub-analgesic amount of the covalently bound compound and the opioid antagonist are released. However, if the composition is subjected to conditions likely to be employed by illicit chemists attempting to release the covalently bound compound, only the covalently bound opioid antagonist is released.

The invention provides a composition of an analgesic or sub-analgesic amount of a covalently bound compound and an amount of a covalently bound opioid antagonist effective to attenuate an adverse side effect of the covalently bound compound wherein if the composition is orally administered to a patient, both the analgesic or sub-analgesic amount of the covalently bound compound and the opioid antagonist are released. However, if the composition is subjected to conditions likely to be employed by illicit chemists attempting to release the covalently bound compound, neither covalently bound compound, nor the covalently bound opioid antagonist is released.

The invention provides a composition of an analgesic or sub-analgesic amount of a covalently bound compound and an amount of opioid antagonist effective to attenuate an adverse side effect of the covalently bound compound wherein if the composition is orally administered to a patient, both the analgesic or sub-analgesic amount of the covalently bound compound and the opioid antagonist are released. However, if the composition is subjected to conditions likely to be employed by illicit chemists attempting to release the covalently bound compound, the covalently bound compound is not released, and the covalently bound opioid antagonist is released.

The invention is also directed to novel compositions comprising covalently bound agonists that target a GPCR, where the drug does not promote endocytosis and resensitization of the targeted GPCR and a covlently bound agonist that promotes the endocytosis of the GPCR and is present in the composition in an amount sufficient to promote endocystosis and resensitization of the targetted GPCR (mu opioid receptor agonist). The covalently bound agonists that target a GPCR, where the drug does not promote endocytosis and resensitization of the targeted GPCR includes an opioid drug such as morphine. The covalently bound mu opioid receptor agonist includes methadone, fentanyl, sulfentanil, remi-fentanyl, etonitazene, and etorphine.

The invention is also directed to novel compositions comprising covalently bound agonists that target a GPCR, where the drug does not promote endocytosis and resensitization of the targeted GPCR and a covlently bound agonist that promotes the endocytosis of the GPCR and is present in the composition in an amount sufficient to promote endocystosis and resensitization of the targetted GPCR (mu opioid receptor agonist) in combination with a covalently bound opioid antagonist.

The invention also provides a composition comprising a small dose of covalently bound or non-covalently bound racemic methadone in addition to covalently bound opioid agonist (such as morphine) so as to stimulate mu-opioid receptor (MOR) endocytosis and produce a decrease in constipation which normally occurs with high incidence when the agonist is given chronically on its own.

The invention also provides a method for treating pain by orally administering to the human subject any of the compositions as described above. The abuse potential is reduced by those compositions described above including an antagonist effective to block the euphoic effect of the covalently bound compound should the composition be subjected to techniques employed by the illicit chemist.

Thus, compositions containing covalently bound opioid and covalently bound opioid antagonist contemplated in the invention include the embodiments depicted in Table 1. Additional embodiments including another agonist may be formulated in accordance with the description as set forth in the specification.

TABLE 1
Compositions containing covalently bound opioid and covalently
bound or non-covalently bound opioid antagonist.

| | Release Characteristics | | | | Release Characteristics |
|---|---|---|---|---|---|
| | of Covalently Bound | | | | of Non-Covalently Bound |
| | antagonist | | | | Antagonist |
| Oral administration | | | | | |
| Opioid | R | R | R | R | R |
| Antagonist | NR | NR | R | R | R |
| Injection/Inhalation | | | | | |
| Opioid | NR | R | NR | NR | NR |
| Antagonist | R | R | R | NR | R |

R = Released, NR = Not Released

Exemplary compositions of the invention are also depicted in Table 2 below.

TABLE 2
Exemplary Compositions

| | Controlled Substance 1 | Controlled Substance 2 | Antagonist |
|---|---|---|---|
| Composition 1 | covalently bound | not present | covalently bound |
| | controlled substance | | antagonist |
| Composition 2 | covalently bound | not present | antagonist not |
| | controlled substance | | covalently bound |
| Composition 3 | controlled substance | not present | covalently bound |
| | not covalently bound | | antagonist |
| Composition 4 | covalently bound | covalently bound | not present |
| | controlled substance | controlled substance | |
| | that promotes | that does not promote | |
| | endocytosis | endocytosis | |
| Composition 5 | covalently bound | controlled substance | not present |
| | controlled substance | that does not promote | |
| | that promotes | endocytosis and is not | |
| | endocytosis | bound | |
| Composition 6 | controlled substance | covalently bound | not present |
| | that promotes | controlled substance | |
| | endocytosis and is | that does not promote | |
| | not bound | endocytosis | |
| Composition 7 | covalently bound | covalently bound | antagonist not |
| | controlled substance | controlled substance | covalently bound |
| | that promotes | that does not promote | |
| | endocytosis | endocytosis | |
| Composition 8 | covalently bound | controlled substance | antagonist not |
| | controlled substance | that does not promote | covalently bound |
| | that promotes | endocytosis and is not | |
| | endocytosis | bound | |
| Composition 9 | controlled substance | covalently bound | antagonist not |
| | that promotes | controlled substance | covalently bound |
| | endocytosis and is | that does not promote | |
| | not bound | endocytosis | |
| Composition 10 | covalently bound | covalently bound | covalently bound |
| | controlled substance | controlled substance | antagonist |
| | that promotes | that does not promote | |
| | endocytosis | endocytosis | |
| Composition 11 | covalently bound | controlled substance | covalently bound |
| | controlled substance | that does not promote | antagonist |
| | that promotes | endocytosis and is not | |
| | endocytosis | bound | |
| Composition 12 | controlled substance | covalently bound | covalently bound |
| | that promotes | controlled substance | antagonist |
| | endocytosis and is | that does not promote | |
| | not bound | endocytosis | |

The invention is directed to novel compositions and methods with covalently bound controlled substances and opioid antagonists. Combinations of covalently bound controlled substances and an opioid antagonist, such as naltrexone, can be efficacious in enhancing the analgesic potency of a covalently bound controlled substances and/or attenuating its side effects in humans. For example, potency may be enhanced at least about 2-fold by combination of a covalently bound compound and an opioid antagonist, so that the potency of a 50 mg dose of a covalently bound compound with a low dose (e.g., 0.01 mg) of antagonist (e.g., naltrexone) would be comparable to the potency of a 100 mg dose of a covalently bound compound alone.

The chemical moiety comprising the invention may be any chemical substance that can be attached to the controlled substance in a manner that renders it pharmacologically inactive. Analgesics produce their pharmacological effects through binding to specific receptors or uptake proteins. The attachment of certain chemical moieties can therefore prevent the active substance from binding its receptor(s) or recognition site on its uptake protein. Further, without being bound by theory, the covalent modification is believed to prevent the pharmacological effect by preventing the drug from crossing the blood-brain barrier. Preferably, the attachment of the chemical moiety to the controlled substance will also prevent or substantially delay the absorption of the compound, particularly when the compound is delivered by routes other than oral administration

The covalently bound controlled substances (or covalently bound bimodally-acting opioid agonist) and the excitatory opioid receptor antagonists (or covalently bound opioid receptor antagonists) may be formulated in compositions with a pharmaceutically acceptable carrier. The carrier must be “acceptable” in the sense of being compatible with the other ingredients of the formulation and not deleterious to the recipient thereof. The formulations may conveniently be presented in unit dosage and may be prepared by methods well-known in the pharmaceutical art, by bringing the active compound into association with a carrier or diluent, as a suspension or solution, and optionally one or more accessory ingredients, e.g. buffers, flavoring agents, surface active agents, and the like. The choice of carrier will depend upon the route of administration.

In another embodiment the invention provides a carrier and controlled substance which are bound to each other but otherwise unmodified in structure. This embodiment may further be described as the carrier having a free carboxy and/or amine terminal and/or side chain groups other than the location of attachment for the controlled substance. In a more preferred embodiment the carrier, whether a single amino acid, dipeptide, tripeptide, oligopeptide or polypeptide is comprised only naturally occurring amino acids.

The attached chemical moiety may be comprised of other naturally occurring or synthetic substances. Controlled substances, for example, could also be attached to lipids, carbohydrates, nucleic acids, or vitamins. These chemical moieties could be expected to serve the same functions as a peptide carrier such as delayed release in the gastrointestinal tract and/or prevention of rapid absorption of controlled substances.

The invention provides a composition comprising a peptide and an controlled substance covalently attached to the peptide. In another embodiment, the invention provides a composition comprising a peptide and an antagonist covalently attached to the peptide.

For each of the embodiments recited herein, the carrier peptide may comprise of one or more of the naturally occurring (L-)amino acids: alanine, arginine, asparagine, aspartic acid, cysteine, glycine, glutamic acid, glutamine, histidine, isoleucine, leucine, lysine, methionine, proline, phenylalanine, serine, tryptophan, threonine, tyrosine, and valine. Other preferred amino acids include beta-alanine, beta-leucine, and tertiary leucine. In another embodiment the amino acid or peptide is comprised of one or more of the D-form of the naturally occurring amino acids. In another embodiment the amino acid or peptide is comprised of one or more unnatural, non-standard or synthetic amino acids such as, aminohexanoic acid, biphenylalanine, cyclohexylalanine, cyclohexylglycine, diethylglycine, dipropylglycine, 2,3-diaminoproprionic acid, homophenylalanine, homoserine, homotyrosine, naphthylalanine, norleucine, ornithine, pheylalanine(4-fluoro), phenylalanine(2,3,4,5,6 pentafluoro), phenylalanine(4-nitro), phenylglycine, pipecolic acid, sarcosine, tetrahydroisoquinoline-3-carboxylic acid, and tert-leucine. In another embodiment the amino acid or peptide comprises of one or more amino acid alcohols. In another embodiment the amino acid or peptide comprises of one or more N-methyl amino acids.

In another embodiment, the specific carriers listed in the table may have one or more of amino acids substituted with one of the 20 naturally occurring amino acids. It is preferred that the substitution be with an amino acid which is similar in structure or charge compared to the amino acid in the sequence. For instance, isoleucine (Ile)[I] is structurally very similar to leucine (Leu)[L], whereas, tyrosine (Tyr)[Y] is similar to phenylalanine (Phe)[F], whereas serine (Ser)[S] is similar to threonine (Thr)[T], whereas cysteine (Cys)[C] is similar to methionine (Met)[M], whereas alanine (Ala)[A] is similar to valine (Val)[V], whereas lysine (Lys)[K] is similar to arginine (Arg)[R], whereas asparagine (Asn)[N] is similar to glutamine (Gln)[Q], whereas aspartic acid (Asp)[D] is similar to glutamic acid (Glu)[E], whereas histidine (His)[H] is similar to proline (Pro)[P], and glycine (Gly)[G] is similar to tryptophan (Trp)[W]. In the alternative the preferred amino acid substitutions may be selected according to hydrophilic properties (i.e., polarity) or other common characteristics associated with the essential amino acids. While preferred embodiments utilize the 20 natural amino acids for their GRAS characteristics, it is recognized that minor substitutions along the amino acid chain that do not affect the essential characteristics of the amino are also contemplated.

Covalent attachment of a chemical moiety to controlled substance may change one or more of the following: the rate of absorption, the extent of absorption, the metabolism, the distribution, and the elimination (ADME pharmacokinertic properties) of hydrocodone. As such, the alteration of one or more of these characteristics may be designed to provide fast or slow release depending of its use for chronic pain versus acute pain. Additionally, alteration of one or more of these characteristics may reduce the side effects associated with taking a controlled substance.

One aspect of the invention includes conjugates that when administered at a normal therapeutic dose the bioavailablility (area under the time-versus-concentration curve; AUC) of the controlled substance provides a pharmaceutically effective amount of the parent active. As the dose is increased, however, the bioavailability of the covalently modified controlled substance relative to the parent active begins to decline, particularly for oral dosage forms. At suprapharmacological doses the bioavailability of the controlled substance conjugate is substantially decreased as compared to the parent active. The relative decrease in bioavailability at higher doses decreases or reduces the euphoria obtained when doses of the controlled substance conjugate are taken above those of the intended prescription. This in turn diminishes the abuse potential, whether unintended or intentionally sought.

The compositions and methods of the invention provide actives, which when bound to the chemical moiety provide safer and/or more effective dosages for actives through improved bioavailability curves and/or safer Cₘₐₓ and/or reduce area under the curve for bioavailability, particularly for abused substances taken in doses above therapeutic levels. As a result, the compositions and methods of the invention may provide improved methods of treatment for analgesia.

Preferably, the prodrug exhibits an oral bioavailability of a controlled substance of at least about 60% AUC (area under the curve), more preferably at least about 70%, 80%, 90%, 95%, 96%, 97%, 98%, 99%, compared to unbound controlled substance, i.e. active. Preferably, the controlled substance prodrug exhibits a parenteral bioavailability, e.g., intranasal, bioavailability of less than about 70% AUC, more preferably less than about 50%, 30%, 20%, 15%, 10%, 5%, 4%, 3%, 2%, 1%, compared to unbound controlled substance.

In one embodiment, the controlled substance prodrug provides pharmacological parameters (AUC, Cₘₐₓ, Tₘₐₓ, Cₘᵢₙ, and/or t_{1/2}) within 80% to 125%, 80% to 120%, 85% to 125%, 90% to 110%, or increments therein of unbound hydrocodone. It should be recognized that the ranges can, but need not be symmetrical, e.g., 85% to 105%.

In another embodiment, the toxicity of the controlled substance prodrug is substantially lower than that of the unbound controlled substance. For example, in a preferred embodiment, the acute toxicity is 1-fold, 2-fold, 3-fold, 4-fold, 5-fold, 6-fold, 7-fold, 8-fold, 9-fold, 10-fold less, or increments therein less lethal than oral administration of unbound controlled substance.

For each of the described embodiments one or more characteristics as described throughout the specification may be realized. It should also be recognized that the compounds and compositions described throughout the specification may be utilized for a variety of novel methods of treatment, reduction of abuse potential, reduction of toxicity, improved release profiles, etc. An embodiment may obtain, one or more of: a conjugate with toxicity of that is substantially lower than that of an unbound controlled substance; a conjugate where the covalently bound chemical moiety reduces or eliminates the possibility of overdose by oral administration; a conjugate where the covalently bound chemical moiety reduces or eliminates the possibility of overdose by intranasal administration; and/or a conjugate where the covalently bound chemical moiety reduces or eliminates the possibility of overdose by injection.

The conjugates may also be in salt form. Pharmaceutically acceptable salts, e.g., non-toxic, inorganic and organic acid addition salts, are known in the art. Exemplary salts include, but are not limited to, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, bisulfate, bitartrate, borate, butyrate, calcium edetate, camphorate, camphorsulfonate, citrate, clavulariate, cyclopentanepropionate, digluconate, dodecylsulfate, edetate, edisylate, estolate, esylate, ethanesulfonate, finnarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, isothionate, lactate, lactobionate, laurate, laurylsulphonate, malate, maleate, mandelate, methanesulfonate, mucate, naphthylate, napsylate, nicotinate, N-methylglucamine ammonium salt, oleate, palmitate, pamoate, pantothenate, pectinate, phosphate, phosphateldiphosphate, pivalate, polygalacturonate, propionate, p-toluenesulfonate, saccharate, salicylate, stearate, subacetate, succinate, sulfate, sulfosaliculate, suramate, tannate, tartrate, teoclate, tosylate, triethiodide, undecanoate, and valerate salts, and the like.

In the invention, the controlled substance may be covalently attached to the peptide via the ketone group and a linker. This linker may be a small linear or cyclic molecule containing 2-6 atoms with one or more heteroatoms (such as O, S, N) and one or more functional groups (such as amines, amides, alcohols or acids) or may be made up of a short chain of either amino acids or carbohydrates). For example, glucose would be suitable as a linker. The linker should have a functional pendant group, such as a carboxylate, an alcohol, thiol, oxime, hydraxone, hydrazide, or an amine group, to covalently attach to the carrier peptide.

The invention provides covalent attachment of controlled substances or antagonists to a peptide. It is preferred that aside from attachment of the carrier peptide to the hydrocodone neither is further substituted or protected. In one embodiment, the chemical moiety has one or more free carboxy and/or amine terminal and/or side chain group other than the point of attachment to the hydrocodone. The chemical moiety can be in such a free state, or an ester or salt thereof.

In another embodiment of the invention, the analgesic or antagonist is attached to an oligopeptide, preferably consisting of between one and five amino acids. In a further embodiment of the invention the amino acids are a heterogeneous mixture of the twenty naturally occurring amino acids. Hydrophilic amino acids will tend to prevent passive absorption of the analgesic peptide conjugate through nasal membranes. Thus it is a preferred embodiment of the invention that hydrophilic amino acids be included in the oligopeptide. It is a further preferred embodiment of the invention that lipophilic amino acids be attached closer to the analgesic for optimum stability. Both lipophilic and hydrophilic properties (i.e., amphiphilic) can be satisfied with between three and five amino acids. Thus it is a more preferred embodiment of the invention that the oligopeptide that is attached to the analgesic or antagonist be an amphiphilic tripeptide.

Preferred amphiphilic amino acids/oligopeptides may be selected from (i) hydrophobic amino acids, preferably in positions next to the controlled substance to provide increased stability; (ii) amino acid sequences designed to be cleaved by intestinal enzymes (e.g., pepsin, trypsin, chymotrypsin, elastase, carboxypeptidases A and B, etc.) provide for increased bioavailability; (iii) peptides longer than three amino acids for increased stability, increased anti-abuse e.g. less membrane permeability, and potentially more efficient intestinal digestion e.g. major intestinal enzymes target proteins and polypeptides, (iv) or mixtures thereof. In one preferred embodiment the carrier portion of the conjugate is designed for intestinal cleavage.

Suitable covalently bound bimodally-acting opioid agonists include but are not limited to morphine, codeine, fentanyl analogs, pentazocine, buprenorphine, methadone, enkephalins, dynorphins, endorphins and similarly acting opioid alkaloids and opioid peptides that are covalently attached to a chemical moiety. For purposes of treating pain, hydrocodone, hydromorphone, oxycodone, methadone, fentanyl, morphine and codeine covalently attached to a chemical moiety are preferred.

Suitable excitatory opioid receptor antagonists of the invention include, but are not limited to nalmefene, naltrexone, naloxone, etorphine and dihydroetorphine, as well as similarly acting opioid alkaloids, opioid peptides and mixtures thereof. Preferred excitatory opioid receptor antagonists are nalmefene and naltrexone because of their longer duration of action as compared to naloxone and their greater bioavailability after oral administration. The above mentioned excitatory opioid receptor antagonists are also suitable as the antagonists present in the covalently bound opioid receptor antagonists.

The covalently bound controlled substances (or covalently bound bimodally-acting opioid agonists) and the excitatory opioid receptor antagonists (or covalently bound opioid receptor antagonists) for use in the invention may be in the form of free bases or pharmaceutically acceptable acid addition salts thereof. Examples of suitable acids for salt formation include but are not limited to methanesulfonic, sulfuric, hydrochloric, glucuronic, phosphoric, acetic, citric, lactic, ascorbic, maleic, and the like.

When the covalently bound opioid receptor antagonists are employed to block the inhibitory opioid receptor functions and thus the euphoric effect of the covalently bound compounds, coventional dosage amounts are applied for this purpose and are known to one of ordinary skill in the art. For example, a dosage amount equivalent to 50 mg per day is sufficient to accomplish this purpose.

The excitatory opioid receptor antagonist (or covalently bound opioid receptor antagonists) alone, or in combination with the covalently bound compound (or covalently bound bimodally-acting opioid agonist), may be administered to a human or animal subject by known procedures, e.g., sublingual, oral, or transdermal. When a combination of these compounds are administered, they may be administered together in the same composition, or may be administered in separate compositions. If the covalently bound compound (or covalently bound bimodally-acting opioid agonist) and the excitatory opioid receptor antagonist (or covalently bound opioid receptor antagonists) are administered in separate compositions, they may be administered by similar or different modes of administration, and may be administered simultaneously with one another, or shortly before or after the other.

When the excitatory opioid receptor antagonist (or covalently bound opioid receptor antagonists) is used in combination with the covalently bound compound (or covalently bound bimodally-acting opioid agonist), the amount of the covalently bound compound (or covalently bound bimodally-acting opioid agonist) administered may be an analgesic or sub-analgesic amount. The amount of the excitatory opioid receptor antagonist (or covalently bound opioid receptor antagonists) is an amount effective to enhance the analgesic potency of the covalently bound compound (or covalently bound bimodally-acting opioid agonist) and attenuate the anti-analgesia, hyperalgesia, hyperexcitability, physical dependence and/or tolerance effects of the covalently bound compound (or covalently bound bimodally-acting opioid agonist). The amount of the excitatory opioid receptor antagonist (or covalently bound opioid receptor antagonists) administered may be between about 1000 and about 10,000,000 fold less, and preferably between about 10,000 and 1,000,000 fold less than the amount of the covalently bound compound (or covalently bound bimodally-acting opioid agonist) administered. The optimum amounts of the covalently bound compound (or covalently bound bimodally-acting opioid agonist) and the excitatory opioid receptor antagonist (or covalently bound opioid receptor antagonists) administered will of course depend upon the particular agonist and antagonist used, the carrier chosen, the route of administration, and the pharmacokinetic properties of the subject being treated.

When the excitatory opioid receptor antagonist (or covalently bound opioid receptor antagonists) is administered alone (i.e., for treating an opioid addict), the amount of the excitatory opioid receptor antagonist administered is an amount effective to attenuate physical dependence caused by a covalently bound compound (or covalently bound bimodally-acting opioid agonist) such as morphine (attached to a chemical moiety) and enhance the analgesic potency of the covalently bound compound (or covalently bound bimodally-acting opioid agonist). That is, the amount of the excitatory opioid receptor antagonist is an amount which blocks the excitatory effects (e.g., physical dependence) of the covalently bound compound (or covalently bound bimodally-acting opioid agonist) without blocking the inhibitory effects (i.e., analgesic effects) of the covalently bound compound (or covalently bound bimodally-acting opioid agonist). This amount is readily determinable by one skilled in the art.

When a covalently bound agonists that target a GPCR, where the drug does not promote endocytosis and resensitization of the targeted GPCR and a covlently bound agonist that promotes the endocytosis of the GPCR and is present in the composition in an amount sufficient to promote endocystosis and resensitization of the targetted GPCR (mu opioid receptor agonist) are present, it is not necessary that the mu opioid receptor agonist be present in sufficient amounts to produce any physiological or pharmacological effect of its own other than to promote endocytosis of the receptor. In this vein, it is desirable that the amount of the agonist that is co-administered be as low as possible to minimize any side effects attributable to the activity of the agonist, while still being an amount of agonist sufficient to promote receptor endocystosis. In general, for both the drug and the agonist, the determination of suitable dosing regimens are within the competence of one of ordinary skill in the medical arts and may be found with reference to the manufacturer's or supplier's instructions, or The Physicians's Desk Reference.

When methadone is used in conjunction with a covalently bound opioid in order to produce a decrease in constipation, the amount of methadone administered is an amount effective to accomplish this purpose. This amount is typically between 10 and 10⁸ fold less than the covalently bound opioid. Preferably, the amount of methadone is 10² to 10⁶ fold less than the covalently bound opioid. More preferably, the amount of methadone is 10³ to 10⁵ fold less than the covalently bound opioid.

In one embodiment, the covalently attached chemical moiety is removed by the acidic content of the stomach if the controlled substance is attached through an acid labile bond. More preferably, the covalently attached chemical moiety can be removed by enzymatic activity encountered by the compound in the stomach and/or intestinal tract. The stomach and intestinal tract are bathed in degradative enzymes. For example, the pancreas releases into the small intestine a myriad of hydrolytic enzymes such as proteases, lipases, and amylases, and nucleases. Additionally, the intestinal epithelial cells that line the surface of the GI tract produce various surface associated and intracellular degradative enzymes (e.g. brush border peptidases, esterases). These enzymes degrade proteins, lipids, carbohydrates, and nucleic acids contained in ingested food. Thus, it can be expected that the controlled substance will be released from the attached chemical moiety when the appropriate enzyme(s) is encountered in the gastrointestinal tract.

In another embodiment of the invention, the chemical moiety is attached to the controlled substance (or agonist) or opioid receptor antagonists in a manner in which it is not readily released by conditions found in the mouth (saliva), the intranasal cavity, the surface of the lungs, or in the serum. Extreme acid conditions encountered in the stomach are not present elsewhere in humans. Therefore, any acid dependent release mechanism will occur only after oral administration. Although, degradative enzymes are present in the aforementioned environments, they are not generally present in the high concentrations found in the intestinal tract. Thus, release of the controlled substance by enzymatic cleavage will not occur rapidly when the novel compounds are administered by routes other than oral delivery.

In another embodiment of the invention, the analgesic (e.g., oxycodone, hydrocodone, etc.) or antagonist (e.g. naltrexone) are attached to a polymer of serine (or other amino acid containing a hydroxyl side chain e.g. threonine, tyrosine) via side chain hydroxyl groups. Alternatively, attachment is to a polymer of glutamic acid through the carboxyl group of the delta carbon of glutamic acid. The resulting ester (carbonate) linkages can be hydrolysed by lipases (esterases) encountered in the small intestine. Esterases are not present at high levels in saliva or on the mucosal surfaces of the nasal cavity, lungs, or oral cavity. Thus, controlled substances or antagonists attached to polyglutamic acid by this method would not be rapidly released by saliva or when delivered intranasally or by inhalation.

In another embodiment, the invention also provides a method for delivering a controlled substance to a patient, the patient being a human or a non-human animal, comprising administering to the patient a composition comprising a peptide and an controlled substance covalently attached to the peptide. In a preferred embodiment, the controlled substance or antagonist are released from the composition by enzyme catalysis. In another preferred embodiment, the controlled substance or antagonist are released in a time-dependent manner based on the pharmacokinetics of the enzyme-catalyzed release.

One embodiment of the invention relates to long acting controlled substances having significantly reduced abuse potential. The controlled substance is covalently bound to a peptide/oligopeptide or amino acid, which renders the controlled substance pharmaceutically inactive until released following oral administration. Preferably the release mechanism is enzymatic action. The enzymatic and/or chemical conditions necessary for the release of the controlled substances is either not present or is minimally active when the drug-peptide conjugate is introduced by inhalation or injection. Thus it is expected that no euphoric effect will occur when the drug-peptide conjugate is inhaled or injected. Further, extending the release of the controlled substance prevents spiking of drug levels which provide the desired analgesic effect with a lower or absent euphoria. Controlled substances with these novel properties are less likely to be abused due to the diminished “rush” effect of the modified controlled substance. Consequently, decreasing euphoria while increasing the duration of the analgesic effect enhances and reducing the likelihood of abuse increases the therapeutic value of these pharmaceuticals. The invention further provides for administering to a subject an analgesic or sub-analgesic amount of the drug-peptide conjugates and an amount of an excitatory opioid receptor antagonist effective to enhance the analgesic potency of the drug-peptide cojugates and attenuate the anti-analgesia, hyperalgesia, hyperexcitability, physical dependence and/or tolerance effects of the covalently bound compound. The invention also provides for reproducible methods for compositions that are abuse-free for controlled substances, stable under a variety of chemical conditions, reduced euphoric effect and extended absorption into the bloodstream.

In addition to the prodrug, the pharmaceutical compositions of the invention may further comprise one or more pharmaceutical additives. Pharmaceutical additives include a wide range of materials including, but not limited to diluents and bulking substances, binders and adhesives, lubricants, glidants, plasticizers, disintegrants, carrier solvents, buffers, colorants, flavorings, sweeteners, preservatives and stabilizers, adsorbents, and other pharmaceutical additives known in the art.

Lubricants include, but are not limited to, magnesium stearate, calcium stearate, zinc stearate, powdered stearic acid, glyceryl monostearate, glyceryl palmitostearate, glyceryl behenate, silica, magnesium silicate, colloidal silicon dioxide, titanium dioxide, sodium benzoate, sodium lauryl sulfate, sodium stearyl fumarate, hydrogenated vegetable oil, talc, polyethylene glycol, and mineral oil.

Surface agents for formulation include, but are not limited to, sodium lauryl sulfate, dioctyl sodium sulfosuccinate, triethanolamine, polyoxyethylene sorbitan, poloxalkol, and quarternary ammonium salts; excipients such as lactose, mannitol, glucose, fructose, xylose, galactose, sucrose, maltose, xylitol, sorbitol, chloride, sulfate and phosphate salts of potassium, sodium, and magnesium; gelling agents such as colloidal clays; thickening agents such as gum tragacanth or sodium alginate, effervescing mixtures; and wetting agents such as lecithin, polysorbates or laurylsulphates.

Colorants can be used to improve appearance or to help identify the pharmaceutical composition. See 21 C.F.R., Part 74. Exemplary colorants include D&C Red No. 28, D&C Yellow No. 10, FD&C Blue No. 1, FD&C Red No. 40, FD&C Green #3, FD&C Yellow No. 6, and edible inks.

In embodiments where the pharmaceutical composition is compacted into a solid dosage form, e.g., a tablet, a binder can help the ingredients hold together. Binders include, but are not limited to, sugars such as sucrose, lactose, and glucose; corn syrup; soy polysaccharide, gelatin; povidone (e.g., Kollidon®, Plasdone®); Pullulan; cellulose derivatives such as microcrystalline cellulose, hydroxypropylmethyl cellulose (e.g., Methocel®), hydroxypropyl cellulose (e.g., Klucel®), ethylcellulose, hydroxyethyl cellulose, carboxymethylcellulose sodium, and methylcellulose; acrylic and methacrylic acid co-polymers; carbomer (e.g., Carbopol®); polyvinylpolypyrrolidine, polyethylene glycol (Carbowax®); pharmaceutical glaze; alginates such as alginic acid and sodium alginate; gums such as acacia, guar gum, and arabic gums; tragacanth; dextrin and maltodextrin; milk derivatives such as whey; starches such as pregelatinized starch and starch paste; hydrogenated vegetable oil; and magnesium aluminum silicate, as well as other conventional binders known to persons skilled in the art. Exemplary non-limiting bulking substances include sugar, lactose, gelatin, starch, and silicon dioxide.

Glidants can improve the flowability of non-compacted solid dosage forms and can improve the accuracy of dosing. Glidants include, but are not limited to, colloidal silicon dioxide, fumed silicon dioxide, silica gel, talc, magnesium trisilicate, magnesium or calcium stearate, powdered cellulose, starch, and tribasic calcium phosphate.

Plasticizers include, but are not limited to, hydrophobic and/or hydrophilic plasticizers such as, diethyl phthalate, butyl phthalate, diethyl sebacate, dibutyl sebacate, triethyl citrate, acetyltriethyl citrate, acetyltributyl citrate, cronotic acid, propylene glycol, castor oil, triacetin, polyethylene glycol, propylene glycol, glycerin, and sorbitol. Plasticizers are particularly useful for pharmaceutical compositions containing a polymer and in soft capsules and film-coated tablets.

Flavorings improve palatability and may be particularly useful for chewable tablet or liquid dosage forms. Flavorings include, but are not limited to maltol, vanillin, ethyl vanillin, menthol, citric acid, fumaric acid, ethyl maltol, and tartaric acid. Sweeteners include, but are not limited to, sorbitol, saccharin, sodium saccharin, sucrose, aspartame, fructose, mannitol, and invert sugar.

Preservatives and/or stabilizers improving storagability include, but are not limited to, alcohol, sodium benzoate, butylated hydroxy toluene, butylated hydroxyanisole, and ethylenediamine tetraacetic acid.

Disintegrants can increase the dissolution rate of a pharmaceutical composition. Disintegrants include, but are not limited to, alginates such as alginic acid and sodium alginate, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), polyvinylpolypyrrolidine (Plasone-XL®), guar gum, magnesium aluminum silicate, methyl cellulose, microcrystalline cellulose, polacrilin potassium, powdered cellulose, starch, pregelatinized starch, sodium starch glycolate (e.g., Explotab®, Primogel®).

Diluents increase the bulk of a dosage form and may make the dosage form easier to handle. Exemplary diluents include, but are not limited to, lactose, dextrose, saccharose, cellulose, starch, and calcium phosphate for solid dosage forms, e.g., tablets and capsules; olive oil and ethyl oleate for soft capsules; water and vegetable oil for liquid dosage forms, e.g., suspensions and emulsions. Additional suitable diluents include, but are not limited to, sucrose, dextrates, dextrin, maltodextrin, microcrystalline cellulose (e.g., Avicel®), micro fine cellulose, powdered cellulose, pregelatinized starch (e.g., Starch 15000), calcium phosphate dihydrate, soy polysaccharide (e.g., Emcosoy®), gelatin, silicon dioxide, calcium sulfate, calcium carbonate, magnesium carbonate, magnesium oxide, sorbitol, mannitol, kaolin, polymethacrylates (e.g., Eudragit®), potassium chloride, sodium chloride, and talc.

In embodiments where the pharmaceutical composition is formulated for a liquid dosage form, the pharmaceutical composition may include one or more solvents. Suitable solvents include, but are not limited to, water; alcohols such as ethanol and isopropyl alcohol; vegetable oil; polyethylene glycol; propylene glycol; and glycerin or mixing and combination thereof.

The pharmaceutical composition can comprise a buffer. Buffers include, but are not limited to, lactic acid, citric acid, acetic acid, sodium lactate, sodium citrate, and sodium acetate.

Hydrophilic polymers suitable for use in the sustained release formulation include: one or more natural or partially or totally synthetic hydrophilic gums such as acacia, gum tragacanth, locust bean gum, guar gum, or karaya gum, modified cellulosic substances such as methylcellulose, hydroxomethylcellulose, hydroxypropyl methylcellulose, hydroxypropyl cellulose, hydroxyethylcellulose, carboxymethylcellulose; proteinaceous substances such as agar, pectin, carrageen, and alginates; and other hydrophilic polymers such as carboxypolymethylene, gelatin, casein, zein, bentonite, magnesium aluminum silicate, polysaccharides, modified starch derivatives, and other hydrophilic polymers known to those of skill in the art or a combination of such polymers.

One of ordinary skill in the art would recognize a variety of structures, such as bead constructions and coatings, useful for achieving particular release profiles. It is also possible for the dosage form to combine any forms of release known to persons of ordinary skill in the art. These include immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is known in the art. See, e.g., U.S. Pat. No. 6,913,768.

However, it should be noted that the opioid conjugate controls the release of opioid into the digestive tract over an extended period of time resulting in an improved profile when compared to immediate release combinations and reduces and/or prevents abuse without the addition of the above additives. In a preferred embodiment no further sustained release additives are required to achieve a blunted or reduced pharmacokinetic curve (e.g. reduced euphoric effect) while achieving therapeutically effective amounts of opioid release.

The dose range for adult human beings will depend on a number of factors including the age, weight and condition of the patient and the administration route. Tablets and other forms of presentation provided in discrete units conveniently contain a daily dose, or an appropriate fraction thereof, of the opioid conjugate. The dosage form can contain a dose of about 2.5 mg to about 500 mg, about 10 mg to about 250 mg, about 10 mg to about 100 mg, about 25 mg to about 75 mg, or increments therein. In a preferred embodiment, the dosage form contains 5 mg, 7.5 mg, 10 mg, 12 mg, 18 mg, 24 mg, 30 mg, or 50 mg of an opioid prodrug.

Tablets and other dosage forms provided in discrete units can contain a daily dose, or an appropriate fraction thereof, of one or more opioid prodrugs.

Compositions of the invention may be administered in a partial, i.e., fractional dose, one or more times during a 24 hour period, a single dose during a 24 hour period of time, a double dose during a 24 hour period of time, or more than a double dose during a 24 hour period of time. Fractional, double or other multiple doses may be taken simultaneously or at different times during the 24-hour period. The doses may be uneven doses with regard to one another or with regard to the individual components at different administration times. Preferably, a single dose is administered once daily.

Likewise, the compositions of the invention may be provided in a blister pack or other such pharmaceutical package. Further, the compositions of the present inventive subject matter may further include or be accompanied by indicia allowing individuals to identify the compositions as products for a prescribed treatment. The indicia may further additionally include an indication of the above specified time periods for administering the compositions. For example the indicia may be time indicia indicating a specific or general time of day for administration of the composition, or the indicia may be a day indicia indicating a day of the week for administration of the composition. The blister pack or other combination package may also include a second pharmaceutical product.

The compounds of the invention can be administered by a variety of dosage forms. Any biologically acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspension in an aqueous liquid or a non-aqueous liquid, emulsions, tablets, syringes, multi-layer tablets, bi-layer tablets, capsules, soft gelatin capsules, hard gelatin capsules, caplets, lozenges, chewable lozenges, beads, powders, granules, particles, microparticles, dispersible granules, cachets, and combinations thereof. Preferably, said composition may be in the form of any of the known varieties of tablets (e.g., chewable tablets, conventional tablets, film-coated tablets, compressed tablets), capsules, liquid dispersions for oral administration (e.g., syrups, emulsions, solutions or suspensions).

However, the most effective means for delivering the abuse-resistant opioid compounds of the invention is orally, to permit maximum release of opioid to provide therapeutic effectiveness and/or sustained release while maintaining abuse resistance. When delivered by the oral route opioid is released into circulation, preferably over an extended period of time as compared to active alone.

It is preferred that the conjugate be compact enough to allow for a reduction in overall administration size. The smaller size of the prodrug dosage forms promotes ease of swallowing.

For oral administration, fine powders or granules containing diluting, dispersing and/or surface-active agents may be presented in a draught, in water or a syrup, in capsules or sachets in the dry state, in a non-aqueous suspension wherein suspending agents may be included, or in a suspension in water or a syrup. Where desirable or necessary, flavoring, preserving, suspending, thickening or emulsifying agents can be included.

Accordingly, the invention also provides methods comprising providing, administering, prescribing, or consuming a prodrug. The invention also provides pharmaceutical compositions comprising prodrug. The formulation of such a pharmaceutical composition can optionally enhance or achieve the desired release profile.

### EXAMPLES

The examples demonstrate the use and effectiveness of an chemical moiety conjugated to a controlled substance for reducing the potential for overdose while maintaining its therapeutic value including providing sustain release and/or reduced side effects. Further, these compounds can be utilized in compositions and methods described above to wherein the controlled substance is used in combination with an antagonist, a second agonist (controlled substance) or a combination thereof. However, it should be recognized that the below examples are illustrative only and that other controlled substances or antagonist could be utilized to achieve the desired results of the invention described throughout the specification. Additional examples of hydrocodone conjugates that are useful in the invention may be found for instance in US 2005/0266070 A1, which is herein incorporated by reference in its entirety. Additional examples of oxycodone conjugates that are useful in the invention may be found for instance in US 2005/0176644 A1, which is herein incorporated by reference in its entirety.

TABLE 3
List of Preferred Amino Acids and Peptides to
which Opioids may be covalently bonded.

(Lys-Lys-Gly-Gly)₂
[(1)-Lys-(d)-Lys-Leu]₂
Acetyl-Glu₂-Pro₂-Ile [SEQ ID NO: 3]
Ala
Ala-Pro
aminoisobutyric acid
Arg
Asn
Asp
Asp₂-Gly₂-Ile [SEQ ID NO: 4]
Asp₂-Leu₂-Ile [SEQ ID NO: 5]
Asp₂-Leu₂-Ile [SEQ ID NO: 6]
Asp₂-Lys(Asp₂) [SEQ ID NO: 29]
Asp₂-Pro₂-Ile [SEQ ID NO: 7]
Asp-Asp-Cha
Asp-Asp-Ile
Asp-Asp-Nle
Asp-Asp-Phe
Asp-Asp-Val
Asp-d-Asp-Ile
Asp-Glu-Val
Asp-Gly-Val
Asp-Ile-Val
Asp-Leu-Val
Asp-Lys-Val
Asp-Phe-Val
Asp-Pro-Val
Asp-Ser-Val
Asp-Thr-Val
Asp-Tyr-Val
Asp-Val-Val
Cys
Ethyl Carbonate
galactose-Gly-Gly-Ile
galactose-Gly-Gly-Leu
galactose-Ile
Gln
Gln-Gln-Ile
Gln-Gln-Val
Gln-Gln-β-Ala
Gln-Pro-Val
Glu
Glu₂-Gly₂-Phe [SEQ ID NO: 8]
Glu₂-Leu_{3 [SEQ ID NO: 9]}
Glu₂-Lys(Glu₂) [SEQ ID NO: 30]
Glu₂-Phe₂-Leu [SEQ ID NO: 10]
Glu₂-Phe-Pro-Ile [SEQ ID NO: 11]
Glu₂-Pro₂-Leu [SEQ ID NO: 12]
Glu₂-Pro-Phe-Ile [SEQ ID NO: 13]
Glu₄-Ile [SEQ ID NO: 14]
Glu₅
Glu-Glu
Glu-Glu-Cha
Glu-Glu-Glu
Glu-Glu-Gly-Gly-Aib
Glu-Glu-Gly-Gly-Ile
Glu-Glu-Gly-Gly-Leu
Glu-Glu-hPhe
Glu-Glu-Ile
Glu-Glu-Leu
Glu-Glu-Nle
Glu-Glu-Phe
Glu-Glu-Phe-Phe-Ile
Glu-Glu-Phe-Phe-Phe [SEQ ID NO: 15]
Glu-Glu-Val
Glu-Gly-Val
Glu-Leu-Val
Glu-Lys-Val
Glu-Phe-Val
Glu-Pro-Val
Glu_{pyro}-Glu
Glu-Ser-Val
Glu-Thr-Val
Glu-Tyr-Val
Glu-Val-Val
Gly
Gly₂-Glu₂-Ile [SEQ ID NO: 16]
Gly₂-Glu₃
Gly₄-Aib
Gly₄-Ile
Gly₄-Leu
Gly₄-Phe
Gly-Asp-Val
Gly-Gly-alpha aminoisobutyric acid
Gly-Gly-Cha
Gly-Gly-Glu
Gly-Gly-hPhe
Gly-Gly-Ile
Gly-Gly-Leu
Gly-Gly-Nle
Gly-Gly-Phe
Gly-Gly-Val
Gly-Ile-Ile
Gly-Leu-Ile
Gly-Leu-Leu
Gly-Lys-Val
Gly-Phe-Ile
Gly-Phe-Leu
Gly-Pro-Val
Gly-Ser-Val
Gly-Thr-Val
Gly-Val-Val
His
Ile
Ile-Asp-Val
Ile-Glu-Val
Ile-Gly-Val
Ile-Phe-Val
Ile-Ser-Val
Ile-Thr-Val
Ile-Tyr-Val
Leu
Leu-Asp-Val
Leu-Glu-Val
Leu-Gly-Val
Leu-Leu-Glu
Leu-Leu-Ile
Leu-Leu-Leu
Leu-Lys-Val
Leu-Phe-Val
Leu-Pro-Glu
Leu-Pro-Ile
Leu-Pro-Leu
Leu-Pro-Phe
Leu-Pro-Val
Leu-Thr-Val
Leu-Tyr-Val
Lys
Lys₂-Leu₂-Ile [SEQ ID NO: 17]
Lys₂-Pro₂-Ile [SEQ ID NO: 18]
Lys-Asp-Val
Lys-Glu-Val
Lys-Gly-Val
Lys-Ile-Val
Lys-Leu-Val
Lys-Lys
Lys-Lys-Ile
Lys-Lys-Leu
Lys-Lys-Val
Lys-Phe-Val
Lys-Pro-Val
Lys-Thr-Val
Lys-Tyr-Val
Lys-Tyr-Val-lle [SEQ ID NO: 1]
Lys-Val-Val
Met
Phe
Phe₂-Glu₂Ile [SEQ ID NO: 19]
Phe₂-Lys(Phe₂) [SEQ ID NO: 31]
Phe_{5 [SEQ ID NO: 20]}
Phe-Asp-Val
Phe-Glu-Val
Phe-Gly-Val
Phe-Ile-Val
Phe-Leu-Val
Phe-Lys-Val
Phe-Phe-Cha
Phe-Phe-hPhe
Phe-Phe-Ile
Phe-Phe-Leu
Phe-Phe-Lys-Phe-Phe
Phe-Phe-Nle
Phe-Phe-Phe
Phe-Phe-Val
Phe-Pro-Val
Phe-Ser-Val
Phe-Thr-Val
Phe-Tyr-Val
Pro
Pro₂-Lys(Pro₂) [SEQ ID NO: 32]
Pro-Asp-Val
Pro-Gly-Val
Pro-Ile-Ile
Pro-Ile-Val
Pro-Leu-Ile
Pro-Lys-Val
Pro-Phe-Ile
Pro-Phe-Val
Pro-Pro-Cha
Pro-Pro-Glu
Pro-Pro-Ile
Pro-Pro-Leu
Pro-Pro-Nle
Pro-Pro-Phe
Pro-Pro-Phe
Pro-Pro-Val
Pro-Ser-Val
Pro-Thr-Val
Pro-Tyr-Val
Pro-Val-Val
pyroglutamic acid-Glu
Ser
Ser-Asp-Val
Ser-Glu-Val
Ser-Gly-Val
Ser-Ile-Val
Ser-Leu-Val
Ser-Lys-Val
Ser-Phe-Val
Ser-Pro-Val
Ser-Tyr-Val
Ser-Val-Val
β-Leu
Thr
Thr₂-Gly₂-Ile [SEQ ID NO: 21]
Thr₂-Phe₂-Ile [SEQ ID NO: 22]
Thr-Asp-Val
Thr-Glu-Val
Thr-Gly-Val
Thr-Leu-Val
Thr-Lys-Val
Thr-Phe-Val
Thr-Pro-Val
Thr-Ser-Val
Thr-Thr-Ile
Thr-Thr-Val
Thr-Tyr-Val
Thr-Val-Val
t-Leu
Trp
Tyr
Tyr₂-Leu₂-Ile [SEQ ID NO: 23]
Tyr₂-Lys(Tyr₂) [SEQ ID NO: 33]
Tyr₂-Phe₂-Ile [SEQ ID NO: 24]
Tyr₂-Pro₂-Ile [SEQ ID NO: 25]
Tyr₂-Pro-Phe-Ile [SEQ ID NO: 26]
Tyr-Asp-Val
Tyr-Glu-Val
Tyr-Gly-Val
Tyr-Ile-Val
Tyr-Leu-Val
Tyr-Lys-Val
Tyr-Phe-Val
Tyr-Pro-Val
Tyr-Pro-Val-lle [SEQ ID NO: 2]
Tyr-Ser-Val
Tyr-Thr-Val
Tyr-Tyr-Ala
Tyr-Tyr-Cha
Tyr-Tyr-hPhe
Tyr-Tyr-Ile
Tyr-Tyr-Leu
Tyr-Tyr-Lys-Tyr-Tyr
Tyr-Tyr-Nle
Tyr-Tyr-Phe
Tyr-Tyr-Phe
Tyr-Tyr-Phe-Phe-Ile [SEQ ID NO: 27]
Tyr-Tyr-Phe-Phe-Val [SEQ ID NO: 28]
Tyr-Tyr-Val
Tyr-Val-Val
Tyr-β-Ala
Val
Val-Asp-Val
Val-Gln-Val
Val-Glu-Gly
Val-Glu-Leu
Val-Glu-Val
Val-Gly-Glu
Val-Gly-Val
Val-Phe-Val
Val-Pro-Tyr
Val-Pro-Val
Val-Thr-Val
Val-Tyr-Asp
Val-Tyr-Glu
Val-Tyr-Gly
Val-Tyr-Ile
Val-Tyr-Leu
Val-Tyr-Lys
Val-Tyr-Phe
Val-Tyr-Pro
Val-Tyr-Val
Val-Val
β-Ala
β-Ala-β-Ala

### Hydrocodone Examples

Hydrocodone may be bound to one or more chemical moieties, denominated X and Z. A chemical moiety can be any moiety that decreases the pharmacological activity of hydrocodone while bound to the chemical moiety as compared to unbound (free) hydrocodone. The attached chemical moiety can be either naturally occurring or synthetic. In one embodiment, the invention provides an hydrocodone prodrug of Formula IA or IB:

H—Xₙ-Zₘ (IA)

H-Zₘ-Xₙ (IB)

wherein H is an hydrocodone;
each X is independently a chemical moiety;
each Z is independently a chemical moiety that acts as an adjuvant and is different from at least one X;
n is an increment from 1 to 50, preferably 1 to 10; and
m is an increment from 0 to 50, preferably 0.
When m is 0, the hydrocodone prodrug is a compound of Formula (II):

H—Xₙ (II)

wherein each X is independently a chemical moiety.

Formula (II) can also be written to designate the chemical moiety that is physically attached to the hydrocodone:

H—X₁—(X)ₙ₋₁ (III)

wherein H is hydrocodone; X₁ is a chemical moiety, preferably a single amino acid; each X is independently a chemical moiety that is the same as or different from Xᵢ; and n is an increment from 1 to 50.

H is hydrocodone and has the following structure where substitution occurs at the 6 position of hydrocodone wherein A represents the attachment site for X.

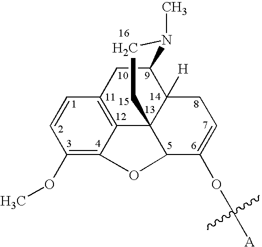

In an alternative embodiment, the 3 position and/or the N position of hydrocodone may be substituted with a chemical moiety with or without the presence of a linker. See U.S. Pat. No. 5,610,283 for methods of substituting opioids at these positions. Chemical moieties include, but are not limited to any of the carrier peptides listed below in Table 3.

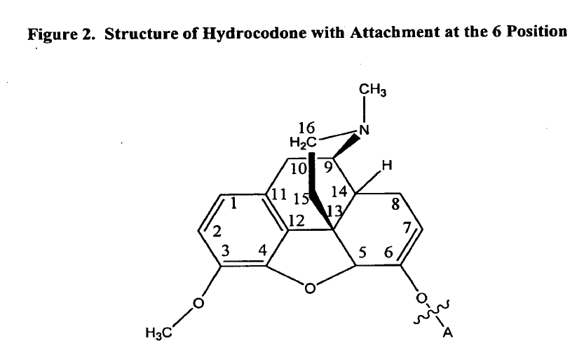

Referring now to FIG. 2, this Figure shows the potential attachment sites of hydrocodone. Specifically, hydrocodone may be attached to the chemical moiety at the 6 positions.

The following Table lists preferred hydrocodone conjugates made according to the invention.

List of Hydrocodone (HC) Conjugates attached through the 6 position to the C-terminus of the amino acid According to the invention (for clarity purposes the amino acid that is next to the —HC is the amino acid that is connected to the HC).

| | | |
|---|---|---|
| Aib-HC | Phe-Phe-Ile-HC | Lys₂-Gly₂-Ile-HC |
| Boc-Glu(OtBu)-HC | Phe-Phe-Leu-HC | Lys₂-Leu₂-Ile-HC [SEQ ID |
| | | NO: XX] |
| Boc-Lys(Boc)-HC | Phe-Phe-Phe-HC | Lys₂-Pro₂-Ile-HC [SEQ ID |
| | | NO: XX] |
| Glu-HC | Pro-Ile-Ile-HC | Phe₂-Glu₂-Ile-HC [SEQ ID |
| | | NO: XX] |
| Gly-HC | Pro-Leu-Ile-HC | Phe₅-HC [SEQ ID NO: XX] |
| Ile-HC | Pro-Phe-Ile-HC | Thr₂-Gly₂-Ile-HC [SEQ ID |
| | | NO: XX] |
| Leu-HC | Pro-Pro-Glu-HC | Thr₂-Phe₂-Ile-HC [SEQ ID |
| | | NO: XX] |
| Lys-HC | Pro-Pro-Ile-HC | Tyr₂-Glu₂-Ile-HC [SEQ ID |
| | | NO: XX] |
| Phe-HC | Pro-Pro-Leu-HC | Tyr₂-Gly₂-Ile-HC [SEQ ID |
| | | NO: XX] |
| Pro-HC | Pro-Pro-Phe-HC | Tyr₂-Leu₂-Ile-HC [SEQ ID |
| | | NO: XX] |
| Ser-HC | Thr-Thr-Ile-HC | Tyr₂-Phe-Pro-Ile-HC [SEQ ID |
| | | NO: XX] |
| Ala-Pro-HC | Tyr-Tyr-Ile-HC | Tyr₂-Pro₂-Ile-HC [SEQ ID |
| | | NO: XX] |
| Boc-Ala-Pro-HC | Gln-Gln-Ile-HC | Tyr₂-Pro-Phe-Ile-HC [SEQ ID |
| | | NO: XX] |
| Boc-Glu(OtBu)-Leu-HC | Gly-Gly-Gly-Gly-HC [SEQ ID | Tyr-Tyr-Phe-Phe-Ile-HC [SEQ |
| | NO: XX] | ID NO: XX] |
| Boc-Glu(OtBu)-Pro-HC | Acetyl-Glu₂-Pro₂-Ile-HC [SEQ ID | Glu-Glu-Phe-Phe-Phe-Ile-HC |
| | NO: XX] | |
| Glu-Glu-HC | Asp₂-Gly₂-Ile-HC [SEQ ID NO: | β-Ala-HC |
| | XX] | |
| Glu-Leu-HC | Asp₂-Leu₂-Ile-HC [SEQ ID NO: | β-Ala-β-Ala-HC |
| | XX] | |
| Glu-Pro-HC | Asp₂-Phe₂-Ile-HC [SEQ ID NO: | EpE-HC |
| | XX] | |
| Glu_{pyro}-Glu-HC | Asp₂-Pro₂-Ile-HC [SEQ ID NO: | Ethyl Carbonate-HC |
| | XX] | |
| Asp-Asp-Ile-HC | Asp₄-Ile-HC [SEQ ID NO: XX] | Galactose-CO-Leu-HC |
| Gln-Gln-Ile-HC | Glu₂-Asp₂-Ile-HC [SEQ ID NO: | Galactose-CO-Pro₂-Ile-HC |
| | XX] | |
| Glu-Glu-Glu-HC | Glu₂-Gly₂-Aib-HC | Galactose-CO-Pro₂-Leu-HC |
| Glu-Glu-Ile-HC | Glu₂-Gly₂-Ile-HC [SEQ ID NO: | galactose-Gly-Gly-Ile-HC |
| | XX] | |
| Glu-Glu-Leu-HC | Glu₂-Gly₂-Leu-HC [SEQ ID NO: | galactose-Gly-Gly-Leu-HC |
| | XX] | |
| Gly-Gly-Aib-HC | Glu₂-Gly₂-Phe-HC [SEQ ID NO: | galactose-Ile-HC |
| | XX] | |
| Gly-Gly-Glu-HC | Glu₂-Leu₃-HC [SEQ ID NO: XX] | Gulonic acid-Ile-HC |
| Gly-Gly-Ile-HC | Glu₂-Phe₂-Leu-HC [SEQ ID NO: | |
| | XX] | |
| Gly-Gly-Leu-HC | Glu₂-Phe-Pro-Ile-HC [SEQ ID NO: | |
| | XX] | |
| Gly-Gly-Phe-HC | Glu₂-Pro₂-Leu-HC [SEQ ID NO: | |
| | XX] | |
| Gly-Ile-Ile-HC | Glu₂-Pro-Phe-Ile-HC [SEQ ID NO: | |
| | XX] | |
| Gly-Leu-Ile-HC | Glu₄-Ile-HC [SEQ ID NO: XX] | |
| Gly-Leu-Leu-HC | Glu₅-HC [SEQ ID NO: XX] | |
| Gly-Phe-Ile-HC | Glu-Glu-Phe-Phe-Ile-HC [SEQ ID | |
| | NO: XX] | |
| Gly-Phe-Leu-HC | Glu-Glu-Phe-Phe-Phe-HC [SEQ ID | |
| | NO: XX] | |
| Leu-Leu-Glu-HC | Gly₂-Glu₂-Ile-HC [SEQ ID NO: | |
| | XX] | |
| Leu-Leu-Ile-HC | Gly₂-Glu₃-HC [SEQ ID NO: XX] | |
| Leu-Leu-Leu-HC | Gly₂-Pro₂-Ile-HC [SEQ ID NO: | |
| | XX] | |
| Leu-Pro-Glu-HC | Gly₄-Aib-HC [SEQ ID NO: XX] | |
| Leu-Pro-Ile-HC | Gly₄-Ile-HC [SEQ ID NO: XX] | |
| Leu-Pro-Leu-HC | Gly₄-Leu-HC [SEQ ID NO: XX] | |
| Leu-Pro-Phe-HC | Gly₄-Phe-HC [SEQ ID NO: XX] | |
| (d)-Lys-(l)-Lys-Ile-HC | Lys₂-Asp₂-Ile-HC [SEQ ID NO: | |
| | XX] | |
| Lys-Lys-Ile-HC | Lys₂-Glu₂-Ile-HC [SEQ ID NO: | |
| | XX] | |

The Examples illustrate the applicability of attaching various moieties to hydrocodone to reduce the potential for overdose while maintaining therapeutic value. The invention is illustrated by pharmacokinetic studies with various peptide opioid (e.g. hydrocodone) conjugates. The pharmacokinetics of the parent opioid (e.g. hydrocodone) and major active metabolites (e.g. hydromorphone and oxymorphone) following oral, intravenous, or intranasal administration of the peptide-opioid conjugate or the parent drug at equimolar amounts were determined in rats.

Oral, intranasal, and intravenous bioavailability studies of hydrocodone and hydrocodone conjugates were conducted in male Sprague-Dawley rats. Doses of hydrocodone bitartrate and hydrocodone conjugates containing equivalent amounts of hydrocodone were administered in deionized water. Oral administration was in 0.5 ml by gavage needle (with the exception of YYI-HC, which was delivered as a solid in gelatin capsules). Intranasal doses were administered by placing 20 microliters into the nasal flares of rats anesthetized with isoflurane. Intravenous administration was in 0.1 ml by tail vein injection. Plasma was collected by retroorbital sinus puncture under isoflurane anesthesia. Hydrocodone and hydromorphone (major active metabolite) concentrations were determined by LC/MS/MS.

The below examples are illustrative only and the below amino acid sequences attached to hydrocodone is not meant to be limiting. As such, synthesis and attachment of hydrocodone may be accomplished for instance view the following exemplary methods.

Peptide conjugates were synthesized by the general method described in below.

Hydrocodone free base was treated with a base (LHMTS, K-t-BuO, Li-t-BuO) followed by addition of N-protected activated amino acid. The product then obtained was nitrogen deprotected to yield an amino-acid linked hydrocodone.

An iterative approach can be used to identify favorable conjugates by synthesizing and testing single amino acid conjugates, and then extending the peptide one amino acid at a time to yield dipeptide and tripeptide conjugates, etc. The parent single amino acid prodrug candidate may exhibit more or less desirable characteristics than its di- or tripeptide offspring candidates.

### Mono-Substituted Hydrocodone Conjugates

### Single Amino Acid Hydrocodone Conjugates

### Example 1

### Leu-Hydrocodone

| Reagents | | MW | Weight | | mmoles | Molar Equivalents |
|---|---|---|---|---|---|---|
| 1. | Hydrocodone | 299 | 1.00 | g | 3.34 | 1.0 |
| 1. | LiN(TMS)₂ in | 1M | 10.5 | ml | 10.5 | 3.15 |
| | THF | | | | | |
| 1. | THF | — | 25 | ml | — | — |
| 2. | Boc-Leu-OSu | 328 | 3.28 | g | 10.0 | 3.0 |

To a solution of hydrocodone in THF was added LiN(TMS)₂ in THF via syringe. The solution was stirred at ambient temperatures for 5 minutes then Boc-Leu-OSu was added. The resulting reaction mixture was stirred at ambient temperatures for 18 hours. Reaction was neutralized to pH 7 with 6M HCl. Solvent was removed. Crude material was taken up in CHCl₃ (100 ml), washed with sat. NaHCO₃ (3×100 ml), dried over MgSO₄, filtered, and solvent removed. Solid was collected as a yellow powder (1.98 g, 95% yield): ¹H NMR (DMSO-d₆) δ 0.86 (dd, 6H), 1.31 (s, 9H), 1.46 (s, 2H), 1.55 (m, 2H), 1.69 (m, 1H), 1.87 (dt, 1H), 2.07 (dt, 2H), 2.29 (s, 3H), 2.43 (m, 2H), 2.93 (d, 1H), 3.11 (s, 1H), 3.72 (s, 3H), 3.88 (dt, 1H), 4.03 (dt, 1H), 4.87 (s, 1H), 5.51 (d, 1H), 6.65 (d, 1H), 6.73 (d, 1H), 6.90 (s, 1H).

To the Boc-Leu-Hydrocodone was added 25 ml of 4N HCl in dioxane. The resulting mixture was stirred at ambient temperatures for 18 hours. Solvent was removed and final product dried under vacuum. Solid was collected as a slightly yellow solid (1.96 g, 97% yield): ¹H NMR (DMSO-d₆) δ 0.94 (d, 6H), 1.52 (m, 1H), 1.75-1.90 (m, 4H), 2.22 (dt, 1H), 2.34 (dt, 1H), 2.64 (q, 1H), 2.75 (s, 3H), 2.95-3.23 (m, 4H), 3.74 (s, 3H), 3.91 (d, 1H), 4.07 (s, 1H), 5.10 (s, 1H), 5.72 (d, 1H), 6.76 (d, 1H), 6.86 (d, 1H), 8.73 br s, 3H).

### Dipeptide Hydrocodone Conjugates

### Example 2

### Example of Conjugates Containing Two Different Amino Acids: Ala-Pro-Hydrocodone

| Reagents | MW | Weight | | mmoles | Molar Equivalents |
|---|---|---|---|---|---|
| Pro-Hydrocodone | 468 | 0.25 | g | 0.53 | 1.0 |
| Boc-Ala-OSu | 286 | 0.33 | g | 1.2 | 2.26 |
| NMM | 101 | 0.50 | ml | 5.38 | 10.2 |
| DMF | — | 10 | ml | — | — |

To a solution of Pro-Hydrocodone in DMF was added NMM followed by Boc-Ala-OSu. The solution was stirred at ambient temperatures for 18 hours. Solvent was removed. Crude material was purified using preparative HPLC (Phenomenex Luna C18, 30×250 mm, 5 μM, 100 Å; Gradient: 100 water/0 0.1% TFA-MeCN→0/100; 30 ml/min.). Solid was collected as a slightly yellow powder (0.307 g, 85% yield): ¹H NMR (DMSO-d₆) δ 1.16 (d, 3H), 1.35 (s, 9H), 1.51 (m, 2H), 1.86-2.10 (m, 6H), 2.50 (m, 1H), 2.54 (m, 1H), 2.69 (m, 1H), 2.88 (s, 3H), 3.02 (dd, 1H), 3.26 (d, 1H), 3.55 (m, 1H), 3.67 (m, 1H), 3.72 (s, 3H), 3.80 (s, 1H), 4.25 (m, 1H), 4.43 (d, 1H), 5.01 (s, 1H), 5.59 (d, 1H), 6.75 (d, 1H), 6.88 (d, 1H), 6.99 (t, 1H), 9.91 (br s, 1H).

To the Boc-Ala-Pro-Hydrocodone (0.100 g) was added 10 ml of 4 N HCl in dioxane. The resulting mixture was stirred at ambient temperatures for 18 hours. Solvent was removed and final product dried under vacuum. Solid was collected as a slightly yellow solid (0.56 g, 71% yield): ¹H NMR (DMSO-d₆) δ 1.38 (s, 3H), 1.48 (t, 1H), 1.80-2.29 (m, 8H), 2.65 (m, 1H), 2.80 (s, 3H), 2.96 (m, 3H), 3.23 (m, 2H), 3.76 (s, 3H), 3.92 (s, 1H), 4.22 (s, 1H), 4.53 (s, 1H), 5.00 (s, 1H), 5.84 (d, 1H), 6.77 (d, 1H), 6.86 (d, 1H), 8.25 (br s, 3H).

### Example 3

### Example of Conjugates Containing Two Identical Amino acids Glu-Glu-Hydrocodone

Glu-Glu-Hydrocodone was prepared by a similar method to Example 2 except the amino acid starting material was Boc-Glu(OtBu)-OSu and the conjugate starting material was Glu-Hydrocodone.

### Tripeptide Hydrocodone Conjugates

### Example 4

### Example of Conjugates Containing Different Amino Acids: Gly-Gly-Leu-Hydrocodone

| Reagents | MW | Weight | | mmoles | Molar Equivalents |
|---|---|---|---|---|---|
| Leu-Hydrocodone | 484 | 2.21 | g | 4.56 | 1.0 |
| Boc-Gly-Gly-OSu | 329 | 3.00 | g | 9.12 | 2.0 |
| NMM | 101 | 5.0 | ml | 45.6 | 10 |
| DMF | — | 100 | ml | — | — |

To a solution of Leu-Hydrocodone in DMF was added NMM followed by Boc-Gly-Gly-OSu. The solution was stirred at ambient temperatures for 18 hours. Solvent was removed. Crude material was purified using preparative HPLC (Phenomenex Luna C18, 30×250 mm, 5 μM, 100 Å; Gradient: 90 water/10 0.1% TFA-MeCN→0/100; 30 ml/min.). Solid was collected as a slightly yellow powder (2.08 g, 73% yield): ¹H NMR (DMSO-d₆) δ 0.88 (dd, 6H), 1.38 (s, 9H), 1.53-1.72 (m, 5H), 1.89 (d, 1H), 2.15 (m, 1H), 2.67 (m, 2H), 2.94 (s, 3H), 3.05 (m, 2H), 3.25 (m, 2H), 3.56 (d, 3H), 3.76 (s, 6H), 3.98 (s, 1H), 4.35 (q, 1H), 5.04 (s, 1H), 5.59 (d, 1H), 6.77 (d, 1H), 6.85 (d, 1H), 7.04 (t, 1H), 8.01 (t, 1H), 8.30 (d, 1H), 9.99 (br s, 1H).

To the Boc-Gly-Gly-Leu-Hydrocodone (2.08 g) was added 50 ml of 4N HCl in dioxane. The resulting mixture was stirred at ambient temperatures for 18 hours. Solvent was removed and final product dried under vacuum. Solid was collected as a slightly yellow solid (1.72 g, 86% yield): ¹H NMR (DMSO-d₆) δ 0.89 (dd, 6H), 1.50-1.87 (m, 5H), 2.26 (m, 2H), 2.66 (m, 2H), 2.82-2.97 (m, 5H), 3.21 (m, 2H), 3.60 (m, 4H), 3.88 (m, 5H), 4.37 (m, 1H), 5.04 (s, 1H), 5.60 (s, 1H), 6.79 (d, 2H), 8.07 (br s, 3H), 8.54 (br s, 1H), 8.66 (br s, 1H), 11.29 (br s, 1H).

### Example 5

### Example of Conjugates Containing Three Identical Amino Acids: Glu-Glu-Glu-Hydrocodone

Glu-Glu-Glu-Hydrocodone was prepared by a similar method to Example 4 except the amino acid starting material was Boc-Glu(OtBu)-Glu(OtBu)-OSu and the conjugate starting material was Glu-Hydrocodone.

### Pentapeptide Hydrocodone Conjugates

### Example 6

### Example of Conjugates Containing Different Amino Acids: Gly-Gly-Gly-Gly-Leu-Hydrocodone

| Reagents | MW | Weight | | mmoles | Molar Equivalents |
|---|---|---|---|---|---|
| Gly-Gly-Leu- | 599 | 0.580 | g | 0.970 | 1.0 |
| Hydrocodone | | | | | |
| Boc-Gly-Gly-OSu | 329 | 0.638 | g | 1.94 | 2.0 |
| NMM | 101 | 1.06 | ml | 9.70 | 10 |
| DMF | — | 20 | ml | — | — |

To a solution of Gly-Gly-Leu-Hydrocodone in DMF was added NMM followed by Boc-Gly-Gly-OSu. The solution was stirred at ambient temperatures for 18 hours. Solvent was removed. Crude material was purified using preparative HPLC (Phenomenex Luna C18, 30×250 mm, 5 μM, 100 Å; Gradient: 85 water/15 0.1% TFA-MeCN→50/50; 30 ml/min.). Solid was collected as a slightly yellow powder (0.304 g, 37% yield).

To the Boc-Gly-Gly-Gly-Gly-Leu-Hydrocodone (0.304 g) was added 25 ml of 4N HCl in dioxane. The resulting mixture was stirred at ambient temperatures for 18 hours. Solvent was removed and final product dried under vacuum. Solid was collected as a slightly yellow solid (0.247 g, 97% yield): ¹H NMR (DMSO-d₆) δ 0.87 (m, 6H), 1.23 (s, 1H), 1.51-1.86 (m, 4H), 2.18 (m, 1H), 2.71 (m, 2H), 2.77 (s, 3H), 2.96 (m, 2H), 3.17 (m, 2H), 3.61 (s, 3H), 3.81-3.84 (m, 10H), 4.22 (m, 1H), 4.36 (m, 1H), 5.09 (m, 1H), 5.59 (d, 1H), 6.74 (dd, 2H), 8.16 (br s, 4H), 8.38 (br s, 1H), 8.74 (br s, 1H), 11.42 (br s, 1H).

### Example 7

### Example of Conjugates Containing Different Amino Acids Glu₂-Gly₂-Ile-Hydrocodone

Glu₂-Gly₂-Ile-Hydrocodone was prepared by a similar method to Example 6 except the amino acid starting material was Boc-Glu(OtBu)-Glu(OtBu)-OSu and the conjugate starting material was Gly₂-Ile-Hydrocodone.

### Example 8

### Example of Conjugates Containing Different Amino Acids Gly₄-Ile-Hydrocodone

Glu₄-Ile-Hydrocodone was prepared by a similar method to Example 6 except the amino acid starting material was Boc-Gly-Gly-OSu and the conjugate starting material was Gly₂-Ile-Hydrocodone.

### Example 9

### Example of Conjugates Containing Different Amino Acids Glu₂-Phe₃-Hydrocodone

Glu₂-Phe₃-Hydrocodone was prepared by a similar method to Example 6 except the amino acid starting material was Boc-Glu(OtBu)-Glu(OtBu)-OSu and the conjugate starting material was Phe₃-Hydrocodone.

### Example 10

### Example of Conjugates Containing Different Amino Acids Tyr₂-Phe-Pro-Ile-Hydrocodone

Tyre-Phe-Pro-Ile-Hydrocodone was prepared by a similar method to Example 6 except the amino acid starting material was Boc-Tyr(tBu)-Tyr(tBu)-OSu and the conjugate starting material was Phe-Pro-Ile-Hydrocodone.

### Example 11

### Example of Conjugates Containing Five Identical Amino Acids: Glu₅-Hydrocodone

Glu₅-Hydrocodone was prepared by a similar method to Example 6 except the amino acid starting material was Boc-Glu(OtBu)-Glu(OtBu)-OSu and the conjugate starting material was Glu₃-Hydrocodone.

### Glycopeptide Hydrocodone Conjugates

| Reagents | MW | Weight | | mmoles | Molar Equivalents |
|---|---|---|---|---|---|
| 1,2:3,4-di-O- | 260 | 1.00 | g | 3.85 | 1 |
| isopropylidene-D- | | | | | |
| galactopyranose | | | | | |
| 20% Phosgene | — | 20 | ml | — | — |
| in toluene | | | | | |

### Example 12

### Chloroformate of 1,2:3,4-di-O-isopropylidene-D-galactopyranose

To a stirring solution of 20% phosgene in toluene under an inert atmosphere was added 1,2:3,4-di-O-isopropylidene-D-galactopyranose via syringe. The resulting clear, colorless solution was stirred at ambient temperature for 30 minutes. After stirring, Ar(g) was bubbled through the solution for approximately 20 minutes to remove any excess phosgene. Solvent was then removed and product dried under vacuum for 18 hours. Product was used without further purification or characterization.

### Example 13

### Galactose-CO-Leu-Hydrocodone

To the chloroformate of galactose (1.5 eq) in dimethylformamide (DMF) (2 ml/mmol) was added Leu-Hydrocodone (1 eq) and 4-methylmorpholine (NMM) (6 eq). The reaction was stirred at ambient temperatures for 18 hours. Reaction was quenched by the addition of water, solvents were removed and crude product was isolated by purification with reverse-phase HPLC.

Product was deprotected using 1:1 1 M HCl:THF (1 ml/0.1 mmol) in 3 hours. Product was re-purified by reverse-phase HPLC.

### Example 14

### Galactose-CO-Pro₂-Ile-Hydrocodone

Galactose-CO-Pro₂-Ile-Hydrocodone was prepared in a manner similar to Example 13 except Pro₂-Ile-Hydrocodone was used as the conjugated starting material.

### Example 15

### Gulonic acid-Ile-Hydrocodone

Gulonic acid-Ile-Hydrocodone was prepared in a manner similar to Example 13 except Ile-Hydrocodone was used as the conjugated starting material and Gulonic acid-OSu was used as the carbohydrate starting material.

### D-amino acid Hydrocodone Conjugates

### Example 16

### (d)-Lys-(1)-Lys-Ile-Hydrocodone

To a solution of Ile-Hydrocodone in DMF was added NMM followed by Boc-(d)-Lys(Boc)-(1)-Lys(Boc)-OSu. The solution was stirred at ambient temperatures for 18 hours. Solvent was removed. Crude material was purified using preparative HPLC (Phenomenex Luna C18, 30×250 mm, 5 μM, 100 Å; Gradient: 90 water/10 0.1% TFA-MeCN→0/100; 30 ml/min.). Solid was collected as a slightly yellow powder. To the Boc-(d)-Lys(Boc)-(1)-Lys(Boc)-Hydrocodone was added 4N HCl in dioxane. The resulting mixture was stirred at ambient temperatures for 18 hours. Solvent was removed and final product dried under vacuum. Solid was collected as a slightly yellow solid.

Oral Bioavailability of Peptide-Hydrocodone Conjugates at a Dose (1 mg/kg) Approximating a Therapeutic Human Dose and at an Elevated Dose

When the peptides are conjugated to the active agent hydrocodone oral bioavailability is maintained or increased over an equivalent hydrocodone dose when the dose is administered as 1 mg/kg. This dose is the equivalent of a human dose of 10 to 14 mg for an individual weighing 70 kg (148 lbs) according to Chou et al. However, when administered orally at 5 mg/kg peak levels and bioavailability of are substantially decreased. A 5 mg/kg dose in rats approximates an 80 mg human equivalent dose (HED) of hydrocodone bitartrate; a dose that would be likely to be harmful to a nave patient in immediate release form with the potential for fatal overdose. Human equivalent doses are defined as the equivalent dose for a 60 kg person adjusted for the body surface area of the animal model. The adjustment factor for rats is 6.2. The HED for a rat dose of 5 mg/kg of hydrocodone base, for example, is equivalent to 48.39 mg (5/6.2×60) hydrocodone base; which is equivalent to 79.98 (48.39/0.605) mg hydrocodone bitartrate, when adjusted for the salt content.

Thus the peptide-hydrocodone conjugates maintain their therapeutic value at the lower dose (1 mg/kg), whereas when given at a dose above a safe level (5 mg/kg) bioavailability is decreased as compared to hydrocodone, thus diminishing the potential for overdose by oral ingestion. The decrease in bioavailability of hydrocodone from peptide hydrocodone conjugates relative to hydrocodone ranged from 9 to 70 percent.

### Example 17

### Bioavailability of Peptide-HC Conjugates by the Intranasal Route

When the peptides are conjugated to the active agent hydrocodone the bioavailability by the intravenous route is substantially decreased thereby diminishing the possibility of overdose when the drug is administered by snorting.

### Example 18

### Hydrocodone Conjugates

Bioavailability (AUC and Cmax) of various peptide-hydrocodone conjugates relative to that of hydrocodone bitartrate have been studied. At the relatively low doses of 1 and 2 mg/kg (human equivalent doses (HEDs) of 16 and 32 mg hydrocodone bitartrate) hydrocodone conjugates show comparable bioavailability to that of hydrocodone bitartrate. At the elevated doses of 5 and 25 mg/kg bioavailability of hydrocodone and hydromorphone were substantially decreased as compared to that of hydrocodone. These doses (HED of 80 and 400 mg hydrocodne bitartrate) are equivalent to amounts well above the available prescription doses of hydrocodone bitartrate which range from 2.5 to 10 mg. When delivered by the parentaral routes of intravenous and intranasal administration a substantial decrease in bioavailability of hydrocodone and hydromorphone from hydrocodone conjugates as compared to hydrocodone bitratrate was observed. These examples establish that covalent modification of an opiod (HC) via attachment of a peptide provides a method of delivering bioequivalent doses when given at doses approximating a normal prescribed dose. When administered by parenteral routes or at oral doses in excess of the intended prescription the bioavailability is substantially decreased. Collectively, the examples clearly illustrate the utility of the invention for decreasing the abuse potential of opiods.

Summary of in vivo testing of abuse resistant hydrocodone conjugates. In vivo testing of hydrocodone conjugates demonstrates for instance decreased intranasal analgesic response, decreased intravenous analgesic response, decreased subcutaneous analgesic response, decreased oral Cₘₐₓ, decreased intranasal bioavailability (AUC and Cₘₐₓ), and decreased intravenous bioavailability (AUC and Cₘₐₓ) of hydrocodone conjugates and is described in further detail below.

### Example 19

### Decreased Intranasal Analgesic Response to Hydrocodone Conjugates

Male Sprague-Dawley rats were dosed by placing 0.02 ml of water containing hydrocodone conjugate or hydrocodone bitartrate into the nasal flares. All doses contained equivalent amounts of hydrocodone base. The time (seconds) until paw lick latency was used a measure of the analgesic effect. Rats were habituated to determine baseline response. Hot plate tests were conducted at 55° C. A limit of 45 seconds was used in all testing to avoid tissue damage. All animals were humanely sacrificed following the end of testing. The paw lick latency (analgesic effect)-time curves shown in FIGS. 61 and 63 indicate the decrease in analgesia produced by the hydrocodone conjugates as compared to an equimolar (hydrocodone base) dose of hydrocodone bitartrate. The analgesic response as determined by the hot plate test is a pharmacodynamic measurement of the pharmacological effect of hydrocodone. These examples illustrate that hydrocodone conjugates decrease the analgesic effect by the intranasal route of administration as compared to hydrodone bitartrate.

### Example 20

### Decreased Intravenous Analgesic Response to Hydrocodone Conjugates

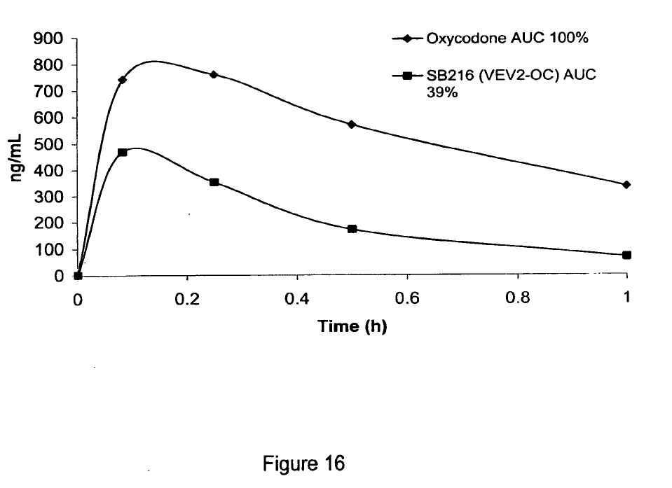

Male Sprague-Dawley rats were dosed by tail vein injection of 0.1 ml of water containing hydrocodone conjugates or hydrocodone bitartrate. All doses contained equivalent amounts of hydrocodone base. The time (seconds) until paw lick latency was used a measure of the analgesic effect. Rats were habituated to determine baseline response. Hot plate tests were conducted at 55° C. A limit of 45 seconds was used in all testing to avoid tissue damage. All animals were humanely sacrificed following the end of testing. The paw lick latency (analgesic effect)-time curve shown in FIG. 16 indicates the decrease in analgesia produced by a hydrocodone conjugate as compared to an equimolar (hydrocodone base) dose of hydrocodone bitartrate. The analgesic response as determined by the hot plate test is a pharmacodynamic measurement of the pharmacological effect of hydrocodone. This example illustrates that a hydrocodone conjugate decreased the analgesic effect by the intravenous route of administration as compared to hydrodone bitartrate.

### Example 21

### Decreased Subcutaneous Analgesic Response to Hydrocodone Conjugates

Male Sprague-Dawley rats were dosed by subcutatenous injection of 0.1 ml of water containing hydrocodone conjugates or hydrocodone bitartrate. All doses contained equivalent amounts of hydrocodone base. The time (seconds) until paw lick latency was used a measure of the analgesic effect. Rats were habituated to determine baseline response. Hot plate tests were conducted at 55° C. A limit of 45 seconds was used in all testing to avoid tissue damage. All animals were humanely sacrificed following the end of testing. The paw lick latency (analgesic effect)-time curve indicates the decrease in analgesia produced by a hydrocodone conjugate as compared to an equimolar (hydrocodone base) dose of hydrocodone bitartrate. The analgesic response as determined by the hot plate test is a pharmacodynamic measurement of the pharmacological effect of hydrocodone. This example illustrates that a hydrocodone conjugate decreased the analgesic effect by the subcutaneous route of administration as compared to hydrodone bitartrate.

### Example 22

### Decreased Oral Cₘₐₓ of Hydrocodone Conjugates

Male Sprague-Dawley rats were provided water ad libitum, fasted overnight and dosed by oral gavage with hydrocodone conjugates or hydrocodone bitartrate. All doses contained equivalent amounts of hydrocodone base. Plasma hydrocodone concentrations were measured by ELISA (Hydromorphone, 106619-1, Neogen, Corporation, Lexington, Ky.) and/or LC/MS. The assay is specific for hydromorphone (the major hydrocodone metabolite, 100% reactive) and hydrocodone (62.5% reactive). These examples illustrate that hydrocodone conjugates decrease the peak level (Cₘₐₓ) of hydrocodone plus hydromorphone as compared to that produced by equimolar (hydrocodone base) doses of hydrocodone bitartrate when given by the oral route of administration.

### Example 23

### Decreased Intranasal Bioavailability (AUC and Cₘₐₓ Hydrocodone Conjugates

Male Sprague-Dawley rats were provided water ad libitum and doses were administered by placing 0.02 ml of water containing hydrocodone conjugates or hydrocodne bitartrate into the nasal flares. All doses contained equivalent amounts of hydrocodone base. Plasma hydrocodone concentrations were measured by ELISA (Hydromorphone, 106619-1, Neogen, Corporation, Lexington, Ky.) and/or LC/MS. The assay is specific for hydromorphone (the major hydrocodone metabolite, 100% reactive) and hydrocodone (62.5% reactive). These examples illustrate that hydrocodone conjugates decrease the peak level (Cₘₐₓ) and total absorption (AUC) of hydrocodone plus hydromorphone as compared to those produced by equimolar (hydrocodone base) doses of hydrocodone bitartrate when given by the intranasal route of administration.

### Example 24

### Decreased Intravenous Bioavailability (AUC and Cₘₐₓ) Hydrocodone Conjugates

Male Sprague-Dawley rats were provided water ad libitum and doses were administered by intravenous tail vein injection of 0.1 ml of water containing hydrocodone conjugates or hydrocodone bitartrate. All doses contained equivalent amounts of d-amphetamine base. Plasma hydrocodone concentrations were measured by ELISA (Hydromorphone, 106619-1, Neogen, Corporation, Lexington, Ky.) and/or LC/MS. The assay is specific for hydromorphone (the major hydrocodone metabolite, 100% reactive) and hydrocodone (62.5% reactive). This example illustrates that a dose of hydrocodone conjugate decreases the peak level (Cₘₐₓ) and total absorption (AUC) of hydrocodone plus hydromorphone as compared to those produced by an equimolar (hydrocodone base) dose of hydrocodone bitartrate when given by the intranasal route of administration.

TABLE
Oral and Intranasal Bioavailability of Hydrocodone conjugates.

| | | stability | | | | bioavailabiltty (% HC) | | | | Oral:IN | |
|---|---|---|---|---|---|---|---|---|---|---|---|
| | | 90 C., 20 min | | | | oral | | IN | | Index | |
| Compound Class | Compound | V | BP | TW | BS | AUC | Cmax | AUC | Cmax | AUC | Cmax |
| Oral AUC >80% | | | | | | | | | | | |
| Tripeptide | Gly-Gly-Leu-HC | 1 | 100 | 85 | 100 | 82 | 126 | 77 | 62 | 1.06 | 2.03 |
| Tripeptide | Gly-Gly-Ile-HC | 0 | 100 | 93 | 100 | 95 | 167 | 93 | 103 | 1.02 | 1.62 |
| Tripeptide | Leu-Pro-Phe-HC | 2 | 100 | 100 | 100 | 106 | 125 | 83 | 101 | 1.28 | 1.24 |
| Tripeptide | Pro-Pro-Ile-HC | 0 | 16 | 70 | 100 | 112 | 99 | 59 | 65 | 1.90 | 1.52 |
| Tripeptide | Pro-Pro-Leu-HC | 2 | 100 | 100 | 100 | 94 | 108 | 46 | 48 | 2.04 | 2.25 |
| Tripeptide | Pro-Ile-Ile-HC | 0 | 47 | 83 | 100 | 104 | 99 | 86 | 102 | 1.21 | 0.97 |
| Tripeptide | Glu-Glu-Ile-HC | 0 | 94 | 26 | 100 | 83 | 112 | 52 | 59 | 1.60 | 1.90 |
| Tripeptide | Tyr-Tyr-Ile-HC | 0 | 66 | 23 | 100 | 145 | 234 | 20 | 34 | 7.25 | 6.88 |
| Tripeptide | Lys-Lys-Ile-HC | 0 | 100 | 96 | 97 | 80 | 76 | 68 | 94 | 1.18 | 0.81 |
| Tripeptide | Asp-Asp-Ile-HC | 0 | 40 | 10 | 100 | 280 | 238 | 59 | 93 | 4.75 | 2.56 |
| Tripeptide | Pro-Leu-Ile-HC | 0 | 100 | 100 | 100 | 141 | 172 | 87 | 101 | 1.62 | 1.70 |
| Tripeptide | (d)Lys(I)Lys-Ile-HC | 0 | 69 | 74 | 100 | 141 | 174 | 41 | 54 | 3.44 | 3.22 |
| Pentapeptide | Glu2-Gly2-Phe-HC | 0 | 100 | 100 | 100 | 110 | 112 | 89 | 97 | 1.24 | 1.15 |
| Pentapeptide | Glu2-Gly2-Ile-HC | 0 | 99 | 23 | 99 | 81 | 77 | 50 | 56 | 1.62 | 1.38 |
| Pentapeptide | Glu2-Phe2-Ile-HC | 0 | 100 | 33 | 100 | 96 | 129 | 68 | 76 | 1.41 | 1.70 |
| Pentapeptide | Glu2-Phe3-HC | 3 | 100 | 57 | 84 | 83 | 89 | 27 | 47 | 3.07 | 1.89 |
| Pentapeptide | Lys2-Pro2-Ile-HC | 0 | 72 | 66 | 100 | 80 | 76 | 68 | 94 | 1.18 | 0.81 |
| Pentapeptide | Tyr2-Pro2-Ile-HC | 0 | 100 | 83 | 100 | 218 | 213 | IC | IC | NA | NA |
| Pentapeptide | Asp2-Pro2-Ile-HC | 0 | 75 | 11 | 100 | 92 | 95 | 45 | 80 | 2.04 | 1.19 |
| Pentapeptide | Asp2-Gly2-Ile-HC | 0 | 68 | 3 | 100 | 82 | 80 | 48 | 67 | 1.71 | 1.19 |
| Pentapeptide | Gly2-Pro2-Ile-HC | 0 | 73 | 70 | 100 | 94 | 121 | 44 | 56 | 2.14 | 2.16 |
| Pentapeptide | Tyr2-Phe2-Ile-HC | 0 | 5 | 5 | 50 | 113 | 63 | 26 | 34 | 4.35 | 1.85 |
| Pentapeptide | Asp2-Phe2-Ile-HC | 0 | 73 | 14 | 94 | 115 | 167 | 56 | 62 | 2.05 | 2.69 |
| Pentapeptide | Glu2-Asp2-Ile-HC | 1 | 77 | 15 | 100 | 108 | 129 | 53 | 81 | 2.04 | 1.59 |
| Pentapeptide | Lys2-Asp2-Ile-HC | 0 | 64 | 0 | 100 | 90 | 121 | 39 | 56 | 2.31 | 2.16 |
| Pentapeptide | Asp4-Ile-HC | 2 | 32 | 2 | 99 | 79 | 110 | 36 | 64 | 2.19 | 1.72 |
| Pentapeptide | Gly2-Glu2-Ile-HC | 0 | 74 | 11 | 100 | 96 | 119 | 66 | 77 | 1.45 | 1.55 |
| Oral AUC >80%; IN AUC <60% | | | | | | | | | | | |
| Tripeptide | Pro-Pro-Ile-HC | 0 | 16 | 70 | 100 | 112 | 99 | 59 | 65 | 1.90 | 1.52 |
| Tripeptide | Pro-Pro-Leu-HC | 2 | 100 | 100 | 100 | 94 | 108 | 46 | 48 | 2.04 | 2.25 |
| Tripeptide | Glu-Glu-Ile-HC | 0 | 94 | 26 | 100 | 83 | 112 | 52 | 59 | 1.60 | 1.90 |
| Tripeptide | Tyr-Tyr-Ile-HC | 0 | 66 | 23 | 100 | 145 | 234 | 20 | 34 | 7.25 | 6.88 |
| Tripeptide | Asp-Asp-Ile-HC | 0 | 40 | 10 | 100 | 280 | 238 | 59 | 93 | 4.75 | 2.56 |
| Tripeptide | (d)Lys(I)Lys-Ile-HC | 0 | 69 | 74 | 100 | 141 | 174 | 41 | 54 | 3.44 | 3.22 |
| Pentapeptide | Glu2-Gly2-Ile-HC | 0 | 99 | 23 | 99 | 81 | 77 | 50 | 56 | 1.62 | 1.38 |
| Pentapeptide | Glu2-Phe3-HC | 3 | 100 | 57 | 84 | 83 | 89 | 27 | 47 | 3.07 | 1.89 |
| Pentapeptide | Tyr2-Pro2-Ile-HC | 0 | 100 | 83 | 100 | 218 | 213 | IC | IC | NA | NA |
| Pentapeptide | Asp2-Pro2-Ile-HC | 0 | 75 | 11 | 100 | 92 | 95 | 45 | 80 | 2.04 | 1.19 |
| Pentapeptide | Asp2-Gly2-Ile-HC | 0 | 68 | 3 | 100 | 82 | 80 | 48 | 67 | 1.71 | 1.19 |
| Pentapeptide | Gly2-Pro2-Ile-HC | 0 | 73 | 70 | 100 | 94 | 121 | 44 | 56 | 2.14 | 2.16 |
| Pentapeptide | Tyr2-Phe2-Ile-HC | 0 | 5 | 5 | 50 | 113 | 63 | 26 | 34 | 4.35 | 1.85 |
| Pentapeptide | Asp2-Phe2-Ile-HC | 0 | 73 | 14 | 94 | 115 | 167 | 56 | 62 | 2.05 | 2.69 |
| Pentapeptide | Glu2-Asp2-Ile-HC | 1 | 77 | 15 | 100 | 108 | 129 | 53 | 81 | 2.04 | 1.59 |
| Pentapeptide | Lys2-Asp2-Ile-HC | 0 | 64 | 0 | 100 | 90 | 121 | 39 | 56 | 2.31 | 2.16 |
| Pentapeptide | Asp4-Ile-HC | 2 | 32 | 2 | 99 | 79 | 110 | 36 | 64 | 2.19 | 1.72 |
| Tripeptide | Glu-Glu-Ile-HC | 0 | 94 | 26 | 100 | 83 | 112 | 52 | 59 | 1.60 | 1.90 |
| Tripeptide | Tyr-Tyr-Ile-HC | 0 | 66 | 23 | 100 | 145 | 234 | 20 | 34 | 7.25 | 6.88 |
| Tripeptide | Asp-Asp-Ile-HC | 0 | 40 | 10 | 100 | 280 | 238 | 59 | 93 | 4.75 | 2.56 |
| Pentapeptide | Glu2-Gly2-Ile-HC | 0 | 99 | 23 | 99 | 81 | 77 | 50 | 56 | 1.62 | 1.38 |
| Pentapeptide | Asp2-Pro2-Ile-HC | 0 | 75 | 11 | 100 | 92 | 95 | 45 | 80 | 2.04 | 1.19 |
| Pentapeptide | Asp2-Gly2-Ile-HC | 0 | 68 | 3 | 100 | 82 | 80 | 48 | 67 | 1.71 | 1.19 |
| Pentapeptide | Tyr2-Phe2-Ile-HC | 0 | 5 | 5 | 50 | 113 | 63 | 26 | 34 | 4.35 | 1.85 |
| Pentapeptide | Asp2-Phe2-Ile-HC | 0 | 73 | 14 | 94 | 115 | 167 | 56 | 62 | 2.05 | 2.69 |
| Pentapeptide | Glu2-Asp2-Ile-HC | 1 | 77 | 15 | 100 | 108 | 129 | 53 | 81 | 2.04 | 1.59 |
| Pentapeptide | Lys2-Asp2-Ile-HC | 0 | 64 | 0 | 100 | 90 | 121 | 39 | 56 | 2.31 | 2.16 |
| Pentapeptide | Asp4-Ile-HC | 2 | 32 | 2 | 99 | 79 | 110 | 36 | 64 | 2.19 | 1.72 |

Collectively, the examples illustrate the application of the invention for reducing the overdose potential of hydrocodone. These examples establish that hydrocodone can be covalently modified by attachment of a chemical moiety in a manner that maintains therapeutic value over a normal dosing range, while substantially decreasing if not eliminating the possibility of Overdose By Oral, Intranasal, Or Intravenous Routes Of Administration With The Hydrocodone.

### Hydromorphone Examples

Hydromorphone may be bound to one or more chemical moieties, denominated X and Z. A chemical moiety can be any moiety that decreases the pharmacological activity of hydromorphone while bound to the chemical moiety as compared to unbound (free) hydromorphone. The attached chemical moiety can be either naturally occurring or synthetic. In one embodiment, the invention provides an hydromorphone prodrug of Formula IA or IB:

H—Xₙ-Zₘ (IA)

H-Zₘ-Xₙ (IB)

wherein H is hydromorphone;
each X is independently a chemical moiety;
each Z is independently a chemical moiety that acts as an adjuvant and is different from at least one X;
n is an increment from 1 to 50, preferably 1 to 10; and
m is an increment from 0 to 50, preferably 0.
When m is 0, the hydromorphone prodrug is a compound of Formula (II):

H—Xₙ (II)

wherein each X is independently a chemical moiety.

Formula (II) can also be written to designate the chemical moiety that is physically attached to the hydromorphone:

H—X₁—(X)ₙ₋₁ (III)

wherein H is hydromorphone; X₁ is a chemical moiety, preferably a single amino acid; each X is independently a chemical moiety that is the same as or different from X₁; and n is an increment from 1 to 50.

H is hydromorphone and has the structure (IV), (V), or (VI) wherein A and B represent possible attachment sites for X.

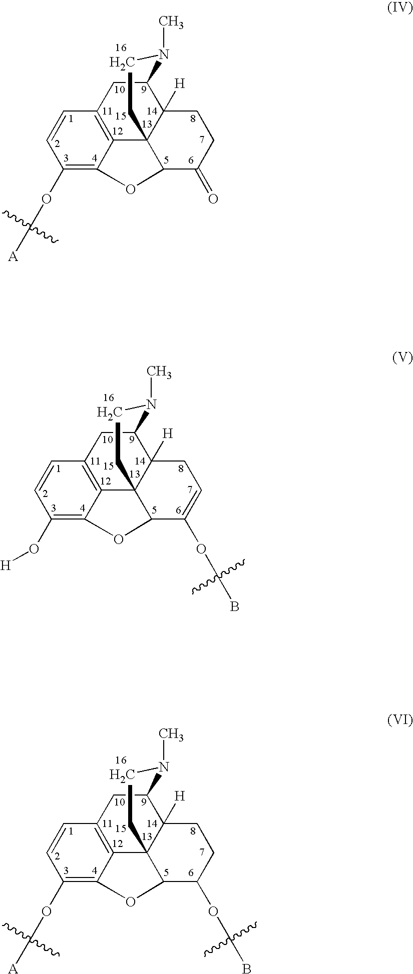

In an alternative embodiment, the N position of hydromorphone may be substituted with a chemical moiety with or without the presence of a linker. See U.S. Pat. No. 5,610,283 for methods of substituting opioids at the N position. Chemical moieties include, but are not limited to any of the carrier peptides listed below in Table 3.

The following Table lists preferred hydromorphone conjugates made according to the invention. List of Hydromorphone (BM) Conjugates attached through the 6 position to the C-terminus of the amino acid According to the invention (for clarity purposes the amino acid that is next to the —HM is the amino acid that is connected to the HM).

| | |
|---|---|
| YYFFI-HM | KKI-HM |
| EEFFF-HM | KKL-HM |
| YYI-HM | KKV-HM |
| YYL-HM | EEI-HM |
| YYV-HM | EEL-HM |
| GGI-HM | EEV-HM |
| GGL-HM | FFI-HM |
| GGV-HM | FFL-HM |
| PPI-HM | FFV-HM |
| PPL-HM | DDI-HM |
| PPV-HM | |

Hydromorphone conjugates also include the OAc and OEt derivatives of the above conjugates (at the 3 position).

### General Synthesis of Peptide Hydromorphone Conjugates

Peptide conjugates were synthesized by the general method described herein below with respect to the β-Alanine conjugate.

An iterative approach can be used to identify favorable conjugates by synthesizing and testing single amino acid conjugates, and then extending the peptide one amino acid at a time to yield dipeptide and tripeptide conjugates, etc. The parent single amino acid prodrug candidate may exhibit more or less desirable characteristics than its di- or tripeptide offspring candidates.

### General Synthesis of Single Amino Acid Hydromorphone Conjugates

Procedure for Synthesis of β-Alanine-Hydromorphone Dihydrochloride

To a stirring solution of hydromorphone free base in dimethylforamide (DMF), imidazole and then tert-butyldimethylsilyl chloride were added, under argon at ambient temperature. The solution was allowed to stir for 6 hours and the reaction was quenched with water. The solvent was removed under reduced pressure and then the crude product dissolved in ethyl acetate and washed with brine solution, dried over sodium sulfate and condensed to afford HM-TBDMS.

Hydromorphone tert-butyldimethylsilyl ether was dissolved in tetrahydrofuran (THF) under argon at ambient temperature. The solution was cooled to 0° C. and then LiN(TMS)₂ was added and the solution was allowed to stir for 10 minutes. Boc-β-Ala-OSu was then added to the solution and the reaction was monitored by HPLC. The reaction was quenched by addition of NH₄Cl solution. The solvent was removed under reduced pressure, dissolved in ethyl acetate and washed with satd. NaHCO₃ solution, brine solution and dried over sodium sulfate. The solvent was removed under reduced pressure to afford Boc-β-Ala-HM-TBDMS. The crude product was purified by preparative HPLC.

To remove the TBDMS protecting group, Boc-3-Ala-HM-TBDMS was dissolved in a 0.2 M solution of NH₄F in methanol and allowed to stir for 8 hrs, under argon at ambient temperature. The solvent was removed under reduced pressure to afford Boc-β-Ala-HM. The material was purified by crystallization in methanol and tert-butyldimethyl ether.

Boc-(3-Ala-HM was then dissolved in 4N HCl in dioxane, under argon at 0° C. and allowed to stir for 2 hours. The solvent was removed under reduced pressure to yield (3-Ala-HM

It should be recognized that synthesis for the 6 position is applicable to the 3 position as well.

The Examples are further separated into categories based on the attachment of the carrier peptides to hydromorphone. Specifically, the first category relates to Examples that are directed to mono-substituted conjugates.

### A. Mono-Substituted Hydromorphone

Within the first category, in addition to the above, Examples showing substitution at the 6 position of hydromorphone with tripeptides and pentapeptides are provided. Again, it is possible to substitute at the 3 position of hydromorphone with a chemical moiety, but this is not a preferred site of substitution.

### Attachment at the 6 Position of Hydromorphone

Example 1. Synthesis of Hydromorphone Bound to a Tripeptide at the 6 position.

To a solution of X—O⁶-hydromorphone.2HCl (1 mmol) in DMF were added NMM (10 mmol) and Boc-Z-Y—OSu (1.2 mmol). The reaction mixture was stirred at room temperature overnight. Solvent was evaporated to the residue was added saturated NaHCO₃ solution and stirred for 1 h. The precipitate was filtered, thoroughly washed with water and dried to give the title compound.

### Deprotection of Boc-Z-Y—X—O⁶-Hydromorphone:

Deprotection is performed in the same manner as the general method mentioned above to give Z-Y—X—O⁶-Hydromorphone.2HCl.

### Example 2

### Synthesis of Hydromorphone Bound to a Pentapeptide at the 6 Position

### Procedure for Synthesis of Glu₂-Phe₃-Hydromorphone.Dihydrochloride

To a stirring solution of hydromorphone free base in DMF, imidazole and then tert-butyldimethylsilyl chloride were added, under argon at ambient temperature. The solution was allowed to stir for 6 hours and then the reaction was quenched with water and the solvent was removed under reduced pressure. The crude product was dissolved in ethyl acetate and washed with brine solution, dried over sodium sulfate and condensed to afford HM-TBDMS.

Hydromorphone tert-butyldimethylsilyl ether was dissolved in THF under argon at ambient temperature. The solution was cooled to 0° C. and then LiN(TMS)₂ was added and the solution was allowed to stir for 10 minutes. Boc-Phe-OSu was then added to the solution and the reaction was monitored by HPLC. The reaction was then quenched by addition of NH₄Cl solution. The solvent was removed under reduced pressure, dissolved in ethyl acetate and washed with a satd. NaHCO₃ solution, a brine solution, dried over sodium sulfate and the solvent was removed under reduced pressure to afford Boc-Phe-HM-TBDMS. The crude product was purified by preparative HPLC.

Boc-Phe-HM-TBDMS was dissolved in 4N HCl in dioxane, under argon at 0° C. and allowed to stir for 2 hours. The solvent was removed under reduced pressure to yield Phe-HM-TBDMS.

H-Glu(OtBu)-OMe HCl was dissolved in THF and then N-methylmorpholine (NMM) and Boc-Glu(OtBu)-OSu were added and the solution was allowed to stir under argon at ambient temperature for 18 hours. The solvent was removed under reduced pressure and the product was dissolved in ethyl acetate and washed with 3% AcOH solution, satd. NaHCO₃ solution, brine solution and dried over sodium sulfate. The solvent was removed under reduced pressure to afford Boc-(Glu(OtBu))₂-OMe.

Boc-(Glu(OtBu))₂—OMe was dissolved in THF at 0° C. A solution of LiOH.H₂O in water was added and allowed to stir for 3 hours. The reaction was quenched by addition of 3% acetic acid (pH 5.5). The product was extracted in isopropyl acetate, washed with brine solution, dried over sodium sulfate and the solvent removed under reduced pressure to afford Boc-(Glu(OtBu))₂—OH.

To purify the material, Boc-(Glu(OtBu))₂—OH was dissolved in acetonitrile upon heating. Dicyclohexylamine (DCHA) was then added to the solution and allowed to cool to ambient temperature. The precipitate was filtered off and washed with acetonitrile to afford Boc-(Glu(OtBu))₂—OH.DCHA.

Boc-(Glu(OtBu))₂—OH.DCHA was dissolved in ethyl acetate and a 5% KHSO₄ solution was added and allowed to stir at ambient temperature for 20 minutes. The organic layer was separated and the product again extracted from the aqueous phase with ethyl acetate. The organic extracts were combined and the solvent removed under reduced pressure to afford Boc-(Glu(OtBu))₂—OH.

Boc-Glu(OtBu)₂-OH was dissolved in THF and the solution was cooled to 0° C., under argon. NHS was added and the solution was allowed to stir for 10 minutes. Dicyclohexycarbodiimide (DCC) was then added and the solution was allowed to warm up to ambient temperature and stirred for 18 hours. The solid (DCU) was filtered off, washed with THF and the filtrate was condensed under reduced pressure to afford Boc-(Glu(OtBu))₂—OSu.

To a solution of H-Phe₂-OMe in THF, N-methylmorpholine was added and the solution was allowed to stir for 30 minutes under argon, at 10° C. Then the solution of Boc-(Glu(OtBu))₂-OSu in THF was added and the solution was allowed to stir for 4 hours under argon, at 10° C. The reaction was quenched with a 5% NaHCO₃ solution. The product extracted with isopropyl acetate, washed with brine solution, dried over sodium sulfate and the solvent removed under reduced pressure to afford Boc-(Glu(OtBu))₂-Phe₂-OMe.

Boc-(Glu(OtBu))₂-Phe₂-OMe was dissolved in THF at 0° C. A solution of LiOH.H₂O in water was added and allowed to stir for 3 hours. The reaction was quenched by addition of 3% acetic acid (pH 5.5). The product was extracted in isopropyl acetate, washed with brine solution, dried over sodium sulfate and the solvent removed under reduced pressure to afford Boc-(Glu(OtBu))₂-Phe₂-OH.

The coupling of Boc-(Glu(OtBu))₂-Phe₂-OH and Phe-HM-TBDMS was carried out by dissolving Boc-(Glu(OtBu))₂-Phe₂-OH in THF and adding NHS and then DCC to the solution. The solution was allowed to stir at ambient temperature and stirred for 18 hours. The solid (DCU) was filtered off, washed with THF and the filtrate added to a cold solution (0° C.) of Phe-HM-TBDMS in THF and NMM. The reaction was monitored by HPLC analysis and after 5 hours the reaction was quenched by addition of a 0.5% NaHCO₃ solution. The solution was allowed to stir for 10 minutes and then water was added to the mixture. The precipitate was filtered off, washed with water and dried, yielding Boc-(Glu(OtBu))₂-Phe₃-HM-TBDMS. The product was purified by preparative HPLC.

To remove the TBDMS protecting group, Boc-(Glu(OtBu))₂-Phe₃-HM-TBDMS was dissolved in a 0.2 M solution of NH₄F in methanol, under argon at ambient temperature. Once the reaction was completed the solvent was removed under reduced pressure to afford Boc-(Glu(OtBu))₂-Phe₃-HM. The material was purified by crystallization in methanol and tert-butyldimethyl ether.

Boc-(Glu(OtBu))₂-Phe₃-HM was then dissolved in 4N HCl in dioxane, under argon at 0° C. and allowed to stir for 2 hours. The solvent was removed under reduced pressure to yield Glu₂-Phe₃-HM.

### B. Di-Substituted Hydromorphone Conjugates

The second category of Examples relates to disubstituted hydromorphone conjugates at the 3 and 6 positions. The chemical moiety may be for instance two carrier peptides varied in both length and make-up or they may be identical.

### Attachment at the 3 Position and 6 Position of Hydromorphone

### Example 3

### General Synthesis of Hydromorphone Bound to an Amino Acid at the 3 Position and at the 6 Position: [Boc-X]₂-Hydromorphone

To a solution of hydromorphone free base (2.04 g, 6.47 mmol) in THF (˜35 ml) was added LiN(TMS)₂ (19.41 ml, 19.41 mmol) and stirred for ˜30 mins. To this was added solid Boc-X—OSu (X=amino acid, 21 mmol) at one time and the reaction mixture was stirred at room temperature overnight. The solution was neutralized with 1N HCl and the THF was removed under reduced pressure. The residue was diluted with EtOAc (200 mL), satd. NaHCO₃ (150 mL) was added and stirred for 1 h. EtOAc part was washed with NaHCO₃ and brine. Dried over Na₂SO₄ and evaporated to dryness. Compound was obtained by purification over silica gel column (30% EtOAc/Hexane).

Deprotection of [Boc-X]₂-hydromorphone:

General method of deprotection: The above compound was reacted with 4N HCl/dioxane (25 mL/gm) at room temperature for 4 h. Solvent was evaporated and dried over vacuum to give X₂-Hydromorphone.3HCl.

### Pharmacokinetic Data for Hydromorphone Conjugates

The invention is illustrated by pharmacokinetic studies with hydromorphone that has been covalently modified by attachment to various moieties such as specific short chained amino acid sequences including tri-, and pentapeptides. Studies include pharmacokinetic evaluations of the various drug conjugates administered by the oral and intranasal routes. Collectively the compounds demonstrate that active agents may be modified by covalent attachment to various moieties and retain their therapeutic value at normal doses while preventing potential overdose by oral administration and prevention of abuse through intranasal administration.

The Examples illustrate the applicability of attaching various moieties to hydromorphone to reduce the potential for overdose while maintaining therapeutic value. The invention is illustrated by pharmacokinetic studies with various peptide hydromorphone conjugates. The Examples illustrate the compounds and compositions for reducing the potential for overdose and abuse while maintaining therapeutic value wherein the active agent hydromorphone (HM) is covalently attached to a chemical moiety.

Oral, and intranasal bioavailability studies of hydromorphone and hydromorphone conjugates were conducted in male Sprague-Dawley rats. Doses of hydromorphone hydrochloride and hydromorphone conjugates containing equivalent amounts of hydromorphone were administered in deionized water. Oral administration was in 0.5 ml by gavage needle. Intranasal doses were administered by placing 20 microliters into the nasal flares of rats anesthetized with isoflurane. Plasma was collected by retroorbital sinus puncture under isoflurane anesthesia, hydromorphone were determined by LC/MS/MS.

### Example 4

### Decreased oral Cₘₐₓ of Hydromorphone Conjugates

Male Sprague-Dawley rats were provided water ad libitum, fasted overnight and dosed by oral gavage with oxycodone conjugates or oxycodone HCl. All doses contained equivalent amounts of hydromorphone base. Plasma hydromorphone concentrations were measured by ELISA (102919, Neogen, Corporation, Lexington, Ky.) and/or LC/MS. These examples illustrate that doses of hydromorphone conjugates decrease the peak level (Cₘₐₓ) of hydromorphone as compared to that produced by equimolar (hydromorphone base) doses of hydromorphone HCl when given by the oral route of administration.

### Example 5

### Decreased Intranasal Bioavailability (AUC and Cₘₐₓ) of Hydromorphone Conjugates

Male Sprague-Dawley rats were provided water ad libitum and doses were administered by placing 0.02 ml of water containing hydromorphone conjugates or hydromorphone bitartrate into the nasal flares. All doses contained equivalent amounts of hydromorphone base. Plasma hydromorphone concentrations were measured by ELISA (hydromorphone, 102919, Neogen, Corporation, Lexington, Ky.) and/or LC/MS. The assay is specific for hydromorphone. These examples illustrate that hydromorphone conjugates decrease the peak level (Cₘₐₓ) and total absorption (AUC) of hydromorphone as compared to those produced by equimolar (hydromorphone base) doses of hydromorphone HCl when given by the intranasal route of administration.

The following Tables include Hydromorphone amino acid and peptide HCl salt conjugates that were synthesized and tested. Both oral and intranasal bioavailabilities were measured. The bold values are the “corrected values,” that were calculated based on the bioavailability of the control (HM).

TABLE A
Oral Bioavailability of Hydromorphone Conjugates

| | | (Y2F2I- | | | | | | | | | | (Y2F2I- |
|---|---|---|---|---|---|---|---|---|---|---|---|---|
| Oral | (Y2F2I) | HM-OAc) | (D2I) | (Y2I) | (E2F3) | (G2L) | (E2L) | (G2V) | (K2V) | (P2V) | (Y2V) | HM-OEt) |
| AUC | 74 | 54 | 46 | 52 | 128 | 41 | 51 | 86 | 78 | 40 | 53 | 149 |
| % | 40 | 29 | 25 | 32 | 79 | 22 | 27 | 53 | 43 | 24 | 30 | 71 |
| Cmax | 39 | 30 | 36 | 43 | 60 | 34 | 42 | 47 | 54 | 40 | 47 | 65 |
| % | 52 | 40 | 49 | 66 | 78 | 43 | 49 | 63 | 60 | 55 | 53 | 77 |

TABLE B
Oral Bioavailability of Hydromorphone Tripeptide Conjugates

| | | | | | | (Y2I- | | | | | | |
|---|---|---|---|---|---|---|---|---|---|---|---|---|
| Oral | (E2I) | (F2I) | (G2I) | (K2I) | (P2I) | HM-OEt) | (E2V) | (F2V) | (P2L) | (Y2L) | (F2L) | (K2L) |
| AUC | 94 | 34 | 30 | 39 | 35 | 24 | 36 | 13 | 39 | 21 | 18 | 44 |
| % | 56 | 18 | 17 | 24 | 21 | 14 | 21 | 8 | 22 | 12 | 11 | 24 |
| Cmax | 56 | 42 | 24 | 44 | 43 | 31 | 39 | 28 | 35 | 23 | 17 | 41 |
| % | 73 | 47 | 32 | 56 | 60 | 42 | 50 | 36 | 45 | 30 | 24 | 50 |

TABLE C
Intranasal Bioavailability of Hydromorphone Conjugates

| | (Y2F2I- | (Y2F2I- | | | | | | | | | | |
|---|---|---|---|---|---|---|---|---|---|---|---|---|
| IN | HM-OAc) | HM-OEt) | (Y2F2I) | (D2I) | (Y2I) | (P2L) | (Y2L) | (G2I) | (K2I) | (E2F3) | (G2L) | (E2L) |
| AUC | 51 | 48 | 28 | 387 | 619 | 352 | 491 | 147 | 421 | 384 | 357 | 575 |
| % | 59 | 56 | 4 | 55 | 88 | 53 | 74 | 24 | 68 | 52 | 52 | 84 |
| Cmax | 66 | 63 | 62 | 721 | 732 | 600 | 642 | 686 | 881 | 566 | 768 | 880 |
| % | 72 | 68 | 7 | 85 | 86 | 70 | 75 | 66 | 85 | 57 | 85 | 98 |

TABLE D
Intranasal Bioavailability of Hydromorphone Tripeptide Conjugates

| | | | | (Y2I- | | | | | | | | |
|---|---|---|---|---|---|---|---|---|---|---|---|---|
| IN | (E2I) | (F2I) | (P2I) | HM-OEt) | (E2V) | (G2V) | (K2V) | (P2V) | (Y2V) | (F2L) | (K2L) | (F2V) |
| AUC | 517 | 695 | 535 | 717 | 211 | 385 | 560 | 479 | 492 | 679 | 648 | 658 |
| % | 59 | 79 | 77 | 103 | 31 | 51 | 75 | 71 | 73 | 92 | 87 | 98 |
| Cmax | 875 | 916 | 888 | 845 | 629 | 760 | 931 | 938 | 686 | 945 | 949 | 920 |
| % | 88 | 92 | 95 | 90 | 70 | 73 | 89 | 101 | 73 | 96 | 96 | 103 |

TABLE E
Bioavailability of Hydromorphone Conjugates.

| | | | | theor. |
|---|---|---|---|---|
| Compound Class | Compound | oral AUC | IN AUC | potency |
| pentapeptide | EEFFF-HM | 79 | 52 | 27 |
| tripeptide | DDI-HM | 25 | 55 | 40 |
| tripeptide | EEI-HM | 56 | 59 | 39 |
| tripeptide | FFI-HM | 18 | 79 | 37 |
| tripeptide | GGI-HM | 17 | 24 | 48 |
| tripeptide | KKI-HM | 24 | 68 | 35 |
| tripeptide | PPI-KM | 21 | 77 | 43 |
| tripeptide | YYI-HM | 32 | 88 | 28 |
| tripeptide | YYI-HM-OAc | 29 | 73 | 34 |
| tripeptide | YYI-HM-OEt | 14 | 103 | 33 |
| pentapeptide | YYFFI-HM | 40 | 4 | 26 |
| pentapeptide | YYFFI-HM-OAc | nd | 59 | 25 |
| pentapeptide | YYFFI-HM-OEt | 71 | 56 | 24 |
| tripeptide | EEL-HM | 27 | 84 | 39 |
| tripeptide | FFL-HM | 11 | 92 | 37 |
| tripeptide | GGL-HM | 22 | 52 | 49 |
| tripeptide | KKL-HM | 24 | 87 | 35 |
| tripeptide | PPL-HM | 22 | 53 | 43 |
| tripeptide | YYL-HM | 12 | 74 | 36 |
| tripeptide | EEV-HM | 21 | 31 | 40 |
| tripeptide | FFV-HM | 8 | 98 | 38 |
| tripeptide | GGV-HM | 53 | 51 | 50 |
| tripeptide | KKV-HM | 43 | 75 | 36 |
| tripeptide | PPV-HM | 24 | 71 | 44 |
| tripeptide | YYV-HM | 30 | 73 | 36 |

Collectively, the examples illustrate the application of the invention for reducing the overdose potential of hydromorphone. These examples establish that hydromorphone can be covalently modified by attachment of a chemical moiety in a manner that maintains therapeutic value over a normal dosing range, while substantially decreasing if not eliminating the possibility of overdose by oral or intranasal routes of administration with the hydromorphone.

### Oxycodone Examples

Oxycodone may be bound to one or more chemical moieties, denominated X and Z. A chemical moiety can be any moiety that decreases the pharmacological activity of oxycodone while bound to the chemical moiety as compared to unbound (free) oxycodone. The attached chemical moiety can be either naturally occurring or synthetic. In one embodiment, the invention provides an oxycodone prodrug of Formula IA or IB:

O—Xₙ-Zₘ (IA)

O-Zₘ-Xₙ (IB)

wherein O is oxycodone;
each X is independently a chemical moiety;
each Z is independently a chemical moiety that acts as an adjuvant and is different from at least one X;
n is an increment from 1 to 50, preferably 1 to 10; and
m is an increment from 0 to 50, preferably 0.
When m is 0, the oxycodone prodrug is a compound of Formula (II):

O—Xₙ (II)

wherein each X is independently a chemical moiety.

Formula (II) can also be written to designate the chemical moiety that is physically attached to the oxycodone:

O—X₁—(X)ₙ₋₁ (III)

wherein O is oxycodone; X₁ is a chemical moiety, preferably a single amino acid; each X is independently a chemical moiety that is the same as or different from X₁; and n is an increment from 1 to 50.

O is oxycodone and upon substitution with X, may have the following structures IV, V, or VI, wherein A and B represent possible attachment sites for X.

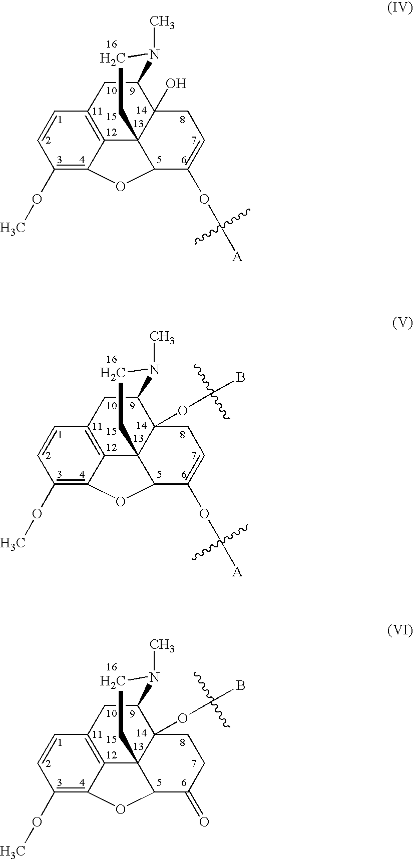

In an alternative embodiment, the 3 position and/or N position of oxycodone may be substituted with a chemical moiety with or without the presence of a linker. See U.S. Pat. No. 5,610,283 for methods of substituting opioids at these positions.

It is noted that for disubstituted conjugates, each of the sequences listed above may be present along with any other sequence to form a disubstituted oxycodone conjugate. In addition, a disubstituted oxycodone conjugate may be formed from substitution at two positions with two occurrences of any one of the sequences.

The following Table lists preferred oxycodone conjugates made according to the invention. The designation [peptide]₂—OC refers to a disubstituted oxycodone conjugate according to Structure (V) set forth above. In addition, the designation [peptide]-OC-[peptide] refers to a disubstituted oxycodone conjugate, wherein the peptide that precedes OC is bound to the 6 position of oxycodone and the peptide the follows OC is at the 14 position.

List of Oxycodone (OC) Conjugates attached through the 6 position (and also through the 14 position for disubstituted OC conjugates) to the C-terminus of the amino acid according to the invention (for clarity purposes the amino acid that is next to the —OC is the amino acid that is connected to the OC).

| | | |
|---|---|---|
| β-alanine-OC | [Asp-Lys-Val]₂-OC | [Pro-Asp-Val]₂-OC |
| Glu-OC | [Asp-Phe-Val]₂-OC | [Pro-Val-Val]₂-OC |
| Ile-OC | [Asp-Pro-Val]₂-OC | [Ser-Thr-Val]₂-OC |
| Leu-OC | [Asp-Ser-Val]₂-OC | [Ser-Asp-Val]₂-OC |
| Phe-OC | [Asp-Thr-Val]₂-OC | [Ser-Glu-Val]₂-OC |
| β-Leu-OC | [Asp-Tyr-Val]₂-OC | [Ser-Gly-Val]₂-OC |
| Val-OC | [Asp-Val-Val]₂-OC | [Ser-Ile-Val]₂-OC |
| β-Ala-β-Ala-OC | [Bio-Gly₂-Ile]₂-OC | [Ser-Leu-Val]₂-OC |
| Tyr-β-Ala-OC | [Bio-Gly₂-Leu]₂-OC | [Ser-Lys-Val]₂-OC |
| Asp-Asp-Ile-OC | [Gal-Gly₂-Ile]₂-OC | [Ser-Phe-Val]₂-OC |
| Asp-Asp-Val-OC | [Gal-Gly₂-Leu]₂-OC | [Ser-Pro-Val]₂-OC |
| Ala-Ala-Val-OC | [Gal-Pro₂-Ile]₂-OC | [Ser-Tyr-Val]₂-OC |
| Gln-Gln-β-Ala-OC | [Gal-Pro₂-Leu]₂-OC | [Ser-Val-Val]₂-OC |
| Gln-Gln-Ile-OC | [Gln-Gln-Val]₂-OC | |
| Glu-Leu-Val-OC | [Gln-Pro-Val]₂-OC | |
| Glu-Tyr-Val-OC | [Glu-Asp-Val]₂-OC | [Thr-Thr-Val]₂-OC |
| Glu-Glu-Ala-OC | [Glu-Glu-Cha]₂-OC | [Thr-Asp-Val]₂-OC |
| Glu-Glu-Ile-OC | [Glu-Glu-hPhe]₂-OC | [Thr-Glu-Val]₂-OC |
| Glu-Glu-Leu-OC | [Glu-Glu-Nle]₂-OC | [Thr-Gly-Val]₂-OC |
| Glu-Glu-Phe-OC | [Glu-Glu-Phe]₂-OC | [Thr-Leu-Val]₂-OC |
| Glu-Glu-Pro-OC | [Glu-Gly-Val]₂-OC | [Thr-Lys-Val]₂-OC |
| Glu-Glu-β-Ala-OC | [Glu-Leu-Val]₂-OC | [Thr-Phe-Val]₂-OC |
| Glu-Glu-Val-OC | [Glu-Lys-Val]₂-OC | [Thr-Pro-Val]₂-OC |
| Glu-Tyr-Val-OC-OAc | [Glu-Phe-Val]₂-OC | [Thr-Ser-Val]₂-OC |
| Glu-Tyr-Val-OC-OCOOEt | [Glu-Pro-Val]₂-OC | [Thr-Tyr-Val]₂-OC |
| Gly-Gly-Ile-OC | [Glu-Ser-Val]₂-OC | [Thr-Val-Val]₂-OC |
| Gly-Gly-Leu-OC | [Glu-Thr-val]₂-OC | [Tyr-Pro-Val]₂-OC |
| Gly-Gly-Phe-OC | [Glu-Tyr-Val]₂-OC | [Tyr-Tyr-Cha]₂-OC |
| Gly-Gly-β-Ala-OC | [Glu-Val-Val]₂-OC | [Tyr-Tyr-hPhe]₂-OC |
| Gly-Gly-Val-OC | [Gly-Gly-Cha]₂-OC | [Tyr-Tyr-Nle]₂-OC |
| Ile-Ile-Ile-OC | [Gly-Gly-hPhe]₂-OC | [Tyr-Tyr-Phe]₂-OC |
| Ile-Tyr-Val-OC | [Gly-Gly-Nle]₂-OC | [Tyr-Asp-Val]₂-OC |
| Ile-Tyr-Val-OC-OAc | [Gly-Gly-Phe]₂-OC | [Tyr-Glu-Val]₂-OC |
| Ile-Tyr-Val-OC-OCOOEt | [Gly-Gly-Val]₂-OC | [Tyr-Gly-Val]₂-OC |
| Leu-Leu-Ala-OC | [Gly-Leu-Val]₂-OC | [Tyr-Ile-Val]₂-OC |
| Leu-Leu-Ile-OC | [Gly-Asp-Val]₂-OC | [Tyr-Leu-Val]₂-OC |
| Leu-Leu-Leu-OC | [Gly-Glu-Val]₂-OC | [Tyr-Lys-Val]₂-OC |
| Leu-Leu-Val-OC | [Gly-Lys-Val]₂-OC | [Tyr-Phe-Val]₂-OC |
| Leu-Leu-β-Ala-OC | [Gly-Phe-Val]₂-OC | [Tyr-Ser-Val]₂-OC |
| Leu-Tyr-Val-OC | [Gly-Pro-Val]₂-OC | [Tyr-Thr-Val]₂-OC |
| Lys-Lys-Ala-OC | [Gly-Ser-Val]₂-OC | [Tyr-Tyr-Val]₂-OC |
| Lys-Lys-Ile-OC | [Gly-Thr-Val]₂-OC | [Tyr-Val-Val]₂-OC |
| Lys-Lys-Leu-OC | [Gly-Tyr-Val]₂-OC | [Val-Glu-Val]₂-OC |
| | | [Val-Gln-Val]₂-OC |
| Lys-Lys-Phe-OC | [Gly-Val-Val]₂-OC | [Val-Asp-Val]₂-OC |
| Lys-Lys-Val-OC | [Ile-Tyr-Val]₂-OC | [Val-Glu-Val]₂-OC |
| Lys-Lys-β-Ala-OC | [Ile-Asp-Val]₂-OC | [Val-Gly-Val]₂-OC |
| Lys-Tyr-Val-OC-OAc | [Ile-Glu-Val]₂-OC | [Val-Phe-Val]₂-OC |
| Lys-Tyr-Val-OC-OCOOEt | [Ile-Gly-Val]₂-OC | [Val-Pro-Val]₂-OC |
| Phe-Phe-Leu-OC | [Ile-Phe-Val]₂-OC | [Val-Thr-Val]₂-OC |
| Phe-Phe-Ile-OC | [Ile-Ser-Val]₂-OC | [Val-Tyr-Val]₂-OC |
| Phe-Phe-Val-OC | [Ile-Thr-Val]₂-OC | Ile-Tyr-Val-OC-Val-Glu-Val |
| Phe-Tyr-Val-OC | [Leu-Gly-Val]₂-OC | Ile-Tyr-Val-OC-Val-Gly-Glu |
| Pro₂-Ile-OC | [Leu-Lys-Val]₂-OC | Ile-Tyr-Val-OC-Val-Pro-Tyr |
| Pro₂-Leu-OC | [Leu-Phe-Val]₂-OC | Ile-Tyr-Val-OC-Val-Tyr-Asp |
| Pro-Glu-Val-OC | [Leu-Pro-Val]₂-OC | Ile-Tyr-Val-OC-Val-Tyr-Glu |
| Pro-Pro-Ala-OC | [Leu-Thr-Val]₂-OC | Ile-Tyr-Val-OC-Val-Tyr-Gly |
| Pro-Pro-Ile-OC | [Leu-Tyr-Val]₂-OC | Ile-Tyr-Val-OC-Val-Tyr-Lys |
| Pro-Pro-Leu-OC | [Lys-Lys-Val]₂-OC | Ile-Tyr-Val-OC-Val-Tyr-Pro |
| Pro-Pro-Val-OC | [Lys-Ser-Val]₂-OC | Leu-Tyr-Val-OC-Gly-Tyr-Leu |
| Pro-Tyr-Val-OC | [Lys-Asp-Val]₂-OC | Leu-Tyr-Val-OC-Val-Glu-Gly |
| Ser-Ser-Ser-OC | [Lys-Glu-Val]₂-OC | Ile-Tyr-Val-OC-Val-Glu-Leu |
| Thr-Thr-Thr-OC | [Lys-Gly-Val]₂-OC | Ile-Tyr-Val-OC-Gly-Tyr-Ile |
| Thr-Thr-Val-OC | [Lys-Ile-Val]₂-OC | Ile-Tyr-Val-OC-Val-Glu-Gly |
| Succcinate-OC | [Lys-Leu-Val]₂-OC | Leu-Tyr-Val-OC-Val-Glu-Leu |
| Tyr-Tyr-Ala-OC | [Lys-Phe-Val]₂-OC | Leu-Tyr-Val-OC-Val -Pro-Tyr |
| Tyr-Tyr-Ile-OC | [Lys-Pro-Val]₂-OC | Leu-Tyr-Val-OC-Val-Tyr-Gly |
| Tyr-Tyr-Leu-OC | [Lys-Thr-Val]₂-OC | Lys-Tyr-Val-OC-Val-Glu-Val |
| Tyr-Tyr-Phe-OC | [Lys-Tyr-Val]₂-OC | Lys-Tyr-Val-OC-Val-Gly-Glu |
| Tyr-Tyr-Pro-OC | [Lys-Val-Val]₂-OC | Lys-Tyr-Val-OC-Val-Tyr-Asp |
| Tyr-Tyr-β-Ala-OC | [Nia-Gly₂-Ile]₂-OC | Lys-Tyr-Val-OC-Val-Tyr-Glu |
| Tyr-Tyr-Val-OC | [Nia-Gly₂-Ile]₂-OC | Lys-Tyr-Val-OC-Val-Tyr-Ile |
| Val-Val-Leu-OC | [Nia-Gly₂-Leu]₂-OC | Lys-Tyr-Val-OC-Val-Tyr-Leu |
| Val-Val-Phe-OC | [Nia-Gly₂-Leu]₂-OC | Lys-Tyr-Val-OC-Val-Tyr-Lys |
| Val-Val-Val-OC | [Phe-Phe-Cha]₂-OC | Lys-Tyr-Val-OC-Val-Tyr-Phe |
| Lys-Tyr-Val-Ile-OC [SEQ ID | [Phe-Phe-hPhe]₂-OC | Lys-Tyr-Val-OC-Val-Tyr-Pro |
| NO: X] | | |
| Tyr-Pro-Val-Ile-OC [SEQ ID | [Phe-Phe-Nle]₂-OC | Lys-Tyr-Val-OC-Val-Tyr-Val |
| NO: X] | | |
| Glu-Glu-Phe-Phe-Ile-OC | [Phe-Phe-Phe]₂-OC | Phe-Tyr-Val-OC-Val-Glu-Gly |
| [SEQ ID NO: X] | | |
| Glu-Glu-Phe-Phe-Phe-OC | [Phe-Phe-Val]₂-OC | Phe-Tyr-Val-OC-Val-Gly-Glu |
| [SEQ ID NO: X] | | |
| Phe-Phe-Lys-Phe-Phe-OC | [Phe-Val-Val]₂-OC | Phe-Tyr-Val-OC-Val-Tyr-Asp |
| [SEQ ID NO: X] | | |
| Tyr-Tyr-Lys-Tyr-Tyr-OC | [Phe-Asp-Val]₂-OC | Phe-Tyr-Val-OC-Val-Tyr-Glu |
| [SEQ ID NO: X] | | |
| Tyr-Tyr-Phe-Phe-Ile-OC [SEQ | [Phe-Glu-Val]₂-OC | Pro-Tyr-Val-OC-Val-Tyr-Glu |
| ID NO: X] | | |
| Tyr-Tyr-Phe-Phe-Val-OC | [Phe-Gly-Val]₂-OC | Pro-Tyr-Val-OC-Val-Tyr-Ile |
| [SEQ ID NO: X] | | |
| [Boc-Cha]₂-OC | [Phe-Ile-Val]₂-OC | Pro-Tyr-Val-OC-Val-Tyr-Leu |
| [Boc-Dpg]₂-OC | [Phe-Leu-Val]₂-OC | Tyr-Pro-Val-OC-Val-Tyr-Glu |
| [Boc-hPhe]₂-OC | [Phe-Lys-Val]₂-OC | Tyr-Pro-Val-OC-Val-Tyr-Ile |
| [Boc-Nle]₂-OC | [Phe-Pro-Val]₂-OC | Tyr-Pro-Val-OC-Val-Tyr-Leu |
| [Boc-Tle]₂-OC | [Phe-Ser-Val]₂-OC | [Lys-Lys-Gly-Gly]₂-OC [SEQ ID |
| | | NO: X] |
| [Boc-Val]₂-OC | [Phe-Thr-Val]₂-OC | [Asp₂-Lys(Asp₂)]₂-OC [SEQ ID |
| | | NO: X] |
| [Glu]₂-OC | [Phe-Tyr-Val]₂-OC | [Glu₂-Lys(Glu₂)]₂-OC [SEQ ID |
| | | NO: X] |
| [Ile]₂-OC | [Pro-Pro-Cha]₂-OC | [Gly₂-Lys(-Gly₂)]₂-OC [SEQ ID |
| | | NO: X] |
| [Leu]₂-OC | [Pro-Pro-Ile]₂-OC | [Phe₂-Lys(Phe₂)]₂-OC [SEQ ID |
| | | NO: X] |
| [Lys]₂-OC | [Pro-Pro-Nle]₂-OC | [Pro₂-Lys(Pro₂)]₂-OC [SEQ ID |
| | | NO: X] |
| [Phe]₂-OC | [Pro-Pro-Phe]₂-OC | [Tyr₂-Lys(Tyr₂)]₂-OC [SEQ ID |
| | | NO: X] |
| [β-Ala]₂-OC | [Leu-Asp-Val]₂-OC | |
| [Val]₂-OC | [Leu-Glu-Val]₂-OC | |
| Val-OC-Gly | [Pro-Pro-Leu]₂-OC | |
| [Asp-Asp-Cha]₂-OC | [Pro-Glu-Val]₂-OC | |
| [Asp-Asp-Nle]₂-OC | [Pro-Gly-Val]₂-OC | |
| [Asp-Asp-Phe]₂-OC | [Pro-Ile-Val]₂-OC | |
| [Asp-Asp-Val]₂-OC | [Pro-Lys-Val]₂-OC | |
| [Asp-d-Asp-Ile]₂-OC | [Pro-Phe-Val]₂-OC | |
| [Asp-Glu-Val]₂-OC | [Pro-Ser-Val]₂-OC | |
| [Asp-Gly-Val]₂-OC | [Pro-Thr-Val]₂-OC | |
| [Asp-Ile-Val]₂-OC | [Pro-Tyr-Val]₂-OC | |
| [Asp-Leu-Val]₂-OC | [Pro-Pro-Val]₂-OC | |

Oxycodone conjugates also include the OAc and OEt derivatives of the above conjugates (in the case of mono-conjugates).

Peptide conjugates were synthesized by the general method described below.

### General Structure of Oxycodone Derivatives:

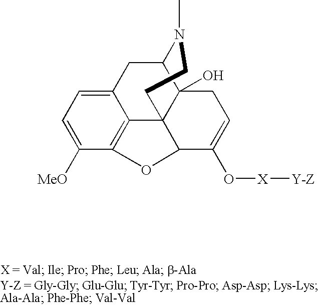

### Synthetic Scheme of Oxycodone Derivatives:

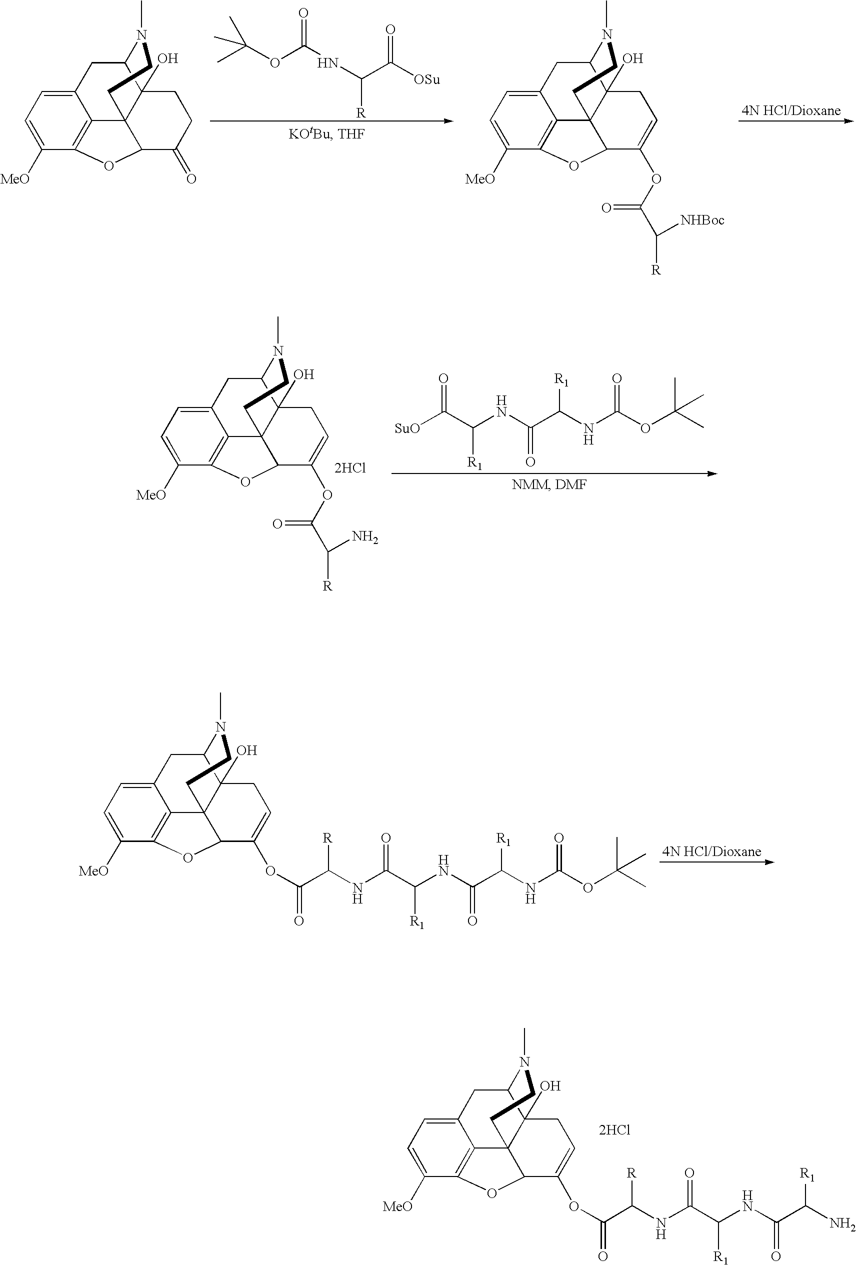

The above general synthesis scheme was applied to give the following preferred sequences of amino acids with oxycodone and bioavailability as set forth in Table 3. Exemplary bioavailability of oxycodone compounds.

| | | Oral |
|---|---|---|
| ENTRY | OXYCODONE COMPOUND | (% AUC) |
| | OXY | 100 |
| 1 | Gly-Gly-Val-OC | 123 |
| 2 | Ala-Ala-Val-OC | 85 |
| 3 | Glu-Glu-Val-OC | 55 |
| 4 | Lys-Lys-Val-OC | 108 |
| 5 | Leu-Leu-Val-OC | 81 |
| 6 | Tyr-Tyr-Val-OC | 124 |
| 7 | Pro-Pro-Val-OC | 152 |
| 8 | Phe-Phe-Val-OC | 32 |
| 9 | Asp-Asp-Val-OC | 40 |
| 10 | Val-Val-Val-OC | |
| *11 | Gly-Gly-Ile-OC | 224 |
| 12 | Glu-Glu-Ile-OC | 179 |
| 13 | Lys-Lys-Ile-OC | 74 |
| 14 | Tyr-Tyr-Ile-OC | 85 |
| 15 | Ile-Ile-Ile-OC | 83 |
| 16 | Pro-Pro-Ile-OC | 85 |
| 17 | Phe-Phe-Ile-OC | 59 |
| 18 | Glu-Glu-Pro-OC | 71 |
| 19 | Tyr-Tyr-Pro-OC | 59 |
| 20 | Gly-Gly-Phe-OC | 163 |
| 21 | Glu-Glu-Phe-OC | 49 |
| 22 | Lys-Lys-Phe-OC | 37 |
| 23 | Val-Val-Phe-OC | 120 |
| 24 | Tyr-Tyr-Phe-OC | 73 |
| 25 | Gly-Gly-Leu-OC | |
| 26 | Glu-Glu-Leu-OC | 80 |
| 27 | Val-Val-Leu-OC | |
| 28 | Lys-Lys-Leu-OC | 46 |
| 29 | Leu-Leu-Leu-OC | |
| 30 | Tyr-Tyr-Leu-OC | |
| 31 | Pro-Pro-Leu-OC | |
| 32 | Phe-Phe-Leu-OC | |
| 33 | Glu-Glu-Ala-OC | |
| 34 | Leu-Leu-Ala-OC | |
| 35 | Lys-Lys-Ala-OC | |
| 36 | Pro-Pro-Ala-OC | |
| 37 | Tyr-Tyr-Ala-OC | |
| 38 | Gly-Gly-β-Ala-OC | |
| 39 | Glu-Glu-β-Ala-OC | |
| 40 | Leu-Leu-β-Ala-OC | |
| 41 | Lys-Lys-β-Ala-OC | |
| 42 | Tyr-Tyr-β-Ala-OC | |
| 43 | OC-Succcinate | 79 |
| 44 | OC-β-alanine | 144 |

*Dosed at 40% higher levels than calculated
each experiment conducted on n = 4 animals
analysis by LC-MS

An iterative approach can be used to identify favorable conjugates by synthesizing and testing single amino acid conjugates, and then extending the peptide one amino acid at a time or through the attachment of peptides to yield dipeptide and tripeptide conjugates, etc. The parent single amino acid prodrug candidate may exhibit more or less desirable characteristics than its di- or tripeptide offspring candidates.

### I. Mono-Substituted Oxycodone Conjugates

### Single Amino Acids

### Example 1

### Phe-Oxycodone-Substitution at the 6 Position

To a solution of oxycodone-freebase (1.0 eq) in tetrahydrofuran (THF) (10 ml/mmol) was added K—O-t-butoxide (1.1 eq) or LiN(TMS)₂ (1.1 eq). After 5 minutes, Boc-Phe-OSu (1.1 eq) was added. The reaction was stirred at ambient temperatures for 18 hours, quenched with NH₄Cl, diluted with EtOAc, and solvents removed. Crude protected product was purified using chromatography. Deprotection occurred with 4N HCl in dioxane (20 ml/mmol) to obtain Phe-Oxycodone.

The following conjugates may be produced according to the above method:
Example: β-Leu-OC.

### Tripeptides

### Example 2

### General Synthesis of Mono-Substituted Tripeptide Oxycodone Conjugates Boc-Z-Y—X—O⁶-Oxycodone

To a solution of X—O⁶-Oxycodone-2HCl (1 mmol) in DMF were added NMM (10 mmol) and Boc-Z-Y—OSu (1.2 mmol). The reaction mixture was stirred at room temperature overnight. Solvent was evaporated to the residue was added saturated NaHCO₃ solution and stirred for 1 h. The precipitate was filtered, thoroughly washed with water and dried to give the title compound.

### Deprotection of Boc-Z-Y—X—O⁶-Oxycodone:

Deprotection is performed in the same manner as the general method mentioned above to give Z-Y—X—O⁶-Oxycodone.2HCl.

The following tripeptide conjugates may be produced according to the above method:
Examples: Phe-Tyr-Val-OC
   Leu-Tyr-Val-OC

### II. Disubstituted Oxycodone Conjugates

### Disubstituted Single Amino Acid Oxycodone Conjugates

### Example 3

### General Synthesis of Disubstituted Oxycodone Conjugates Containing Identical Amino Acid: [Boc-X]₂-Oxycodone

To a solution of oxycodone free base (2.04 g, 6.47 mmol) in THF (−35 ml) was added LiN(TMS)₂ (19.41 ml, 19.41 mmol) and stirred for ±30 mins. To this was added solid Boc-X—OSu (X=amino acid, 21 mmol) at one time and the reaction mixture was stirred at room temperature overnight. The solution was neutralized with 1N HCl and the THF was removed under reduced pressure. The residue was diluted with EtOAc (200 mL), satd. NaHCO₃ (150 mL) was added and stirred for 1 h. EtOAc part was washed with NaHCO₃ and brine. Dried over Na₂SO₄ and evaporated to dryness. Compound was obtained by purification over silica gel column (30% EtOAc/Hexane).

### Deprotection of [Boc-X]₂-Oxycodone:

General method of deprotection: The above compound was reacted with 4N HCl/dioxane (25 mL/gm) at room temperature for 4 h. Solvent was evaporated and dried over vacuum to give X₂-Oxycodone.3HCl.

### Example 4

### General Synthesis of Disubstituted Oxycodone Conjugates Containing Different Amino Acids Boc-X-O⁶-Oxycodone-O¹⁴-Y-Cbz

To a solution of Boc-X-Oxycodone (1 mmol) in THF (10 mL) was added LiN(TMS)₂ (1.1 mmol) at 0° C. and the solution was stirred for 30 mins then Cbz-Y—OSu (1.25 mmol) was added. The reaction mixture was stirred at room temperature overnight. The solution was cooled down to 0° C., neutralized with 1N HCl and the organic part was evaporated. To the residue were added EtOAc (50 mL) and satd. NaHCO₃ (50 ml), stirred for 1 h. The organic part was washed with water, brine, dried over Na₂SO₄ and evaporated to dryness. The residue was purified over silica gel to give the title compound.

### Deprotection of Boc-X-O⁶-Oxycodone-O¹⁴—Y-Cbz.2HCl:

Boc-X-O⁶-Oxycodone-O¹⁴—Y-Cbz was deprotected following the general method for deprotection mentioned above to give X-O⁶-Oxycodone-O¹⁴—Y-Cbz.2HCl.

### Disubstituted Tripeptide Oxycodone Conjugates

### Example 5

### Synthesis of Tripeptide-OC-Tripeptide Conjugates Containing Two Tripeptides Each Individually Having Identical Amino Acid Sequences

### Synthesis of [Boc-Val]₂-OC

To a solution of OC (2.04 g, 6.47 mmol) in tetrahydrofuran (THF) (˜35 ml) was added LiN(TMS)₂ (19.41 ml, 19.41 mmol) and stirred for ˜30 mins. To this was added solid Boc-Val-OSu (6.72 g, 21 mmol) at one time and the reaction mixture was stirred at room temperature overnight. The solution was neutralized with 1N HCl and the THF was removed under reduced pressure. The residue was diluted with ethyl acetate (EtOAc) (200 mL), satd. NaHCO₃ (150 mL) was added and stirred for 1 h. EtOAc part was washed with NaHCO₃ and brine. Dried over Na₂SO₄ and evaporated to dryness. Crude product was purified with either silica gel column. (30% EtOAc/Hexane).

Deprotection: For the deprotection of 2.5 g of [Boc-Val]₂-0° C., 75-80 mL of 4N HCl/dioxane was used. Reaction was complete within 3-4 hours. Evaporate dioxane and dry over vacuum.

Coupling: To a solution of Val₂-OC.3HCl (250 mg, 0.4 mmol) in DMF (10-12 ml) were added NMM (10-12 eqv) and Boc-X-Y—OSu (2.6 eqv). The reaction mixture was stirred at RT overnight. Solvents were evaporated under reduced pressure. To the residue was added satd. NaHCO₃ (−30 mL) and stirred for 1 h. The white/pale yellow residue was filtered, thoroughly washed with water and dried in the vacuum oven at RT.

Deprotection: Deprotection was same as above method. For 100-200 mg of tripeptide derivative 10-15 ml 4N HCl/dioxane was used.

Deprotection of Tripeptide Derivatives containing Threonine and Serine: Tripeptide derivatives were dissolved in 95% TFA (5% water) and stirred for 4 h at room temperature. Solvent was evaporated and the residue was co-evaporated with toluene twice and dried over vacuum. 4N HCl/dioxane was added and stirred overnight. Product was evaporated to dryness and dried over vacuum.

### Example 6

### Synthesis of Tripeptide-OC-Tripeptide Conjugates Containing Two Tripeptides Each Individually Having Different Amino Acid Sequences: Synthesis of [Boc-Z-Y-X]₂-Oxycodone [X, Y and Z are amino acids]

To a solution of X₂-Oxycodone 3HCl (1 mmol) in DMF (15-20 mL) were added NMM (10-12 eqv) and Boc-Z-Y—OSu (2.6 eqv). The reaction mixture was stirred at RT overnight. Solvent was evaporated under reduced pressure. To the residue was added satd. NaHCO₃ (˜30 mL) and stir for 1-2 h. The white/pale yellow residue was filtered, thoroughly washed with water and dried in the vacuum oven at room temperature.

### Deprotection of [Boc-X—Y-Z]₂-Oxycodone:

Deprotection is same as general method mentioned above. For 100-200 mg of tripeptide derivative 10-15 ml 4N HCl/dioxane is used. Deprotection is done overnight to give [X—Y-Z]₂-Oxycodone.3HCl.

### Deprotection of Tripeptide Derivatives Containing Threonine and Serine:

First the tripeptide derivatives are dissolved 95% TFA (5% water) and stirred for 4 h at room temperature. Solvent is evaporated, the residue is co-evaporated with toluene twice and dried over vacuum. 4N HCl/dioxane is added and stirred overnight. Residue was evaporated to dryness and dried over vacuum.

Synthesis of Boc-A-B-X-O⁶-Oxycodone-O¹⁴—Y-B-A-Boc (A,B,X,Y=amino acids):

To a solution of X—O⁶-Oxycodone-O¹⁴—Y.3HCl (1 mmol) and NMM (10 mmol) in DMF (10 mL) was added Boc-A-B-OSu (2.5 mmol) and the reaction mixture was stirred at room temperature overnight. Solvent was evaporated under reduced pressure and to the residue satd. NaHCO₃ (15 mL) was added and stirred for 1 h. The precipitate was filtered off and the residue was washed thoroughly with water and dried.

### Deprotection of Boc-A-B-X-O⁶-Oxycodone-O¹⁴—Y-B-A-Boc:

Deprotection is same as general method mentioned above. Deprotection is done overnight to give A-B-X-O⁶-Oxycodone-O¹⁴—Y-B-A.3HCl. Synthesis of Boc-A-B-X-O⁶-Oxycodone-O¹⁴—Y—C-D-Boc (A,B,C,D,X,Y=amino acids):

To a solution of Boc-A-B-X-O⁶-Oxycodone-O¹⁴—Y—NH₂ (1 mmol) in DMF (10 mL) were added NMM (5 mmol) and Boc-D-C-OSu (1.1 mmol) and the reaction mixture was stirred at room temperature overnight. Solvent was evaporated under reduced pressure and to the residue satd. NaHCO₃ was added and stirred for 1 h. The white precipitate was filtered, washed with water and dried.

### Deprotection of Boc-A-B-X-O⁶-Oxycodone-O¹⁴—Y-C-D-Boc:

Deprotection is same as general method mentioned above. Deprotection is done overnight to give A-B-X-O⁶-Oxycodone-O¹⁴—Y—C-D.3HCl.

### Disubstituted Tripeptide-Oxycodone-Single Amino Acid Conjugates

### Example 7

### Synthesis of Tripeptide-OC-Single Amino Acid Conjugates Containing a Tripeptide Having a Different Amino Acid Sequence:

### Synthesis of Boc-A-B-X-O⁶-Oxycodone-O¹⁴—Y-Cbz:

To a solution of X-O⁶-Oxycodone-O¹⁴-Y-Cbz.2HCl (1 mmol) and NMM (10 mmol) in DMF (10 mL) was added Boc-A-B-OSu (1.1 mmol) and the reaction mixture was stirred at room temperature overnight. Solvent was evaporated under reduced pressure and to the residue satd. NaHCO₃ (20 mL) was added and stirred vigorously for 2-3 h. The precipitate was filtered off and the residue was washed thoroughly with water and dried.

### Synthesis of Boc-A-B-X-O⁶-Oxycodone-O¹⁴—Y—NH₇

To a suspension of Boc-A-B-X-O⁶-Oxycodone-O¹⁴—Y-Cbz and Pd/C (25 Wt %) in EtOH (20 ml/gm) and cyclohexene (10 ml/gm) was heated under reflux for 30 mins. The reaction mixture was cooled down to room temperature and filtered. The filtrate was evaporated to dryness to give the title compound.

### Disubstituted Pentapeptide Oxycodone Conjugates

### Example 8

### Synthesis of Pentapeptide-OC-Pentapeptide Conjugates Containing Two Pentapeptides Each Having Different Amino Acid Sequences

### Synthesis of [Gly₂-Lys(-Gly₂)[SEQ ID NO: 36]]₂-Oxycodone

To a solution of (Gly)₂-Oxycodone (1.0 eq) in dimethylformamide (1 ml/mmol) was added 4-methylmorpholine (5.5 eq) followed by Boc-Gly₂-Lys-Gly-OSu [SEQ ID NO: 37](4.1). Reaction was stirred at ambient temperature for 24 hours. Solvents were removed and crude product was purified by reverse phase HPLC, followed by HCl deprotection gave the title compound.

### [(1)-Lys-(d)-Lys-Leu]₂-Oxycodone

To a solution of (Leu)₂-Oxycodone (1.0 eq) in dimethylformamide (1 ml/mmol) was added 4-methylmorpholine (10 eq) followed by Boc-(1)-Lys(Boc)-(d)-Lys(Boc)-OSu (3 eq). Reaction was stirred at ambient temperature for 24 hours. Solvents were removed and crude product was purified by reverse phase HPLC.

### Bioavailability Studies of Oxycodone Conjugates

The invention is illustrated by pharmacokinetic studies with oxycodone that has been covalently modified by attachment to various moieties such as an individual amino acid, specific short chained amino acid sequences such as di-, tri-, and pentapeptides, or carbohydrates such as ribose, etc. Studies include pharmacokinetic evaluations of the various drug conjugates administered by the oral, intranasal, and intravenous routes. Collectively the compounds demonstrate that active agents may be modified by covalent attachment to various moieties and retain their therapeutic value at normal doses while preventing potential overdose by oral administration and prevention of abuse through intranasal and intravenous administration.

The Examples illustrate the applicability of attaching various moieties to oxycodone to reduce the potential for overdose while maintaining therapeutic value. The invention is illustrated by pharmacokinetic studies with various peptide opioid conjugates. The Examples illustrate the compounds and compositions for reducing the potential for overdose and abuse while maintaining therapeutic value wherein the active agent oxycodone (OC) is covalently attached to a chemical moiety. The compound which is di-substituted at the 6 and 14 position of oxycodone is termed [PPL]₂—OC.

Oral, intranasal, and intravenous bioavailability studies of oxycodone and oxycodone conjugates were conducted in male Sprague-Dawley rats. Doses of oxycodone hydrochloride and oxycodone conjugates containing equivalent amounts of oxycodone were administered in deionized water. Oral administration was in 0.5 ml by gavage needle. Intranasal doses were administered by placing 20 microliters into the nasal flares of rats anesthetized with isoflurane. Intravenous administration was in 0.1 ml by tail vein injection. Plasma was collected by retroorbital sinus puncture under isoflurane anesthesia. Oxycodone and oxymorphone (major active metabolite) concentrations were determined by LC/MS/MS.

### Example 9

### Decreased Oral of Oxycodone Conjugates

Male Sprague-Dawley rats were provided water ad libitum, fasted overnight and dosed by oral gavage with oxycodone conjugates or oxycodone HCl. All doses contained equivalent amounts of oxycodone base. Plasma oxycodone concentrations were measured by ELISA (Oxymorphone, 102919, Neogen, Corporation, Lexington, Ky.) and/or LC/MS. The assay is specific for oxymorphone (the major oxycodone metabolite) and oxycodone. These examples illustrate that doses of oxycodone conjugates decrease the peak level (Cₘₐₓ) of oxycodone plus oxymorphone as compared to that produced by equimolar (oxycodone base) doses of oxycodone HCl when given by the oral route of administration.

### Example 10

### Oral Bioavailability of a Peptide-Oxycodone Conjugates at a Dose (2.5 mg/kg) Approximating a Therapeutic Human Dose

This example illustrates that when the peptide PPL is conjugated (disubstituted at the 6 and 14 positions) to the active agent oxyocodone oral bioavailability is maintained as compared to an equimolar oxyocodone dose when the dose administered is 1 mg/kg. This dose is the equivalent of a human dose of 25 to 35 mg for an individual weighing 70 kg (148 lbs) according to Chou et al.

TABLE F
Oral Pharmacokinetics of Oxycodone
vs. [PPL]₂-OC (2.5 mg/kg dose).

| | | | | | | AUC | Per- | | Per- |
|---|---|---|---|---|---|---|---|---|---|
| | Hours | | | | | (ng/ml h) | cent | Cmax | cent |
| Drug | 0.5 | 1.5 | 3 | 5 | 8 | 0-8 h | OC | ng/ml | OC |
| Oxycodone | 145 | 27 | 11 | 2 | 1 | 168 | 100 | 145 | 100 |
| Bitartrate | | | | | | | | | |
| [PPL]₂-OC | 124 | 78 | 46 | 1 | 3 | 278 | 165 | 124 | 86 |

oxycodone plus oxymorphone

### Example 11

### Bioavailability [PPL]₂-oxycodone by the Intranasal Route

This example illustrates that when [PPL]₂ is conjugated to the active agent oxycodone the bioavailability by the intranasal route is substantially decreased thereby diminishing the possibility of overdose.

### Example 12

### Bioavailability of [PPL]₂-oxycodone by the Intravenous Route

This example illustrates that when [PPL]₂ is conjugated to the active agent oxycodone the bioavailability by the intravenous route is substantially decreased thereby diminishing the possibility of overdose.

### Summary of In Vivo Testing of Abuse Resistant Oxycodone Conjugates.

In vivo testing of oxycodone conjugates demonstrates for instance decreased oral Cₘₐₓ, decreased intranasal bioavailability (AUC and Cₘₐₓ), and decreased intravenous bioavailability (AUC and Cₘₐₓ) and is described in further detail below.

### Example 13

### Decreased Intranasal Bioavailability (AUC and Cₘₐₓ) of Oxycodone Conjugates

Male Sprague-Dawley rats were provided water ad libitum and doses were administered by placing 0.02 ml of water containing oxycodone conjugates or oxycodone bitartrate into the nasal flares. All doses contained equivalent amounts of oxycodone base. Plasma oxycodone concentrations were measured by ELISA (Oxymorphone, 102919, Neogen, Corporation, Lexington, Ky.) and/or LC/MS. The assay is specific for oxymorphone (the major oxycodone metabolite) and oxycodone. These examples illustrate that oxycodone conjugates decrease the peak level (Cₘₐₓ) and total absorption (AUC) of oxycodone plus oxymorphone as compared to those produced by equimolar (oxycodone base) doses of oxycodone HCl when given by the intranasal route of administration.

### Example 14

### Decreased Intravenous Bioavailability (AUC and Cₘₐₓ) of Oxycodone Conjugates

Male Sprague-Dawley rats were provided water ad libitum and doses were administered by intravenous tail vein injection of 0.1 ml of water containing oxycodone conjugates or oxycodone HCl. All doses contained equivalent amounts of oxycodone base. Plasma oxycodone concentrations were measured by ELISA (Oxymorphone, 102919, Neogen, Corporation, Lexington, Ky.) and/or LC/MS. The assay is specific for oxymorphone (the major oxycodone metabolite) and oxycodone. This example illustrates that an oxycodone conjugate decreases the peak level (Cₘₐₓ) and total absorption (AUC) of oxycodone plus oxymorphone as compared to those produced by an equimolar (oxycodone base) dose of oxycodone HCl when given by the intravenous route of administration.

Additional bioavailability date is provided in Tables G-I for some exemplary compounds.

TABLE G
Oxycodone Compounds (Class/Oral Bioavailability)

| Formula | Class | AUC % |
|---|---|---|
| NA | NA | 100 |
| (SGV)₂-OC | Disubstituted peptide | 103 |
| (EDV)₂-OC | Disubstituted peptide | 73 |
| (VEV)₂-OC | Disubstituted peptide | 104 |
| YYV-OC | Monosubstituted peptide | 124 |
| PPV-OC | Monosubstituted peptide | 152 |
| PPI-OC | Monosubstituted peptide | 85 |
| OC-β-Alanine | Monosubstituted Single | 144 |
| | Non-natural Amino Acid | |

TABLE H
Intranasal Bioavailability of Oxycodone Compounds

| Formula | Class | AUC % |
|---|---|---|
| NA | NA | 100 |
| (SGV)₂-OC | Disubstituted peptide | 64 |
| (EDV)₂-OC | Disubstituted peptide | 20 |
| (VEV)₂-OC | Disubstituted peptide | 39 |

TABLE I
Intravenous Bioavailability of Oxycodone

| Formula | Class | AUC % |
|---|---|---|
| NA | NA | 100 |
| (SGV)₂-OC | Disubstituted peptide | 52 |

Compounds Collectively, the examples illustrate the application of the invention for reducing the overdose potential of narcotic analgesics. These examples establish that an active agent can be covalently modified by attachment of a chemical moiety in a manner that maintains therapeutic value over a normal dosing range, while substantially decreasing if not eliminating the possibility of overdose by oral, intranasal, or intravenous routes of administration with the active agent.

### Naltrexone Examples

### Example 1

### Reaction of Naltrexone-CDI Adduct with Serₙ

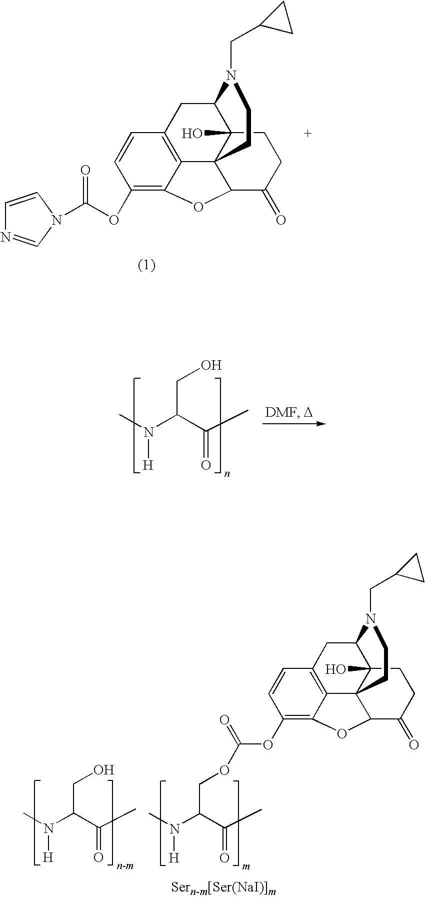

The solid from step 1 was dissolved in anhydrous N-methylpyrrolidinone (NMP), and solid Serₙ (0.51 g, 5.9 mmol) added to the solution. The reaction mixture was then heated to 60° C. under argon, and allowed to stir under argon, over night at a temperature between 50 and 60° C. The organic solution was then diluted into 100 mL of water. Precipitate formed immediately, and the solid (A) was collected by centrifuge, and the pellets then dried over night in a vacuum chamber. The water in the supernatant was removed by rotary evaporation, and the NMP solution that remained was diluted into ether (100 mL). Again, precipitate formed immediately. This solid (B) was collected by filtration and then dried over night in a vacuum chamber. Both solids were hygroscopic and appeared similar in composition by TLC (3:1 CHCl₃/CH₃OH). Therefore, solids A and B were combined and dissolved/suspended in ˜50 mL water. Ultrafiltration (1000 mw cutoff) was used to remove impurities such as unreacted naltrexone and imidazole, leaving the Serₙ and the naltrexone conjugate, Serₙ₋ₘ[Ser(Nal)]ₘ. The suspended material was washed with 5 aliquots of water (10 mL each), and then pelletted by centrifugation. The polymer conjugate was then dried over night in a vacuum chamber. This afforded 80 mg (˜5% yield) of material with an estimated loading of 1:19 naltrexone/serine (based on ¹H-NMR).

¹H NMR (360 MHz, DMSO-d₆): δ 5.03 (bs, ˜19H, α-Ser); 0.59 (bs, 2H, naltrexone-cyclopropyl) and 0.34 (bs, 2H, naltrexone-cyclopropyl).

### Example 2

### Polyserine-Naltrexone

Naltrexone, an opoid antagonist, was chosen as a model compound for testing conjugates for the hypothesis that conjugates of opoid drugs can afford extended release, while also lowering the potential for abuse. Naltrexone is chemically similar to orally delivered analgesics such as oxycodone and hydromorphone and therefore amenable to synthesizing conjugates for testing in vitro and in vivo performance.

### Synthesis

Polyserine-naltrexone (carbonate-linked) conjugates were synthesized by the following method:

1) Polymer activation. N-acetylated polyserine-methyl ester (0.69 g, 7.9 mmol) was dissolved in N-methylpyrrolidinone (15 ml) and allowed to stir under argon at ambient temperature. Carbonyldiimmidazole (CDI, 1.93 g, 11.9 mmol) was added and the reaction allowed to stir over night under argon. Then, 100 ml of acetonitrile were added and the mixture allowed to sit at 4° C. for 2 hours. The precipitate that formed was collected by centrifugation and the resulting pellet then resuspended in acetonitrile. This suspension was then centrifuged and the pellet dried over night under a vacuum.

2) Tetrabutylammonium salt of naltrexone. Naltrexone hydrochloride (1.5 g, 3.979 mmol) was dissolved in water (˜50 ml) and this solution titrated with 1N LiOH to a pH of ˜11-12. Tetrabutylammonium chloride (2.6 g, 4.0 mmol) was then added. The aqueous solution was then extracted with 3 equal volumes of chloroform (20 ml each). The organic solutions were pooled and dried with magnesium sulfate. The solvent was then removed using a rotovap, and the resulting solid dried over night under a high vacuum.

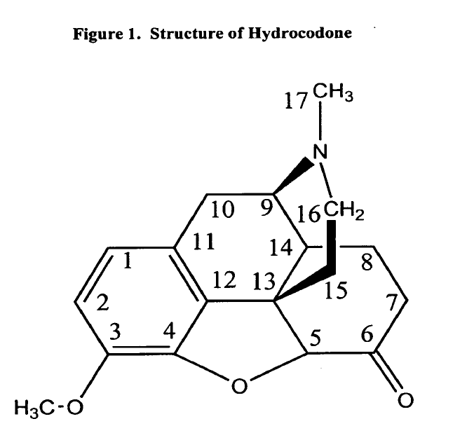

3) Conjugation reaction. The solid material from step 1 was dissolved/suspended in 15 ml of N-methylpyrrolidinone and the resulting solution placed under argon. The naltrexone salt from step 2 was then added, and the reaction then allowed to warm to ˜50-60° C. The reaction was then allowed to stir two days under these conditions, at which point water was added (˜200 ml). The aqueous solution was then concentrated by ultrafiltration (1000 mw cutoff). The concentrated solution (˜5 ml) was then diluted to a volume of 50 ml with water. The aqueous solution was then titrated to pH 3 with 1N HCl and then concentrated by ultrafiltration. This process was repeated two more times. Following the final concentration, the aqueous solution (˜5 ml) was then freed of solvent using a rotovap and high vacuum. The resulting solid was then stored over night under high vacuum. This afforded 50 mg of brown solid. A serine:naltrexone ratio of approximately 1:6 (BB272) and 1:10 (BB301) was estimated by nuclear magnetic resonance (NMR). A schematic of synthesis is shown in FIG. 1.

### Example 3

### Boc-Ser(CO-Methyl Naltrexone)-OtBu

To a solution of methyl naltrexone (1.00 g, 2.82 mmol) in THF at −78° C. was added LiN(SiMe₃)₂ (1.0M in THF, 5.92 mmol) dropwise via syringe. This solution was stirred at −78° C. for 1 hour. In a separate reaction, Boc-Ser-OtBu (0.220 g, 0.84 mmol) was dissolved in THF (5 ml) with NMM (0.10 ml, 0.92 mmol) and triphosgene (0.250 g, 0.84 mmol) added. This solution was stirred at −78° C. for 30 minutes. The first reaction was added slowly to the second at −78° C. The combined reaction was allowed to warm to ambient temperature and stirred for 18 hours. After this, water (10 ml) was added. Solvent was removed and residue was partitioned between CHCl₃/water (50 ml each) and was extracted twice with CHCl₃ (50 ml). Combined organics were washed with brine (50 ml), pH 8 water (50 ml), dried with MgSO4 and solvent removed. A preparative TLC was taken (100% CHCl3). NMR of TLC material confirmed the presence of product.

The results of the examples show that conjugation of naltrexone to a polymer of serine via a carbonate linkage can prevent spiking of the drug (decrease Cₘₐₓ) and afford sustained release (increase Tₘₐₓ while maintaining approximately equal AUC).

1. A composition for oral administration comprising (1) an analgesic or sub-analgesic amount of an opioid agonist conjugate comprising an opioid agonist covalently bound to an amino acid or peptide, and (2) an effective amount of an opioid antagonist conjugate to reduce the euphoric effect of the opioid agonist conjugate, wherein the opioid antagonist conjugate is an opioid antagonist covalently bound to an amino acid or peptide.

2. The composition of claim 1, wherein the opioid agonist is a bimodally-acting opioid agonist.

3. The composition of claim 1, wherein the composition comprises an amount of the opioid antagonist conjugate effective to enhance the analgesic potency of the covalently bound opioid agonist conjugate and to enhance the anti-analgesia, hyperalgesia, hyperexcitability, physical dependence and/or tolerance effects of the covalently bound opioid agonist conjugate.

4. (canceled)

5. The composition of claim 1, wherein the opioid antagonist is naltrexone, naloxone, or nalmefene.

6.-7. (canceled)

8. The composition of claim 1, wherein the opioid agonist is selected from the group consisting of alfentanil, allylprodine, alphaprodine, anileridine, barbiturates, benzodiazepines, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, hydrocodone, hydromorphone, hydromorphodone, hydroxypethidine, isomethadone, ketobemidone, levorphanol, levophenacylmorphan, lofentanil, meperidine, meprobamate, meptazinol, metazocine, methadone, methyldihydromorphinone, metopon, methylphenidate, morphine, myrophine, narceine, nicomorphine, norlevorphanol, normethadone, nalorphine, nalbuphene, normorphine, norpipanone, opium, oxycodone, oxymorphone, papavereturn, paregoric, pemoline, pentazocine, phenadoxone, phendimetrazine, phendimetrazone, phenomorphan, phenazocine, phenoperidine, piminodine, piritramide, propheptazine, promedol, properidine, propoxyphene, propylhexedrine, sufentanil, sufentanyl, tilidine, and tramadol.

9. The composition of claim 2, wherein the bimodally-acting opioid agonist is selected from the group consisting of morphine, codeine, pentazocine, buprenorphine, methadone, enkephalin, dynorphin and endorphin.

10.-11. (canceled)

12. The composition of claim 1, wherein the opioid agonist conjugate is an opioid agonist covalently attached to a single amino acid.

13. The composition of claim 12, wherein the single amino acid is selected from serine, glutamic acid, glycine, proline, phenylalanine, leucine, isoleucine and alpha aminoisobutyric acid.

14. The composition of claim 1, wherein the opioid agonist conjugate is an opioid agonist covalently attached to a dipeptide, tripeptide, tetrapeptide, hexapeptide, heptapeptide or pentapeptide.

15. The composition of claim 14 wherein the dipeptide is alanine-proline, pyroglutamic acid-glutamic acid or glutamic acid-glutamic acid.

16. (canceled)

17. The composition of claim 14, wherein the tripeptide is Gly-Gly-Leu, Gly-Gly-Glu, Gly-Gly-Ile, Gly-Gly-Phe, Gly-Gly-alpha aminoisobutyric acid, Gly-Leu-Ile, Gly-Phe-Ile, Gly-Leu-Leu, Gly-Phe-Leu, Leu-Pro-Glu, Leu-Pro-Leu, Leu-Pro-Phe, Pro-Pro-Glu, Pro-Pro-Leu, Pro-Pro-Ile, Pro-Pro-Phe, Glu-Glu-Glu, Leu-Leu-Glu, Leu-Leu-Leu, Glu-Pro-Val, Glu-Tyr-Val or Ile-Tyr-Val.

18.-19. (canceled)

20. The composition of claim 14, wherein the pentapeptide is Glu₅, Gly₄-Leu, Gly₄-Ile, Gly₄-Aib, Gly₄-Phe, Gly₂-Glu₃, Glu₃-Gly₂-Aib, Glu₂-Gly₂-Leu, Glu₂-Gly₂-Ile or Tyr-Tyr-Phe-Phe-Ile.

21. The composition of claim 1, wherein the opioid agonist conjugate is an opioid agonist covalently bound to a single amino acid or peptide selected from Lys, Ser, Ala, Phe, Ile, Pro-Pro-Leu, Pro-Pro-Ile, Val-Val, Lys-Lys, Gly-Gly-Ile, Phe-Phe-Ile, Phe-Phe-Leu, Thr-Thr-Val, Tyr-Tyr-Val, Tyr-Tyr-Phe, Glu-Glu-Val, Asp-Asp-Val, Lys-Lys-Val, Glu-Glu-Phe-Phe-Ile, Glu-Glu-Phe-Phe-Phe, Tyr-Tyr-Ile, Asp-Asp-Ile, Tyr-Tyr-Phe-Phe-Ile, Tyr-Tyr-Lys-Tyr-Tyr, Phe-Phe-Lys-Phe-Phe, (Lys-Lys-Gly-Gly) and [(1)-Lys-(d)-Lys-Leu]₂.

22. A method for preventing the abuse potential of an opioid composition comprising oral administrating to a patient a composition of claim 1.

23. A method for treating pain in a human subject comprising oral administrating to a patient a composition of claim 1.

24. A composition comprising (1) an analgesic or sub-analgesic amount of a G-protein coupled receptor (GPCR) agonist conjugate comprising a GPCR agonist covalently bound to an amino acid or peptide, wherein the GPCR agonist conjugate does not promote endocytosis and resensitization of the targeted GPCR and (2) an effective amount of an opioid agonist conjugate comprising a mu opioid receptor agonist covalently bound to an amino acid or peptide, wherein the mu opioid receptor agonist promotes the endocytosis of the GPCR.

25. The composition of claim 24, wherein the mu opioid receptor agonist is methadone, fentanyl, sulfentanil, remi-fentanyl, etonitazene, or etorphine.

26. A composition comprising (1) an analgesic or sub-analgesic amount of an opioid agonist conjugate comprising an opioid agonist that targets a G-protein coupled receptor (GPCR) covalently bound to a single amino acid or peptide and (2) racemic methadone, wherein the opioid agonist does not promote endocytosis and resensitization of the targeted GPCR and the methadone reduces or prevents constipation.

27. The composition of claim 26, wherein the opioid agonist is morphine.

28. The composition of claim 1, wherein the opioid agonist targets a GPCR.

29. (canceled)

30. The composition of claim 26, wherein A the racemic methadone is covalently bound to a single amino acid or peptide.

31. A composition comprising (1) an analgesic or sub-analgesic amount of an agonist conjugate comprising an agonist that targets a G-protein coupled receptor (GPCR) covalently bound to a single amino acid or peptide, wherein the agonist does not promote endocytosis and resensitization of the targeted GPCR, (2) a mu opioid receptor agonist conjugate comprising a mu opioid receptor agonist covalently bound to a single amino acid or peptide, and (3) an opioid antagonist compound comprising an opioid antagonist covalently bound to a single amino acid or peptide, wherein the mu opioid receptor agonist compound promotes the endocytosis of the GPCR, the mu opioid receptor agonist compound reduces, prevents or delays the development of tolerance to and/or physical dependence on particular drugs that target GPCRs and the opioid antagonist compound is present in an amount sufficient to prevent the euphoric effect of the covalently bound agonist and the covalently bound mu opioid receptor agonist.

32. The compound of claim 30, further comprising an amount of an opioid antagonist conjugate sufficient to prevent the euphoric effect of the covalently bound opioid agonist and said methadone, wherein the opioid antagonist conjugate is an opioid antagonist is covalently bound to a single amino acid or peptide.

33. (canceled)
